SEQUENCE LISTING (6 sequences)

SEQ ID NO: 1 - protein, 250 aa, Homo sapiens [sequence omitted]

SEQ ID NO: 2 - protein, 136 aa, Homo sapiens
MSNVPHKSSLPEGIRPGTVLRIRGLVPPNASRFHVNLLCGEEQGSDAALHFNPRLDTSEVVFNSKEQGSWGREERGPGVPFQRGQPFEVLIIASDDGFKAVVGDAQYHHFRHRLPLARVRLVEVGGDVQLDSVRIF

SEQ ID NO: 3 - protein, 139 aa, Homo sapiens
PGLVALNLGLKPGKTLTVKGTVAPKNAKRFAVNLGKGSKEENDLVLHFNPRFNEAHGDQNTVVCNSKENGDNEWGTEQREAAFPFQAGQPFEISISVEEDKFKVKVNDGHEFEFPHRLKLEAVQYLGIKGDIKLTSIKF

SEQ ID NO: 4 - protein, 317 aa, Homo sapiens
MMLSLNNLQNIIYNPVIPFVGTIPDQLDPGTLIVIRGHVPSDADRFQVDLQNGSSMKPRADVAFHFNPRFKRAGCIVCNTLINEKWGREEITYDTPFKREKSFEIVIMVLKDKFQVAVNGKHTLLYGHRIGPEKIDTLGIYGKVNIHSIGFSFSSDLQSTQASSLELTEISRENVPKSGTPQLRLPFAARLNTPMGPGRTVVVKGEVNANAKSFNVDLLAGKSKDIALHLNPRLNIKAFVRNSFLQESWGEEERNITSFPFSPGMYFEMIIYCDVREFKVAVNGVHSLEYKHRFKELSSIDTLEINGDIHLLEVRSW

SEQ ID NO: 5 - protein, 359 aa, Homo sapiens [sequence omitted]

SEQ ID NO: 6 - protein, 135 aa, Homo sapiens
MACGLVASNLNLKPGECLRVRGEVAPDAKSFVLNLGKDSNNLCLHFNPRFNAHGDANTIVCNSKDGGAWGTEQREAVFPFQPGSVAEVCITFDQANLTVKLPDGYEFKFPNRLNLEAINYMAADGDFKIKCVAFD

(19) United States
(12) Patent Application Publication
(10) Pub. No.: US 2015/0320782 A1
(43) Pub. Date: Nov. 12, 2015

(54) Methods, compositions and kits for treating, modulating, or preventing ocular angiogenesis or fibrosis in a subject using a galectin protein inhibitor

(71) Applicants: TUFTS UNIVERSITY, Boston, MA (US); GALECTO BIOTECH AB, Copenhagen (DK)

(72) Inventors: Noorjahan Panjwani, Medford, MA (US); Wei-Sheng Chen, Boston, MA (US); Hakon Leffler, Lund (SE); Ulf Nilsson, Lund (SE)

(21) Appl. No.: 14/713,079

(22) Filed: May 15, 2015

Related U.S. Application Data
(63) Continuation of application No. PCT/US2013/070306, filed on Nov. 15, 2013.
(60) Provisional application No. 61/726,998, filed on Nov. 15, 2012.

(51) Int. Cl. A61K 31/7056; A61J 1/00

(52) U.S. Cl. CPC A61K 31/7056; A61J 1/00

(57) ABSTRACT

Methods and kits using a pharmaceutical composition for use in inhibiting ocular angiogenesis or fibrosis are provided herein, such that composition includes a pharmaceutically suitable carrier or diluent and an amount of the inhibitor composition effective to inhibit the angiogenesis or the fibrosis by inhibiting expression and/or activity of a galectin protein or a portion thereof.

### RELATED APPLICATION

This application claims the benefit of international application serial number PCT/US2013/070306 filed Nov. 15, 2013 which claims the benefit of U.S. provisional application Ser. No. 61/726,998 filed Nov. 15, 2012, entitled, “Methods, compositions and kits for treating, modulating, or preventing ocular angiogenesis or fibrosis in a subject using a galectin protein inhibitor” by inventors Noorjahan Panjwani, Wei-Sheng Chen, Hakon Leffler and Ulf Nilsson, each of which is incorporated by reference herein in its entirety.

### GOVERNMENT SUPPORT

This invention was made with government support under grant EY007088 awarded by the National Institutes of Health. The government has certain rights in the invention.

### TECHNICAL FIELD

Methods and kits using a pharmaceutical composition for use in inhibiting ocular angiogenesis or ocular fibrosis in a subject are provided.

### BACKGROUND

Angiogenesis-associated disorders in the eye, such as choroidal neovascularization, result in hemorrhaging and fibrosis in the eye, and visual loss. Angiogenesis-associated disorders include age-related macular degeneration, ocular histoplasmosis syndrome, neovascular glaucoma, retrolental fibroplasia, pathologic myopia, angioid streaks, idiopathic disorders, choroiditis, choroidal rupture, overlying choroid nevi, graft rejection, herpes simplex keratitis, leishmaniasis, onchocerciasis, certain inflammatory diseases such as dry eye syndrome, and trauma to the eye (e.g., cornea).

Choroidal neovascularization is an angiogenesis-associated disorder that causes hemorrhaging, fibrosis, and visual loss (Kumar-Singh et al. International application number WO 2009/102488 published Aug. 20, 2009). The normal cornea lacks blood vessels, however in certain pathological conditions such as choroidal neovascularization, capillaries extend into the cornea from the pericorneal vascular plexus of the limbus. The cornea becomes clouded as it becomes increasingly vascularized, resulting in decreased visual acuity for patients.

Age-related macular degeneration (AMD) is the leading cause of severe vision loss in people aged 65 and above (Bressler et al. 1988 Surv. Opthalmol. 32: 375 413; Guyer et al. 1986 Arch. Opthalmol. 104: 702-705, Hyman et al. 1983 Am. J. Epidemol. 188: 816-824, Klein & Klein 1982 Arch. Opthalmol. 100: 571-573; and Leibowitz et al. 1980 Surv. Opthalmol. 24: 335-610). Although clinicopathologic descriptions have been made of AMD, little is understood about the etiology and pathogenesis of the disease.

Dry AMD is the more common form of macular degeneration, and is characterized by formation of drusen, pigmentary and atrophic changes in the macula, with slowly progressive loss of central vision. Wet or neovascular AMD is characterized by subretinal hemorrhage, fibrosis and fluid secondary to the formation of choroidal neovasculature (CNV), and more rapid and pronounced loss of vision. Neovascular AMD while less common than dry AMD accounts for 80% of the severe vision loss due to AMD. Approximately 200,000 cases of neovascular AMD are diagnosed yearly in the United States alone (See Miller et al., U.S. Pat. No. 7,125,542 issued Oct. 24, 2006 which is incorporated by reference herein in its entirety).

Currently there very few effective treatments for angiogenesis-associated disorders of the eye such as wet AMD and dry AMD. Until recently, laser photocoagulation has been the only therapy available for selected cases of neovascular AMD. Unfortunately, the majority of patients with neovascular AMD do not meet the criteria for laser photocoagulation therapy. Approximately 85% of patients with neovascular AMD have poorly defined, occult, or relatively extensive subfoveal choroidal neovascularization, none of which is amenable to laser therapy. In addition, laser photocoagulation relies on thermal damage to the CNV tissue, which damages the overlying neurosensory retina with consequent loss of visual function. Laser photocoagulation also is plagued by recurrences that occur in approximately 50% of cases.

Photodynamic therapy (PDT) has shown promising results for removing unwanted CNV and for treating neovascular AMD (Miller et al. 1999 Archives of Opthalmology 117: 1161-1173; Schmidt-Erfurth et al. 1999 Archives of Opthalmology 117: 1177-1187; TAP Study Group 1999 Archives of Opthalmology 117: 1329-1345; and Husain et al. 1999 Philadelphia: Mosby 297-307). PDT involves administering systemically a photosensitizer or PDT dye (the photosensitizer or dye accumulating in proliferating tissues such as tumors and newly formed blood vessels), and then irradiating the target tissue with low-intensity, non-thermal light at a wavelength corresponding to the absorption peak of the dye (Oleinick et al. 1998 Radiation Research 150: S146-S156). Excitation of the dye leads to formation of singlet oxygen and free radicals (better known as reactive oxygen species) which cause photochemical damage to the target tissue (Weishaupt et al. 1976 Cancer Res. 36: 2326-2329).

Studies using PDT for the treatment of CNV have demonstrated that, with the proper treatment parameters of photosensitizer dose, laser light dose, and timing of irradiation, relative selective damage to experimental CNV can be achieved, sparing retinal vessels, large choroidal vessels, and resulting in minimal changes in the neurosensory retina (Husain et al. 1996 Arch. Ophthalmology 12114: 978-985; Husain et al. 1997 Seminars in Ophthalmology 12: 14-25; Miller et al. 1995 Arch. Ophthalmology 113: 810-818; and Kramer et al. 1996 Ophthalmology 103(3): 427-438). A PDT-based procedure using a green porphyrin dye recently has been approved in a variety of countries for use in the treatment of neovascular AMD.

During clinical studies, however, investigators have reported that one to three months post-treatment recurrence of leakage occurs in at least a portion of the CNV. Increasing photosensitizer or light doses do not prevent this recurrence, and can even lead to undesired non-selective damage to retinal vessels (Miller et al. 1999 Arch. Ophthalmology 117: 1161-1173). Several multicenter Phase 3 trials are underway to study repeated PDT treatments applied every three months. Investigators hope for decreased rates of moderate vision loss using PDT treatments (TAP Study Group 1999 Arch. Ophthalmology 117: 1329-1345). However, repeated PDT treatments leads to cumulative damage to the retinal pigment epithelium (RPE) and choriocapillaris, causing progressive treatment-related vision loss.

Proteins that have been developed for the treating of AMD in subjects include antibodies specific for vascular endothelia growth factor (VEGF) such as AVASTIN™ (BEVACIZUMAB™) and LUCENTIS™ (RANIBIZUMAB™), and VEGF decoy receptors such as EYLEA™ (AFLIBERCEPT™). However, while some patients experience improvements in their conditions, healthcare providers are learning that the effects of these treatments are highly individualized based on each patient's unique biology. Thus, not every patient treated with these proteins will experience any improvement.

Several types of eye disease, notably diseases of the retina and the cornea, are accompanied by the formation of scar tissue, which contributes to visual loss, to the extent that specific inhibition of prevention of scar tissue formation is likely to result in improved visual outcome.

Scar tissue formation in the eye may arise in relation to choroidal neovascularization in age-related macular degeneration, scar tissue being most prominent after involution of the actively exudating and bleeding vascular growth phase, whence scar tissue may occupy the entire subretinal space behind the foveal photoreceptors, thus depriving the photoreceptors of the neurosensory retina of contact with a functioning retinal pigment epithelium. Currently, no method is available for the treatment of ocular fibrosis, such as retinal fibrosis, corneal fibrosis, or subretinal fibrosis and the accompanying visual loss. Prevention or inhibition of subretinal fibrosis can be achieved by photocoagulation, verteporfin-photodynamic therapy, or intravitreal pharmacologic inhibition of vascular endothelial growth factor. These treatments have not been shown, however, to be fully capable of preventing subretinal fibrosis in subretinal neovascularization. Other causes of subretinal fibrosis include degenerative myopia, choroidal rupture, outer retinal scarring and Bruch's membrane scarring following choroiditis, angioid streaks, or other causes of outer retinal injury

Scar tissue formation in the eye may occur in relation to disease that leads to the formation of preretinal and/or optic nerve head neovascularization, including for instance proliferative diabetic retinopathy, retinal vein occlusion, sickle cell disease, and uveitis. Scar tissue formation typically peaks later than new vessel formation, often concurrent with the involution of the new vessels and in the same location. Recently, clinical observations have shown that in proliferative diabetic retinopathy, intravitreal administration of a vascular endothelial growth factor inhibitor (ranibizumab or bevacizumab) can be followed by prompt regression of preretinal new vessels. Unfortunately, it has also been found that aggressive preretinal fibrosis may follow within weeks after such treatment, requiring early vitrectomy.

Scar tissue formation in the eye has also been seen following rhegmatogenous retinal detachment, a condition known as proliferative vitreoretinopathy. In this condition, fibrosis occurs without prior new vessel formation, fibrocytes believed to be formed as the result of transformation of ectopic retinal pigment epithelial cells in the vitreous and on the inner surface of the retina, or in the subretinal space. Currently, vitrectomy with preretinal membrane excision is the only generally accepted treatment for traction retinal detachment.

Scar tissue formation in the eye may occur as a result of retinopathy of prematurity, apparently as a sequel to the formation of new vessels emanating from a demarcation line between vascularized and nonvascularized tissue in the immature peripheral retina. Current methods of treatment, including cryoablation and photoablation of the avascular retina, are insufficient to fully prevent visual loss secondary to traction retinal detachment caused by contracting preretinal fibrosis membranes. Currently, vitrectomy with preretinal membrane excision is the only accepted treatment for traction retinal detachment. Invasion of the subretinal space by choroidal new vessels is a major cause of visual loss in age-related macular degeneration (AMD). Neovascular AMD benefits from treatment that specifically inhibits vascular endothelial growth factor (VEGF), but the treatment only leads to normalization of visual acuity in very few patients and approximately 30% experience considerable visual loss despite treatment

(Rosenfeld et al., NEJM 2006; 355:1419-. Brown et al. NEJM 2006; 355:1432-). Subretinal fibrosis leads to replacement of the retinal pigment epithelium by scar tissue under the neurosensory retina, with accompanying localized loss of visual function). In proliferative, i.e. primarily vasoproliferative diabetic retinopaty VEGF-inhibitor treatment using ranibizumab or bevacizumab has been reported to be capable of closing, i.e. eliminating the perfusion of pathological new vessels. Unfortunately, this potentially beneficial response is to some extent offset by an aggressive fibrosis response that follows within few weeks after the initiation of VEGF-inhibitor treatment.

Neovascularization and inflammation in the anterior segment of the eye may complicate anterior segment disease as well as retinal disease, choroidal disease, or optic nerve disease. Severe cases can be accompanied by the formation of fibrous membranes on the iris, on the ciliary body, and in the anterior chamber angle.

Because fibrosis is an integral part of the process that leads to visual loss in said diseases, because fibrosis is difficult or impossible to remove, and because damage caused by fibrosis can be difficult or impossible to repair there is a strong rationale for applying therapeutic remedies that can eliminate or reduce the fibrotic component of the said diseases.

Anterior subcapsular cataract (ASC), a type of primary cataract, is characterised by dense light scattering fibrotic regions underneath the anterior capsule. This type of cataract is associated with ocular trauma, inflammation and irritation (e.g. atopic dermatitis)

Conjunctival scarring and fibrosis appears to be a particular problem post-eye surgery, such as filtration surgery for treating glaucoma, in which fibrosis disrupts aqueous humor drainage to the trabeculectomy bleb

A range of corneal injuries—including infection (e.g. viral infection such as herpetic keratitis), surgery deep to the cornea, traumatic injury, chemical/thermal burns and inherited dystrophies—may stimulate an inflammatory healing reaction in which fibrosis results in opacification, alteration of regular curvature and decreased vision

Lens fibrosis may be observed with lens wound healing, such as postcataract surgery or after lens capsular injury. After cataract surgery, posterior capsular opacification (PCO) may commonly result in the reduction of optical transparency and deviation of the implanted lens, both of which cause decreased vision

Pterygia, which are non-malignant growths of the conjunctiva associated with chronic UV exposure, are characterised by fibrovascular tissue. Pterygia may cause local irritation, and cause vision impairment by inducing astigmatism or directly affecting the visual axis. A post-surgical risk of recurrence exists

Fibrosis is implicated in the pathophysiology of certain secondary angle closure glaucoma (e.g. the growth of a fibrovascular membrane from the iris in neovascular glaucoma results in closure of the anterior chamber angle). In addition, a body of evidence implicates trabecular meshwork ECM deposition, and resultant obstruction of aqueous draining, in the development of primary open-angle glaucoma Several growth factors have been shown experimentally to stimulate fibrosis.

Specifically, a role in promoting intraocular fibrosis and in transformation retinal pigment epithelial cells to fibrocytes has been demonstrated for transforming growth factor beta (TG F-β). There remains a need for improved compositions and methods for treating or preventing angiogenesis-associated disorders as well as fibrosis associated disorders.

### SUMMARY

An aspect of the invention provides a pharmaceutical composition for use in inhibiting ocular angiogenesis or ocular fibrosis, such that the composition includes a pharmaceutically suitable carrier or a diluent and an amount of the composition effective to inhibit or to modulate an activity of a galectin protein or portion thereof sufficient to inhibit the ocular angiogenesis or ocular fibrosis. In certain embodiments, the inhibitor binds to the portion of the galection protein including at least one amino acid of the carbohydrate recognition domain.

In various embodiments of the composition, the galectin protein is selected from the group of galectin-1, galectin-3, galectin-7, and galectin-8. The composition in various embodiments is selected from at least one of: a drug, a polymer, a protein, a peptide, a carbohydrate, a low molecular weight compound, an oligonucleotide, a polynucleotide, and a genetic material such as DNA or RNA. For example the genetic material is an siRNA that negatively modulates expression of the galectin. The drug in various embodiments is a therapeutically, prophylactically and/or pharmacologically or physiologically beneficial active agent, substance, or mixture thereof, which is delivered to cells or to a subject to produce a desired beneficial effect. The drug comprises for example a protein, a peptide, or nucleic acids.

The composition in various embodiments is effective to treat or prevent a disease or a condition associated with the ocular angiogenesis. As used herein, the phrase ocular “disease or the condition” means any disorder characterized by at least one of: excessive neovascularization, trauma or injury to the eye, a surgery associated with scarring for example a glaucoma filtration surgery; neovascular glaucoma; a corneal injury; post-conjunctivitis scarring; pterygium, age related-macular degeneration (AMD) for example wet AMD or dry AMD; conversion from dry to wet AMD; proliferative diabetic retinopathy; diabetic macular edema; and corneal neovascularization (trachoma). Alternatively, the phrase “disorder or condition” is any pathology caused by or resulting from ocular angiogenesis. In various embodiments, the disorder or condition is related to ocular neovascularization, retinal edema, diabetic retinopathy, sequela associated with retinal ischemia, posterior segment neovascularization, and neovascular glaucoma.

The composition in various embodiments is effective to treat or prevent a disease or a condition associated with the ocular fibrosis. As used herein, the phrase ocular “disease or the condition” means any disorder characterized by at least one of: excessive fibrosis or collagen deposition in one or more anatomical locations in the eye, fibrosis in particular due to trauma or other injury to the eye, a surgery inducing scarring for example a glaucoma filtration surgery; neovascular glaucoma; a corneal injury; post-conjunctivitis scarring; pterygium, age related-macular degeneration (AMD) for example wet AMD or dry AMD; conversion from dry to wet AMD; proliferative diabetic retinopathy; diabetic macular edema; pathological myopia, retinopaty of prematurity, retinal pigment epithelial detachment, Stevens-Johnson Syndrome, uveitis, anterior subcapsular cataract, dry eye disease and corneal neovascularization (trachoma). Alternatively, the phrase “disorder or condition” is any pathology caused by or resulting from ocular fibrosis. In various embodiments, the disorder or condition is related to ocular fibrosis, retinal scarring, diabetic retinopathy, sequela associated with retinal ischemia, and glaucoma.

The composition in various embodiments is a beta-galactoside. In various embodiments the composition is derivatized or functionalized. In various embodiments the composition is non-metabolizable, or in alternative embodiments is metabolizable.

The composition in a related embodiment further includes at least one agent selected from the group consisting of: anti-angiogenic, anti-tumor, antiviral, antibacterial, anti-mycobacterial, anti-fungal, anti-proliferative, anti-inflammatory, and anti-apoptotic. For example, the agent is an anti-bacterial molecule that prevents an infection in the eye.

The composition for use in inhibiting ocular angiogenesis or ocular fibrosis includes in various embodiments the following general formula:

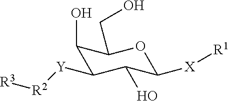

such that the configuration of the pyranose ring is D-galacto; X is selected from the group consisting of O, S, NH, CH₂, and NR⁴, or is a bond; Y is selected from the group consisting of NH, CH₂, and NR⁴, or is a bond; R¹ is selected from the group consisting of: a saccharide; hydrogen, an alkyl group, an alkenyl group, an aryl group, a heteroaryl group, and a heterocycle; R² is selected from the group consisting of CO, SO₂, SO, PO, and PO₂; R³ is selected from the group consisting of: an alkyl group of at least 4 carbon atoms, an alkenyl group of at least 4 carbon atoms, an alkyl or alkenyl group of at least 4 carbon atoms substituted with a carboxy group, an alkyl group of at least 4 carbon atoms substituted with both a carboxy group and an amino group, and an alkyl group of at least 4 carbon atoms substituted with a halogen; a phenyl group, a phenyl group substituted with a carboxy group, a phenyl group substituted with at least one halogen, a phenyl group substituted with an alkoxy group, a phenyl group substituted with at least one halogen and at least one carboxy group, a phenyl group substituted with at least one halogen and at least one alkoxy group, a phenyl group substituted with a nitro group, a phenyl group substituted with a sulfo group, a phenyl group substituted with an amine group, a phenyl group substituted with a hydroxy group, a phenyl group substituted with a carbonyl group and a phenyl group substituted with a substituted carbonyl group; and a phenyl amino group; or R⁴ is selected from the group consisting of hydrogen, an alkyl group, an alkenyl group, an aryl group, a heteroaryl group, and a heterocycle.

In related embodiments of the composition, the saccharide (R¹) is selected from the group consisting of glucose, mannose, galactose, N-acetylglucosamine, N-acetylgalactosamine, fucose, fructose, xylose, sialic acid, glucuronic acid, iduronic acid, a disaccharide or, an oligosaccharide comprising at least two of the above saccharides, and derivatives thereof.

In various embodiment of the composition, Y is NH; X is O; or the halogen is selected from the group consisting of F, Cl, Br and I.

The composition in various embodiments is selected from: methyl 2-acetamido-2-deoxy-4-O-(3-[3-carboxypropanamido]-3-deoxy-β-D-galactopyranosyl)-β-D-glucopyranoside; methyl 2-acetamido-2-deoxy-4-O-(3-[{Z}-3-carboxypropenamido]-3-deoxy-β-D-galactopyranosyl)-β-D-glucopyranoside; methyl 2-acetamido-2-deoxy-4-O-(3-benzamido-3-deoxy-β-D-galactopyranosyl)-β-D-glucopyranoside; methyl 2-acetamido-2-deoxy-4-O-(3-[2-carboxy-benzamido]-3-deoxy-β-D-galactopyranosyl)-β-D-glucopyranoside; methyl 2-acetamido-2-deoxy-4-O-(3-[4-methoxy-2,3,5,6-tetrafluorbenz-amido]-3-deoxy-β-D-galactopyranosyl)-β-D-glucopyranoside; methyl 2-acetamido-2-deoxy-4-O-(3-[2-carboxy-3,4,5,6-tetrafluorbenzamido]-3-deoxy-β-D-galactopyranosyl)-β-D-glucopyranoside; methyl 2-acetamido-2-deoxy-4-O-(3-methanesulfonamido-3-deoxy-β-D-galactopyranosyl)-β-D-glucopyranoside; methyl 2-acetamido-2-deoxy-4-O-(3-[-4-nitrobenzenesulfonamido]-3-deoxy-β-D-galactopyranosyl)-β-D-glucopyranoside, methyl 2-acetamido-2-deoxy-4-O-(3-phenylaminocarbonylam-ino-3-deoxy-β-D-galactopyranosyl)-β-D-glucopyranoside; methyl 2-acetamido-2-deoxy-4-O-(3-aminoacetamido-3-deoxy-β-D-galactopyranosyl)-β-D-glucopyranoside; methyl 2-acetamido-2-deoxy-4-O-(-3-[{2S}-2-amino-3-carboxy-propanamido]-3-deoxy-β-D-galactopyranosyl)-β-D-glucopyranoside.

The composition in various embodiments includes the general formula:

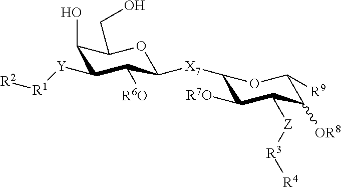

such that the configuration of one of the pyranose rings is β-D-galacto; X is selected from the group consisting of O, S, SO, SO₂, NH, CH₂, and NR⁵; Y is selected from the group consisting of O, S, NH, CH₂, and NR⁵, or is a bond; Z is selected from the group consisting of O, S, NH, CH₂, and NR⁵, or is a bond; R¹ and R³ are independently selected from the group consisting of CO, SO₂, SO, PO₂, PO, and CH₂ or is a bond; R² and R⁴ are independently selected from the group consisting of: an alkyl group of at least 4 carbons, an alkenyl group of at least 4 carbons, an alkyl group of at least 4 carbons substituted with a carboxy group, an alkenyl group of at least 4 carbons substituted with a carboxy group, an alkyl group of at least 4 carbons substituted with an amino group, an alkenyl group of at least 4 carbons substituted with an amino group, an alkyl group of at least 4 carbons substituted with both an amino and a carboxy group, an alkenyl group of at least 4 carbons substituted with both an amino and a carboxy group, and an alkyl group substituted with one or more halogens; a phenyl group substituted with at least one carboxy group, a phenyl group substituted with at least one halogen, a phenyl group substituted with at least one alkoxy group, a phenyl group substituted with at least one nitro group, a phenyl group substituted with at least one sulfo group, a phenyl group substituted with at least one amino group, a phenyl group substituted with at least one alkylamino group, a phenyl group substituted with at least one arylamino group, a phenyl group substituted with at least one dialkylamnino group, a phenyl group substituted with at least one hydroxy group, a phenyl group substituted with at least one carbonyl group and a phenyl group substituted with at least one substituted carbonyl group; or a naphthyl group, a naphthyl group substituted with at least one carboxy group, a naphthyl group substituted with at least one halogen, a naphthyl group substituted with at least one alkoxy group, a naphthyl group substituted with at least one nitro group, a naphthyl group substituted with at least one sulfo group, a naphthyl group substituted with at least one amino group, a naphthyl group substituted with at least one alkylamino group, a naphthyl group substituted with at least one arylamino group, a naphthyl group substituted with at least one dialkylamnino group, a naphthyl group substituted with at least one hydroxy group, a naphthyl group substituted with at least one carbonyl group and a naphthyl group substituted with at least one substituted carbonyl group; a heteroaryl group, a heteroaryl group substituted with at least one carboxy group, a heteroaryl group substituted with at least one halogen, a heteroaryl group substituted with at least one alkoxy group, a heteroaryl group substituted with at least one nitro group, a heteroaryl group substituted with at least one sulfo group, a heteroaryl group substituted with at least one amino group, a heteroaryl group substituted with at least one alkylamino group, a heteroaryl group substituted with at least one dialkylamino group, a heteroaryl group substituted with at least one arylamino group, a heteroaryl group substituted with at least one hydroxy group, a heteroaryl group substituted with at least one carbonyl group and a heteroaryl group substituted with at least one substituted carbonyl group; R⁶ and R⁸ are independently selected from the group consisting of a hydrogen, an acyl group, an alkyl group, a benzyl group, and a saccharide; R⁷ is selected from the group consisting of a hydrogen, an acyl group, an alkyl group, and a benzyl group; and/or R⁹ is selected from the group consisting of a hydrogen, a methyl group. hydroxymethyl group, an acyloxymethyl group, an alkoxymethyl group, and a benzyloxymethyl group.

In various embodiments of the composition, Y is NH. In various embodiments of the composition, Z is NH. In various embodiments of the composition, X is S. In various embodiments of the composition, R¹ is CO. In various embodiments of the composition, R³ is CO. In various embodiments of the composition, R² or R⁴ is an aromatic for example an aromatic ring; either of R⁶, R⁷, and R⁸ is hydrogen; or R⁹ is a hydroxymethyl group.

The composition in various embodiments is bis-(3-deoxy-3-benzamido-β-D-galactopyranosyl)sulfane (17), bis-(3-deoxy-3-(3-methoxybenzamido)-β-D-galactopyranosyl)sulfane; bis-(3-deoxy-(3,5-dimethoxybenzamido)-β-D-galactopyranosyl)sulfane; bis-(3-deoxy-3-(4-nitrobenzamido)-β-D-galactopyranosyl)sulfane; bis(3-deoxy-3-(2-naphthamido)-β-D-galactopyranosyl)sulfane; bis-(3-deoxy-3-(4-methoxybenzamido)-β-D-galactopyranosyl)sulfane; bis-(3-deoxy-3-(3-nitrobenzamido)-β-D-galactopyranosyl)sulfane; bis-(3-deoxy-3-[4-(dimethylamino)-benzamido]-β-D-galactopyranosyl)sulfane; bis-(3-deoxy-3-(4-methylbenzamido)-β-D-galactopyranosyl)sulfane; bis-(3-deoxy-3-(4-chlorobenzamido)-β-D-galactopyranosyl)sulfane; bis-(3-deoxy-3-(4-tert-butylbenzamido)-β-D-galactopyranosyl)sulfane; bis-(3-deoxy-3-(4-acetylbenzamido)-β-D-galactopyranosyl)sulfane; bis-(3-deoxy-3-[2-(3-carboxy)-naphthamido]-β-D-galactopyranosyl)sulfane; bis-[3-deoxy-3-(3,4-methylenedioxyl)benzamido]-β-D-galactopyranosyl)sulfane, bis-(3-deoxy-3-(4-methoxycarbonylbenzamido)-β-D-galactopyranosyl)sulfane; bis-(3-deoxy-3-(3-carboxybenzamido)-β-D-galactopyranosyl)sulfane; bis-(3-deoxy-3-(3-benzyloxy-5-hydroxy-benzamido)-β-D-galactopyranosyl)sulfane; bis-(3-deoxy-3-(3,5-dibenzyloxybenzamido)-β-D-galactopyranosyl)sulfane; bis-(3-deoxy-3-(3-benzyloxy-5-methoxy-benzamido)-β-D-galactopyranosyl)sulfane; bis-(3-deoxy-3-(3-benzyloxy-5-nonyloxy-benzamido)-β-D-galactopyranosyl)sulfane; bis-(3-deoxy-3-(3-hydroxy-5-methoxy-benzamido)-β-D-galactopyranosyl)-sulfane; bis-(3-deoxy-3-(3-hydroxy-5-nonyloxy-benzamido)-β-D-galactopyranosyl)sulfane, bis-(3-deoxy-3-[3-benzyloxy-5-(4-fluoro-benzyloxy)-benzamido]-β-D-galactopyranosyl)sulfane; bis-(3-deoxy-3-[3-methoxy-5-(4-methyl-benzyloxy)-benzamido]-β-D-galactopyranosyl)sulfane; or bis-(3-deoxy-3-(3-allyloxy-5-benzyloxy-benzamido)-β-D-galactopyranosyl)sulfane.

The composition includes in various embodiments a galactoside for example 3-triaxolyl-galactoside. In various embodiments, the composition includes a general formula shown below:

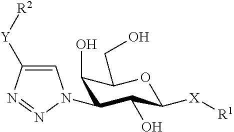

such that the configuration of the pyranose ring is D-galacto; X is selected from the group consisting of O, S, NH, CH₂, and NR⁴, or is a bond; Y is selected from the group consisting of CH₂, CO, SO₂, SO, PO₂ and PO, phenyl, or is a bond; R¹ is selected from the group consisting of: a saccharide; a substituted saccharide; D-galactose; substituted D-galactose; C3-[1,2,3]-triazol-1-yl-substituted D-galactose; hydrogen, an alkyl group, an alkenyl group, an aryl group, a heteroaryl group, and a heterocycle and derivatives thereof; and an amino group, a substituted amino group, an imino group, or a substituted imino group; and/or R² is selected from the group consisting of; hydrogen, an amino group, a substituted amino group, an alkyl group, a substituted alkyl group, an alkenyl group, a substituted alkenyl group, an alkynyl group, a substituted alkynyl group, an alkoxy group, a substituted alkoxy group, an alkylamino group, a substituted alkylamino group, an arylamino group, a substituted arylamino group, an aryloxy group, a substituted aryloxy group, an aryl group, a substituted aryl group, a heteroaryl group, a substituted heteroaryl group, and a heterocycle, a substituted heterocycle.

The saccharide in related embodiments of the composition is selected from the group consisting of glucose, mannose, galactose, N-acetylglucosamine, N-acetylgalactosamine, fucose, fructose, xylose, sialic acid, glucuronic acid, iduronic acid, galacturonic acid, a disaccharide or an oligosaccharide comprising at least two of the above saccharides, and derivatives thereof.

In various embodiments of the composition, Y is CO, SO₂, or a bond. In various embodiments of the composition, R² is an amine or an aryl group. In various embodiments of the composition, R² is a substituted phenyl group wherein said substituent is one or more selected from the group consisting of halogen, alkoxy, alkyl, nitro, sulfo, amino, hydroxy or carbonyl group. In various embodiments of the composition, R¹ is galactose, glucose or N-acetylglucosamine. In various embodiments of the composition, R¹ is a substituted galactose. In various embodiments of the composition, R¹ is either a substituted galactose, a substituted glucose, or a substituted N-acetylglucosamine. In various embodiments of the composition, R¹ is a C3-[1,2,3]-triazol-1-yl-substituted galactose. In various embodiments of the composition, X is O or S.

The composition in various embodiments includes at least one of the group selected from: methyl 3-deoxy-3-(1H-[1,2,3]-triazol-1-yl)-1-thio-β-D-galactopyranoside; methyl 3-deoxy-3-(4-propyl-1H-[1,2,3]-triazol-1-yl)-1-thio-β-D-galactopyranoside; methyl 3-(4-methoxycarbonyl-1H-[1,2,3]-triazol-1-yl)-3-deoxy-1-thio-β-D-galactopyranoside; methyl 3-deoxy-3-(4-(1-hydroxy-1-cyclohexyl)-1H-[1,2,3]-triazol-1-yl)-1-thio-β-D-galactopyranoside; methyl 3-deoxy-3-(4-phenyl-1H-[1,2,3]-triazol-1-yl)-1-thio-β-D-galactopyranoside; methyl 3-deoxy-3-(4-p-tolylsulfonyl-1H-[1,2,3]-triazol-1-yl)-1-thio-β-galactopyranoside; methyl 3-(4-methylaminocarbonyl-1H-[1,2,3]-triazol-1-yl)-3-deoxy-1-thio-β-D-galactopyranoside; methyl 3-(4-butylaminocarbonyl-1H-[1,2,3]-triazol-1-yl)-3-deoxy-1-thio-β-D-galactopyranoside; methyl 3-(4-benzylaminocarbonyl-1H-[1,2,3]-triazol-1-yl)-3-deoxy-1-thio-β-D-galactopyranoside; methyl 3-{4-(3-hydroxyprop-1-ylaminocarbonyl)-1H-[1,2,3]-triazol-1-yl}-3-deoxy-1-thio-β-D-galactopyranoside; methyl 3-{4-[2-(N-morpholino)-ethylaminocarbonyl]-1H-[1,2,3]-triazol-1-yl}-3-deoxy-1-thio-β-D-galactopyranoside; methyl 3-(4-methylaminocarbonyl-1H-[1,2,3]-triazol-1-yl)-3-deoxy-β-D-galactopyranosyl-(1→4)-2-acetamido-2-deoxy-β-D-glucopyranoside, bis-(3-deoxy-3-(4-(methylaminocarbonyl)-1H-[1,2,3]-triazol-1-yl)-β-D-galactopyranosyl)sulfane, methyl 3-deoxy-3-{4-(2-fluorophenyl)-1H-[1,2,3]-triazol-1-yl}-1-thio-β-D-galactopyranoside; methyl 3-deoxy-3-{4-(2-methoxyphenyl)-1H-[1,2,3]-triazol-1-yl}-1-thio-β-D-galactopyranoside; methyl 3-deoxy-3-{4-(3-methoxyphenyl)-1H-[1,2,3]-triazol-1-yl}-1-thio-β-D-galactopyranoside; methyl 3 deoxy-3-{4-(4-methoxyphenyl)-1H-[1,2,3]-triazol-1-yl}-1-thio-β-D-galactopyranoside; methyl 3-deoxy-3-{4-(3,5-dimethoxyphenyl)-1H-[1,2,3]-triazol-1-yl}-1-thio-β-D-galactopyranoside; methyl 3-deoxy-3-{4-(1-naphthyl)-1H-[1,2,3]-triazol-1-yl}-1-thio-β-D-galactopyranoside; methyl 3-deoxy-3-{4-(2-naphthyl)-1H-[1,2,3]-triazol-1-yl}-1-thio-β-D-galactopyranoside; methyl 3-deoxy-3-{4-(2-pyridyl)-1H-[1,2,3]-triazol-1-yl}-1-thio-β-D-galactopyranoside; methyl 3-deoxy-3-{4-(3-pyridyl)-1H-[1,2,3]-triazol-1-yl}-1-thio-β-D-galactopyranoside; methyl 3-deoxy-3-{4-(4-pyridyl)-1H-[1,2,3]-triazol-1-yl}-1-thio-β-D-galactopyranoside; O-{3-deoxy-3-{4-phenyl-[1H-[1,2,3]-triazol-1-yl}-β-D-galactopyranosy-l}-3-indol-carbaldoxim; O-{3-deoxy-3-[4-(methylaminocarbonyl)-1H-[1,2,3]-triazol-1-yl]-β-D-galactopyranosyl}-3-indol-carbaldoxim; O-{3-deoxy-3-[4-phenyl-[1H-[1,2,3]-triazol-1-yl]-β-D-galactopyranosy-l}-(2-hydroxy-5-nitro-phenyl)-carbaldoxim; O-{3-deoxy-3-[4-(methylaminocarbonyl)-1H-[1,2,3]-triazol-1-yl]-β-D-galactopyranosyl}-(2-hydroxy-5-nitro-phenyl)-carbaldoxim; O-{3-deoxy-3-[4-phenyl-[1H-[1,2,3]-triazol-1-yl]-β-D-galactopyranosy-1}-(2,5-dihydroxyphenyl)-carbaldoxim; O-{3-deoxy-3-[4-(methylaminocarbonyl)-1H-[1,2,3]-triazol-1-yl]-β-D-galactopyranosyl}-(2,5-dihydroxyphenyl)-carbaldoxim; O-{3-deoxy-3-[4-phenyl-[1H-[1,2,3]-triazol-1-yl]-β-D-galactopyranosyl}-1-naphthyl-carbaldoxim; or O-{3-deoxy-3-[4-(methylaminocarbonyl)-1H-[1,2,3]-triazol-1-yl]-β-D-galactopyranosyl}-1-naphthyl-carbaldoxim.

The composition in various embodiments includes a digalactoside or a thiodigalactoside, for example the composition includes a general formula shown below:

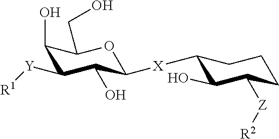

such that the configuration of the pyranose ring is D-galacto; X is selected from the group consisting of O, S, and SO; Y and Z are independently selected from: CONH or a 1H-1,2,3-triazole ring; R¹ and R² are independently selected from the group consisting of: an alkyl group of at least 4 carbons, an alkenyl group of at least 4 carbons, an alkynyl group of at least 4 carbons; a carbamoyl group, a carbamoyl group substituted with an alkyl group, a carbamoyl group substituted with an alkenyl group, a carbamoyl group substituted with an alkynyl group, a carbamoyl group substituted with an aryl group, a carbamoyl group substituted with an substituted alkyl group, and a carbamoyl group substituted with an substituted aryl group; a phenyl group substituted with at least one carboxy group, a phenyl group substituted with at least one halogen, a phenyl group substituted with at least one alkyl group, a phenyl group substituted with at least one alkoxy group, a phenyl group substituted with at least one trifluoromethyl group; a phenyl group substituted with at least one trifluoromethoxy group, a phenyl group substituted with at least one sulfo group, a phenyl group substituted with at least one hydroxy group, a phenyl group substituted with at least one carbonyl group, and a phenyl group substituted with at least one substituted carbonyl group; a naphthyl group, a naphthyl group substituted with at least one carboxy group, a naphthyl group substituted with at least one halogen, a naphthyl group substituted with at least one alkyl group, a naphthyl group substituted with at least one alkoxy group, a naphthyl group substituted with at least one sulfo group, a naphthyl group substituted with at least one hydroxy group, a naphthyl group substituted with at least one carbonyl group, and a naphthyl group substituted with at least one substituted carbonyl group; a heteroaryl group, a heteroaryl group substituted with at least one carboxy group, a heteroaryl group substituted with at least one halogen, a heteroaryl group substituted with at least one alkoxy group, a heteroaryl group substituted with at least one sulfo group, a heteroaryl group substituted with at least one arylamino group, a heteroaryl group substituted with at least one hydroxy group, a heteroaryl group substituted with at least one halogen, a heteroaryl group substituted with at least one carbonyl group, and a heteroaryl group substituted with at least one substituted carbonyl group; and a thienyl group, a thienyl group substituted with at least one carboxy group, a thienyl group substituted with at least one halogen, a thienyl thienyl group substituted with at least one alkoxy group, a thienyl group substituted with at least one sulfo group, a thienyl group substituted with at least one arylamino group, a thienyl group substituted with at least one hydroxy group, a thienyl group substituted with at least one halogen, a thienyl group substituted with at least one carbonyl group, and a thienyl group substituted with at least one substituted carbonyl group.

In various embodiments of the composition, Y is CONH for example the CONH group is linked via the N atom to the pyranose ring. In various embodiments of the composition, Z is CONH. In various embodiments of the composition, the CONH group is linked via the N atom to the cyclohexane. In various embodiments of the composition, Y is a 1H-1,2,3-triazole ring for example in a related embodiment of the composition the 1H-1,2,3-triazole ring is linked via the N1 atom to the pyranose ring. In various embodiments of the composition, R¹ is linked to the C4 atom of the 1H-1,2,3-triazole ring.

In various embodiments of the composition, Z is a 1H-1,2,3-triazole ring. For example, the 1H-1,2,3-triazole ring is linked via the N1 atom to the cyclohexane. In various embodiments of the composition, R² is linked to the C4 atom of the 1H-1,2,3-triazole ring.

In various embodiments of the composition, R¹ and R² are independently selected from the group consisting of a carbamoyl group, an alkylated carbamoyl group, an alkenylated carbamoyl group, an arylated carbamoyl group, a phenyl group, a substituted phenyl group, a halogenated phenyl group, a fluorinated phenyl group, a chlorinated phenyl group, a brominated phenyl group, an alkylated phenyl group, an alkenylated phenyl group, a trifluoromethylated phenyl group, a methoxylated phenyl group, a trifluoromethoxylated phenyl group, a naphthyl group, a substituted naphthyl group, a heteroaryl group, a substituted heteroaryl group, a thienyl group, and a substituted thienyl group.

In various embodiments of the composition, R¹ is an alkylated carbamoyl group, a fluorinated phenyl group, or a thienyl group. In various embodiments of the composition, R² is an alkylated carbamoyl group, a fluorinated phenyl group, or a thienyl group. In various embodiments of the composition, X is O or S.

The composition in various embodiments is selected from the group consisting of: ((1R,2R,3S)-2-hydroxy-3-(4-(N-(1-propyl)-carbamoyl)-1H-1,2,3-triazol-1-yl)cyclohexyl) 3-deoxy-(3-(4-(N-(1-propyl)-carbamoyl)-1H-1,2,3-triazol-1-yl))-β-D-galactopyranoside; ((1R,2R,3S)-2-hydroxy-3-(4-(2-fluorophenyl)-1H-1,2,3-triazol-1-yl)-cyclohexyl) 3-deoxy-3-(4-(2-fluorophenyl)-1H-1,2,3-triazol-1-yl)-1-thio-β-D-galactopyranoside; ((1R,2R,3S)-2-hydroxy-3-(4-(3-fluorophenyl)-1H-1,2,3-triazol-1-yl)-cyclohexyl) 3-deoxy-3-(4-(3-fluorophenyl)-1H-1,2,3-triazol-1-yl)-1-thio-β-D-galactopyranoside; ((1R,2R,3S)-2-hydroxy-3-(4-(4-fluorophenyl)-1H-1,2,3-triazol-1-yl)-cyclohexyl) 3-deoxy-3-(4-(4-fluorophenyl)-1H-1,2,3-triazol-1-yl)-1-thio-β-D-galactopyranoside; (1R,2R,3S)-2-hydroxy-3-(4-(3-thienyl)-1H-1,2,3-triazol-1-yl)-cyclohexyl) 3-deoxy-3-(4-(3-thienyl)-1H-1,2,3-triazol-1-yl)-1-thio-β-D-galactopyranoside; (1R,2R,3S)-2-hydroxy-3-(4-(N-(1-propyl)-carbamoyl)-1H-1,2,3-triazol-1-yl)-cyclohexyl) 3-deoxy-3-(4-(N-(1-propyl)-carbamoyl)-1H-1,2,3-triazol-1-yl)-1-thio-β-D-galactopyranoside, and (1R,2R,3S)-2-hydroxy-3-(4-chlorobenzamido)-cyclohexyl) 3-deoxy-3-(4-chlorobenzamido)-1-thio-β-D-galactopyranoside. In various embodiments of the composition, the inhibitor comprises TD139, Compound 32, or a portion or homolog thereof.

The composition in various embodiments includes a digalactoside, for example the composition includes a general formula (13)

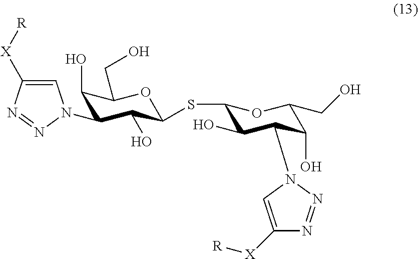

wherein the configuration of at least one of the pyranose rings is D-galacto; X is a bond; R is a phenyl group, which is substituted in any position with one or more substituents selected from the group consisting of methyl, ethyl, isopropyl, tert-butyl, fluoro, chloro, bromo, and trifluoromethyl or R is a thienyl group.

In various embodiments of the composition R is a phenyl group which is substituted in any position with one or more substituents selected from the group consisting of fluoro, chloro, and bromo. For instance R is a phenyl group which is substituted in any position with one or more substituents selected from fluoro. Typically, the configuration of both pyranose rings is D-galacto.

An aspect of the invention provides a method for treating or preventing an ocular angiogenesis or ocular fibrosis in a subject, the method comprising administering a therapeutically effective amount of at least one composition to the subject, such that the composition includes an inhibitor of a galectin protein or a portion thereof. The subject is for example a mammal such as a human, a dog, a cat, a horse, a pig, a rodent, and a cow.

The ocular angiogenesis or the ocular fibrosis in various embodiments of the method is associated with a disease or a condition which is conventionally treated by a surgery associated with scarring for example a glaucoma filtration surgery; neovascular glaucoma; a corneal injury; post-conjunctivitis scarring; pterygium, AMD for example wet AMD or dry AMD; conversion from dry to wet AMD; proliferative diabetic retinopathy; diabetic macular adema; or corneal neovascularization (trachoma).

The galectin protein in various embodiments of the method is selected from the group of: galectin-1 protein, galectin-3 protein, galectin-7 protein, and galectin-8 protein, and such that the composition is selected from at least one of: a drug, a polymer, a protein, a peptide, a carbohydrate, a low molecular weight compound, an oligonucleotide, a polynucleotide, and a genetic material such as DNA or RNA.

The method in various embodiments further includes observing reduction in a parameter or an indicator of the disease or the condition, for example the parameter or the indicator is a marker. For example, the marker is a molecule (e.g., a growth factor) that indicates the presence or absence of the ocular angiogenesis or ocular fibrosis.

The method in various embodiments further includes, prior to administering, engineering the composition, such that the composition binds to the galectin protein and modulates a VEGF/VEGF receptor-2 pathway, or the composition modulates expression of the galectin protein. The method in various embodiments further includes, prior to administering, engineering the composition, such that the composition binds to the galectin protein and modulates a fibroblast growth factor pathway, or the composition modulates expression of the galectin protein.

An aspect of the invention provides a method for treating or preventing an ocular angiogenesis or ocular fibrosis in a subject, the method including administering a therapeutically effective amount of at least one composition to the subject, such that the composition includes an inhibitor of a galectin protein or a portion thereof, such that the administering includes contacting the subject with any of the pharmaceutical compositions described herein. In various embodiments of the method, the subject is a mammal.

The method in various embodiments further includes observing or detecting a reduction in fibrosis. In various embodiments of the method, administering includes contacting the subject or tissue of the subject with a composition containing dose of at least: about 0.01 nanograms (ng) to about 1 ng, about 1 ng to about 10 ng, about 10 ng to about 20 ng, about 20 ng to about 30 ng, about 30 ng to about 40 ng, about 40 ng to about 50 ng, about 50 ng to about 100 ng, 100 ng to about 200 ng, 200 ng to about 300 ng, about 300 ng to about 400 ng, about 400 ng to about 600 ng, about 600 ng to about 800 ng, about 1 microgram (μg) to about 5 μg, about 5 μg to about 20 μg, about 20 μg to about 40 μg, about 40 μg to about 60 μg, about 60 μg to about 80 μg, about 80 μg to about 100 μg, about 100 μg to about 200 μg, about 200 μg to about 300 μg, and about 300 μg to about 400 μg. For example the dose is about 0.01 ng to about 10 ng, or about 10 ng to about 100 ng, our about 100 ng to about 500 ng, our about 500 ng to about 2 μg, or about 2 μg to about 250 μg, or about 250 μg to about 750 μg.

In various embodiments, prior to administering to the subject, the method further includes engineering a polynucleotide sequences which encodes the inhibitor, for example the inhibitor is a binding protein that mimics the carbohydrate recognition domain of galectin-1, galectin-3, galectin-7 or galectin-8. For example, the inhibitor is a fusion protein.

The method in various embodiments further includes, prior to administering, formulating the composition for an ocular delivery selected from the group of: an injection, an eye drop, a patch, an ointment, a gel, or a spray. For example, the inhibitor is formulated in a composition that is pH neutral, buffered, and isotonic.

Administering the composition to the subject in various embodiments of the method includes injecting the composition by a route such as intra-ocularly, intravitreally, corneally, conjunctively, sub-conjunctively, intravenously, or tenonly. In various embodiments, the ocular angiogenesis or ocular fibrosis is related to ocular neovascularization, retinal edema, diabetic retinopathy, sequela associated with retinal ischemia, posterior segment neovascularization, and neovascular glaucoma. In various embodiments of the method, the inhibitor comprises TD139, Compound 32, or a portion or homolog thereof.

An aspect of the invention provides a kit for treating or preventing ocular angiogenesis or ocular fibrosis in a subject or cells from the subject, the kit including: a pharmaceutical composition that inhibits a galectin protein or portion thereof, such that the composition binds to the galectin protein and modulates a VEGF/VEGF receptor-2 pathway or a fibroblast growth factor (FGF) pathway, or the composition modulates expression of the galectin protein; instructions for use; and, a container. The pharmaceutical composition includes any of the compositions described herein.

An aspect of the invention provides a kit for treating or preventing ocular angiogenesis or ocular fibrosis in a subject or cells from the subject, the kit including: a pharmaceutical composition that inhibits a galectin protein or portion, such that the active compound binds to the galectin protein and modulates a VEGF/VEGF receptor-2 pathway or the composition modulates expression of the galectin protein; instructions for use; and, a container. The pharmaceutical composition includes any of the compositions described herein including for example a TD139 compound, or a Compound 32 compound.

The kit in various embodiments further includes an applicator or device for administering the composition, for example the applicator or the device is a syringe, a needle, a sprayer, a sponge, a gel, a strip, a tape, a bandage, a tray, a string, or a nanostructure.

The composition in various embodiments of the kit is selected from at least one of: a drug, a polymer, a protein, a peptide, a carbohydrate, a low molecular weight compound, an oligonucleotide, a polynucleotide, and a genetic material such as DNA or RNA. In a related embodiment, the composition is formulated with a pharmaceutically suitable carrier or a diluent.

The kit in various embodiments further includes a control for example AVASTIN™ (BEVACIZUMAB™), LUCENTIS™ (RANIBIZUMAB™), or EYLEA™ (AFLIBERCEPT™), such that the control specifically binds VEGF and inhibits VEGF-induced angiogenesis. In various embodiments, the control binds or inhibits FGF or an FGF pathway. In various embodiments of the kit, the pharmaceutical composition binds the galectin protein that is selected from the group of: galectin-1 protein, galectin-3 protein, galectin-7 protein, and galectin-8 protein.

An aspect of the invention provides a pharmaceutical composition for use in inhibiting a condition of ocular angiogenesis or ocular fibrosis, such that the composition includes a pharmaceutically suitable carrier or a diluent and an amount of the composition effective to inhibit or to modulate an activity of a galectin protein or portion thereof sufficient to inhibit the ocular angiogenesis or ocular fibrosis. In general, the carrier and the diluent are specifically selected to be compatible, non-toxic, and non-irritating to the eye of the subject.

In various embodiments of the pharmaceutical composition, the galectin protein is selected from the group of galectin-1, galectin-3, galectin-7, and galectin-8. The galectin-protein is derived for example from a human, a mouse, a rat, or a rabbit.

The pharmaceutical composition in various embodiments is selected from at least one of: a drug, a polymer, a protein, a peptide, a carbohydrate, a low molecular weight compound, an oligonucleotide, a polynucleotide, and a genetic material such as DNA or RNA. For example, the protein is an antibody that specifically binds an epitope of the galectin protein such that the antibody inhibits the activity of the galectin protein. In certain embodiments, the antibody is a chimeric antibody, a monoclonal antibody, a polyclonal antibody, or a portion thereof. For example the antibody specifically binds the galectin carbohydrate binding domain or portion thereof.

The pharmaceutical composition in various embodiments is effective to treat or prevent the condition associated with the ocular angiogenesis or ocular fibrosis, for example the disease or the condition is associated with excessive neovascularization. The pharmaceutical composition in various embodiments is effective to treat or prevent the condition selected from the group of: a trauma or injury to the eye or a portion thereof (e.g., retina, lens, and cornea); a surgery associated with scarring for example a glaucoma filtration surgery; neovascular glaucoma; a corneal injury; a corneal dystrophy, post-conjunctivitis scarring; pterygium, AMD for example wet AMD or dry AMD; conversion from dry to wet AMD; proliferative diabetic retinopathy; diabetic macular adema; and corneal neovascularization (trachoma).

In various embodiments, the pharmaceutical composition is effective to treat or prevent ocular fibrosis, or another type of fibrosis associated with the condition.

The pharmaceutical composition in various embodiments includes a compound having the following structure:

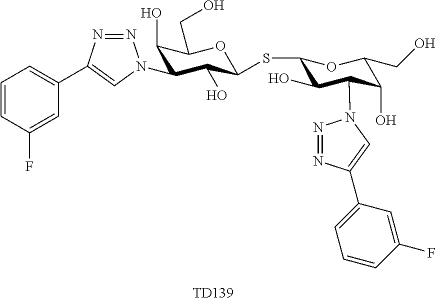

such that the compound inhibits the ocular angiogenesis condition in the subject.

The pharmaceutical composition in a related embodiment is a beta-galactoside that is derivatized or functionalized, for example, the composition has the following general formula:

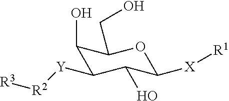

such that the configuration of the pyranose ring is D-galacto; X is selected from the group consisting of O, S, NH, CH₂, and NR⁴, or is a bond; Y is selected from the group consisting of NH, CH₂, and NR⁴, or is a bond; R¹ is selected from the group consisting of: c) a saccharide; d) hydrogen, an alkyl group, an alkenyl group, an aryl group, a heteroaryl group, and a heterocycle;
R² is selected from the group consisting of CO, SO₂, SO, PO, and PO₂; R³ is selected from the group consisting of: an alkyl group of at least 4 carbon atoms, an alkenyl group of at least 4 carbon atoms, an alkyl or alkenyl group of at least 4 carbon atoms substituted with a carboxy group, an alkyl group of at least 4 carbon atoms substituted with both a carboxy group and an amino group, and an alkyl group of at least 4 carbon atoms substituted with a halogen; a phenyl group, a phenyl group substituted with a carboxy group, a phenyl group substituted with at least one halogen, a phenyl group substituted with an alkoxy group, a phenyl group substituted with at least one halogen and at least one carboxy group, a phenyl group substituted with at least one halogen and at least one alkoxy group, a phenyl group substituted with a nitro group, a phenyl group substituted with a sulfo group, a phenyl group substituted with an amine group, a phenyl group substituted with a hydroxy group, a phenyl group substituted with a carbonyl group and a phenyl group substituted with a substituted carbonyl group; and a phenyl amino group; R⁴ is selected from the group consisting of hydrogen, an alkyl group, an alkenyl group, an aryl group, a heteroaryl group, and a heterocycle. In various embodiments the composition is metabolizable or non-metabolizable.

In various embodiments of the pharmaceutical composition, the saccharide R₁ is selected from the group consisting of glucose, mannose, galactose, N-acetylglucosamine, N-acetylgalactosamine, fucose, fructose, xylose, sialic acid, glucuronic acid, iduronic acid, a disaccharide or, an oligosaccharide comprising at least two of the above saccharides, and derivatives thereof.

In various embodiments of the pharmaceutical composition Y is NH; X is O; or the halogen is selected from the group consisting of F, Cl, Br and I.

The pharmaceutical composition in various embodiments is one selected from: methyl 2-acetamido-2-deoxy-4-O-(3-[3-carboxypropanamido]-3-deoxy-β-D-galactopyranosyl)-β-D-glucopyranoside; methyl 2-acetamido-2-deoxy-4-O-(3-[{Z}-3-carboxypropenamido]-3-deoxy-β-D-galactopyranosyl)-β-D-glucopyranoside; methyl 2-acetamido-2-deoxy-4-O-(3-benzamido-3-deoxy-β-D-galactopyranosyl)-β-D-glucopyranoside; methyl 2-acetamido-2-deoxy-4-O-(3-[2-carboxy-benzamido]-3-deoxy-β-D-galactopyranosyl)-β-D-glucopyranoside; methyl 2-acetamido-2-deoxy-4-O-(3-[4-methoxy-2,3,5,6-tetrafluorbenzamido]-3-deoxy-β-D-galactopyranosyl)-β-D-glucopyranoside; methyl 2-acetamido-2-deoxy-4-O-(3-[2-carboxy-3,4,5,6-tetrafluorbenzamido]-3-deoxy-β-D-galactopyranosyl)-β-D-glucopyranoside; methyl 2-acetamido-2-deoxy-4-O-(3-methanesulfonamido-3-deoxy-β-D-galactopyranosyl)-β-D-glucopyranoside; methyl 2-acetamido-2-deoxy-4-O-(3-[-4-nitrobenzenesulfonamido]-3-deoxy-β-D-galactopyranosyl)-β-D-glucopyranoside, methyl 2-acetamido-2-deoxy-4-O-(3-phenylaminocarbonylamino-3-deoxy-β-D-galactopyranosyl)-(3-D-glucopyranoside; methyl 2-acetamido-2-deoxy-4-O-(3-aminoacetamido-3-deoxy-β-D-galactopyranosyl)-β-D-glucopyranoside; and methyl 2-acetamido-2-deoxy-4-O-(-3-[{2S}-2-amino-3-carboxy-propanamido]-3-deoxy-β-D-galactopyranosyl)-f-D-glucopyranoside.

In various embodiments the pharmaceutical composition has the general formula:

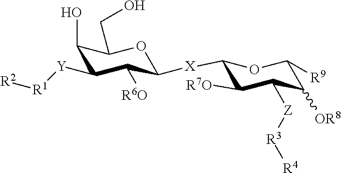

such that the configuration of one of the pyranose rings is β-D-galacto; X is selected from the group consisting of O, S, SO, SO₂, NH, CH₂, and NR⁵, Y is selected from the group consisting of O, S, NH, CH₂, and NR⁵, or is a bond; Z is selected from the group consisting of O, S, NH, CH₂, and NR⁵, or is a bond; R¹ and R³ are independently selected from the group consisting of CO, SO₂, SO, PO₂, PO, and CH₂ or is a bond; R² and R⁴ are independently selected from the group consisting of: an alkyl group of at least 4 carbons, an alkenyl group of at least 4 carbons, an alkyl group of at least 4 carbons substituted with a carboxy group, an alkenyl group of at least 4 carbons substituted with a carboxy group, an alkyl group of at least 4 carbons substituted with an amino group, an alkenyl group of at least 4 carbons substituted with an amino group, an alkyl group of at least 4 carbons substituted with both an amino and a carboxy group, an alkenyl group of at least 4 carbons substituted with both an amino and a carboxy group, and an alkyl group substituted with one or more halogens; a phenyl group substituted with at least one carboxy group, a phenyl group substituted with at least one halogen, a phenyl group substituted with at least one alkoxy group, a phenyl group substituted with at least one nitro group, a phenyl group substituted with at least one sulfo group, a phenyl group substituted with at least one amino group, a phenyl group substituted with at least one alkylamino group, a phenyl group substituted with at least one arylamino group, a phenyl group substituted with at least one dialkylamnino group, a phenyl group substituted with at least one hydroxy group, a phenyl group substituted with at least one carbonyl group and a phenyl group substituted with at least one substituted carbonyl group; a naphthyl group, a naphthyl group substituted with at least one carboxy group, a naphthyl group substituted with at least one halogen, a naphthyl group substituted with at least one alkoxy group, a naphthyl group substituted with at least one nitro group, a naphthyl group substituted with at least one sulfo group, a naphthyl group substituted with at least one amino group, a naphthyl group substituted with at least one alkylamino group, a naphthyl group substituted with at least one arylamino group, a naphthyl group substituted with at least one dialkylamnino group, a naphthyl group substituted with at least one hydroxy group, a naphthyl group substituted with at least one carbonyl group and a naphthyl group substituted with at least one substituted carbonyl group; a heteroaryl group, a heteroaryl group substituted with at least one carboxy group, a heteroaryl group substituted with at least one halogen, a heteroaryl group substituted with at least one alkoxy group, a heteroaryl group substituted with at least one nitro group, a heteroaryl group substituted with at least one sulfo group, a heteroaryl group substituted with at least one amino group, a heteroaryl group substituted with at least one alkylamino group, a heteroaryl group substituted with at least one dialkylamino group, a heteroaryl group substituted with at least one arylamino group, a heteroaryl group substituted with at least one hydroxy group, a heteroaryl group substituted with at least one carbonyl group and a heteroaryl group substituted with at least one substituted carbonyl group; R⁶ and R⁸ are independently selected from the group consisting of a hydrogen, an acyl group, an alkyl group, a benzyl group, and a saccharide; R⁷ is selected from the group consisting of a hydrogen, an acyl group, an alkyl group, and a benzyl group; or R⁹ is selected from the group consisting of a hydrogen, a methyl group. hydroxymethyl group, an acyloxymethyl group, an alkoxymethyl group, and a benzyloxymethyl group.

In various embodiments of the pharmaceutical composition Y is NH; Z is NH; X is S; R¹ is CO; R³ is CO; R² or R⁴ is an aromatic for example an aromatic ring; either of R⁶, R⁷, and R⁸ is hydrogen; or R⁹ is a hydroxymethyl group.

The pharmaceutical composition in various embodiment includes at least one selected from the group of: bis-(3-deoxy-3-benzamido-β-D-galactopyranosyl)sulfane (17), bis-(3-deoxy-3-(3-methoxybenzamido)-β-D-galactopyranosyl)sulfane; bis-(3-deoxy-(3,5-dimethoxybenzamido)-β-D-galactopyranosyl)sulfane; bis-(3-deoxy-3-(4-nitrobenzamido)-β-D-galactopyranosyl)sulfane; bis(3-deoxy-3-(2-naphthamido)-β-D-galactopyranosyl)sulfane; bis-(3-deoxy-3-(4-methoxybenzamido)-β-D-galactopyranosyl)sulfane; bis-(3-deoxy-3-(3-nitrobenzamido)-β-D-galactopyranosyl)sulfane; bis-(3-deoxy-3-[4-(dimethylamino)-benzamido]-β-D-galactopyranosyl)sulfane; bis-(3-deoxy-3-(4-methylbenzamido)-β-D-galactopyranosyl)sulfane; bis-(3-deoxy-3-(4-chlorobenzamido)-β-D-galactopyranosyl)sulfane; bis-(3-deoxy-3-(4-tert-butylbenzamido)-β-D-galactopyranosyl)sulfane; bis-(3-deoxy-3-(4-acetylbenzamido)-β-D-galactopyranosyl)sulfane; bis-(3-deoxy-3-[2-(3-carboxy)-naphthamido]-β-D-galactopyranosyl)sulfane; bis-[3-deoxy-3-(3,4-methylenedioxyl)benzamido]-β-D-galactopyranosyl)sulfane, bis-(3-deoxy-3-(4-methoxycarbonylbenzamido)-β-D-galactopyranosyl)sulfane; bis-(3-deoxy-3-(3-carboxybenzamido)-β-D-galactopyranosyl)sulfane; bis-(3-deoxy-3-(3-benzyloxy-5-hydroxy-benzamido)-β-D-galactopyranosyl)sulfane; bis-(3-deoxy-3-(3,5-dibenzyloxybenzamido)-β-D-galactopyranosyl)sulfane; bis-(3-deoxy-3-(3-benzyloxy-5-methoxy-benzamido)-β-D-galactopyranosyl)sulfane; bis-(3-deoxy-3-(3-benzyloxy-5-nonyloxy-benzamido)-β-D-galactopyranosyl)sulfane; bis-(3-deoxy-3-(3-hydroxy-5-methoxy-benzamido)-β-D-galactopyranosyl)-sulfane; bis-(3-deoxy-3-(3-hydroxy-5-nonyloxy-benzamido)-β-D-galactopyranosyl)sulfane, bis-(3-deoxy-3-[3-benzyloxy-5-(4-fluoro-benzyloxy)-benzamido]-β-D-galactopyranosyl)sulfane; bis-(3-deoxy-3-[3-methoxy-5-(4-methyl-benzyloxy)-benzamido]-β-D-galactopyranosyl)sulfane; or bis-(3-deoxy-3-(3-allyloxy-5-benzyloxy-benzamido)-β-D-galactopyranosyl)sulfane.

The pharmaceutical composition in various embodiments includes a 3-triaxolyl-galactoside, for example the composition comprises a general formula shown below:

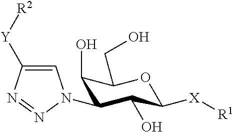

such that the configuration of the pyranose ring is D-galacto; X is selected from the group consisting of O, S, NH, CH₂, and NR⁴, or is a bond; Y is selected from the group consisting of CH₂, CO, SO₂, SO, PO₂ and PO, phenyl, or is a bond; R₁ is selected from the group consisting of: a saccharide; a substituted saccharide; D-galactose; substituted D-galactose; C3-[1,2,3]-triazol-1-yl-substituted D-galactose; hydrogen, an alkyl group, an alkenyl group, an aryl group, a heteroaryl group, and a heterocycle and derivatives thereof; and an amino group, a substituted amino group, an imino group, or a substituted imino group; and, R² is selected from the group consisting of; hydrogen, an amino group, a substituted amino group, an alkyl group, a substituted alkyl group, an alkenyl group, a substituted alkenyl group, an alkynyl group, a substituted alkynyl group, an alkoxy group, a substituted alkoxy group, an alkylamino group, a substituted alkylamino group, an arylamino group, a substituted arylamino group, an aryloxy group, a substituted aryloxy group, an aryl group, a substituted aryl group, a heteroaryl group, a substituted heteroaryl group, and a heterocycle, a substituted heterocycle.

The saccharide in various embodiments of the pharmaceutical composition is selected from the group consisting of glucose, mannose, galactose, N-acetylglucosamine, N-acetylgalactosamine, fucose, fructose, xylose, sialic acid, glucuronic acid, iduronic acid, galacturonic acid, a disaccharide or an oligosaccharide comprising at least two of the above saccharides, and derivatives thereof.

In various embodiments of the pharmaceutical composition Y is CO, SO₂, or a bond; R² is an amine or an aryl group; R¹ is galactose, glucose or N-acetylglucosamine; R¹ is substituted galactose, glucose or N-acetylglucosamine; R¹ is a C3-[1,2,3]-triazol-1-yl-substituted galactose; or X is O or S.

In various embodiments of the composition, R² is a substituted phenyl group wherein said substituent is one or more selected from the group consisting of halogen, alkoxy, alkyl, nitro, sulfo, amino, hydroxy or carbonyl group.

The pharmaceutical composition in various embodiments is formulated for ocular delivery. In various embodiments the composition is formulated for ocular delivery as an injection, an eye drop, or an ointment. For example the injection is at least one selected from the group consisting of: intra-vitreous injection, intra-ocular injection, subconjunctival injection, and subtenon injection.

The pharmaceutical composition is or includes in various embodiments selected from: methyl 3-deoxy-3-(1H-[1,2,3]-triazol-1-yl)-1-thio-β-D-galactopyranoside; methyl 3-deoxy-3-(4-propyl-1H-[1,2,3]-triazol-1-yl)-1-thio-β-D-galactopyranoside; methyl 3-(4-methoxycarbonyl-1H-[1,2,3]-triazol-1-yl)-3-deoxy-1-thio-β-D-galactopyranoside; methyl 3-deoxy-3-(4-(1-hydroxy-1-cyclohexyl)-1H-[1,2,3]-triazol-1-yl)-1-thio-β-D-galactopyranoside; methyl 3-deoxy-3-(4-phenyl-1H-[1,2,3]-triazol 1-yl)-1-thio-β-D-galactopyranoside; methyl 3-deoxy-3-(4-p-tolylsulfonyl-1H-[1,2,3]-triazol 1-yl)-1-thio-β-D-gal-actopyranoside; methyl 3-(4-methylaminocarbonyl-1H-[1,2,3]-triazol-1-yl)-3-deoxy-1-thio-β-D-galactopyranoside; methyl 3-(4-butylaminocarbonyl-1H-[1,2,3]-triazol-1-yl)-3-deoxy-1-thio-β-D-galactopyranoside; methyl 3-(4-benzylaminocarbonyl-1H-[1,2,3]-triazol-1-yl)-3-deoxy-1-thio-β-D-galactopyranoside; methyl 3-{4-(3-hyroxyprop-1-ylaminocarbonyl)-1H-[1,2,3]-triazol-1-yl}-3-deoxy-1-thio-β-D-galactopyranoside; methyl 3-{4-[2-(N-morpholino)-ethylaminocarbonyl]-1H-[1,2,3]-triazol-1-yl}-3-deoxy-1-thio-β-D-galactopyranoside; methyl 3-(4-methylaminocarbonyl-1H-[1,2,3]-triazol-1-yl)-3-deoxy-β-D-galactopyranosyl-(1→4)-2-acetamido-2-deoxy-β-D-glucopyranoside, bis-(3-deoxy-3-(4-(methylaminocarbonyl)-1H-[1,2,3]-triazol-1-yl)-β-D-galactopyranosyl)sulfane, methyl 3-deoxy-3-{4-(2-fluorophenyl)-1H-[1,2,3]-triazol-1-yl}-1-thio-β-D-galactopyranoside; methyl 3-deoxy-3-{4-(2-methoxyphenyl)-1H-[1,2,3]-triazol-1-yl}-1-thio-β-D-galactopyranoside; methyl 3-deoxy-3-{4-(3-methoxyphenyl)-1H-[1,2,3]-triazol-1-yl}-1-thio-β-D-galactopyranoside; methyl 3 deoxy-3-{4-(4-methoxyphenyl)-1H-[1,2,3]-triazol-1-yl}-1-thio-β-D-galactopyranoside; methyl 3-deoxy-3-{4-(3,5-dimethoxyphenyl)-1H-[1,2,3]-triazol-1-yl}-1-thio-β-D-galactopyranoside; methyl 3-deoxy-3-{4-(1-naphthyl)-1H-[1,2,3]-triazol-1-yl}-1-thio-β-D-galactopyranoside; methyl 3-deoxy-3-{4-(2-naphthyl)-1H-[1,2,3]-triazol-1-yl}-1-thio-β-D-galactopyranoside; methyl 3-deoxy-3-{4-(2-pyridyl)-1H-[1,2,3]-triazol-1-yl}-1-thio-β-D-galactopyranoside; methyl 3-deoxy-3-{4-(3-pyridyl)-1H-[1,2,3]-triazol-1-yl}-1-thio-β-D-galactopyranoside; methyl 3-deoxy-3-{4-(4-pyridyl)-1H-[1,2,3]-triazol-1-yl}-1-thio-β-D-galactopyranoside; O-{3-deoxy-3-[4-phenyl-[1H-[1,2,3]-triazol-1-yl]-β-D-galactopyranosy-l}-3-indol-carbaldoxim; O-{3-deoxy-3-[4-(methylaminocarbonyl)-1H-[1,2,3]-triazol-1-yl]-β-D-galactopyranosyl}-3-indol-carbaldoxim; O-{3-deoxy-3-[4-phenyl-[1H-[1,2,3]-triazol-1-yl]-β-D-galactopyranosy-l}-(2-hydroxy-5-nitro-phenyl)-carbaldoxim; O-{3-deoxy-3-[4-(methylaminocarbonyl)-1H-[1,2,3]-triazol-1-yl]-β-D-galactopyranosyl}-(2-hydroxy-5-nitro-phenyl)-carbaldoxim; O-{3-deoxy-3-[4-phenyl-[1H-[1,2,3]-triazol-1-yl]-β-galactopyranosy-l}-(2,5-dihydroxyphenyl)-carbaldoxim; O-{3-deoxy-3-[4-(methylaminocarbonyl)-1H-[1,2,3]-triazol-1-yl]-β-D-galactopyranosyl}-(2,5-dihydroxyphenyl)-carbaldoxim; O-{3-deoxy-3-[4-phenyl-[H-[1,2,3]-triazol-1-yl]-β-D-galactopyranosy-l}-1-naphthyl-carbaldoxim; or O-{3-deoxy-3-[4-(methylaminocarbonyl)-1H-[1,2,3]-triazol-1-yl]-β-D-galactopyranosyl}-1-naphthyl-carbaldoxim.

In various embodiments, the pharmaceutical composition includes a thiodigalactoside, for example the composition has a general formula shown below:

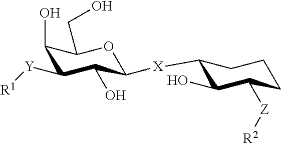

such that the configuration of the pyranose ring is D-galacto; X is selected from the group consisting of O, S, and SO; Y and Z are independently selected from being CONH or a 1H-1,2,3-triazole ring; R¹ and R² are independently selected from the group consisting of: an alkyl group of at least 4 carbons, an alkenyl group of at least 4 carbons, an alkynyl group of at least 4 carbons; a carbamoyl group, a carbamoyl group substituted with an alkyl group, a carbamoyl group substituted with an alkenyl group, a carbamoyl group substituted with an alkynyl group, a carbamoyl group substituted with an aryl group, a carbamoyl group substituted with an substituted alkyl group, and a carbamoyl group substituted with an substituted aryl group; a phenyl group substituted with at least one carboxy group, a phenyl group substituted with at least one halogen, a phenyl group substituted with at least one alkyl group, a phenyl group substituted with at least one alkoxy group, a phenyl group substituted with at least one trifluoromethyl group; a phenyl group substituted with at least one trifluoromethoxy group, a phenyl group substituted with at least one sulfo group, a phenyl group substituted with at least one hydroxy group, a phenyl group substituted with at least one carbonyl group, and a phenyl group substituted with at least one substituted carbonyl group; a naphthyl group, a naphthyl group substituted with at least one carboxy group, a naphthyl group substituted with at least one halogen, a naphthyl group substituted with at least one alkyl group, a naphthyl group substituted with at least one alkoxy group, a naphthyl group substituted with at least one sulfo group, a naphthyl group substituted with at least one hydroxy group, a naphthyl group substituted with at least one carbonyl group, and a naphthyl group substituted with at least one substituted carbonyl group; a heteroaryl group, a heteroaryl group substituted with at least one carboxy group, a heteroaryl group substituted with at least one halogen, a heteroaryl group substituted with at least one alkoxy group, a heteroaryl group substituted with at least one sulfo group, a heteroaryl group substituted with at least one arylamino group, a heteroaryl group substituted with at least one hydroxy group, a heteroaryl group substituted with at least one halogen, a heteroaryl group substituted with at least one carbonyl group, and a heteroaryl group substituted with at least one substituted carbonyl group; and a thienyl group, a thienyl group substituted with at least one carboxy group, a thienyl group substituted with at least one halogen, a thienyl thienyl group substituted with at least one alkoxy group, a thienyl group substituted with at least one sulfo group, a thienyl group substituted with at least one arylamino group, a thienyl group substituted with at least one hydroxy group, a thienyl group substituted with at least one halogen, a thienyl group substituted with at least one carbonyl group, and a thienyl group substituted with at least one substituted carbonyl group.

In various embodiments of the pharmaceutical composition, Y is CONH for example the CONH group is linked via the N atom to the pyranose ring; Z is CONH for example the CONH group is linked via the N atom to the cyclohexane; Y is a 1H-1,2,3-triazole ring for example linked via the N1 atom to the pyranose ring; R¹ is linked to the C4 atom of the 1H-1,2,3-triazole ring; Z is a 1H-1,2,3-triazole ring for example the 1H-1,2,3-triazole ring is linked via the N1 atom to the cyclohexane; or R² is linked to the C4 atom of the 1H-1,2,3-triazole ring.

In various embodiments R¹ and R² of the pharmaceutical composition are independently selected from the group consisting of a carbamoyl group, an alkylated carbamoyl group, an alkenylated carbamoyl group, an arylated carbamoyl group, a phenyl group, a substituted phenyl group, a halogenated phenyl group, a fluorinated phenyl group, a chlorinated phenyl group, a brominated phenyl group, an alkylated phenyl group, an alkenylated phenyl group, a trifluoromethylated phenyl group, a methoxylated phenyl group, a trifluoromethoxylated phenyl group, a naphthyl group, a substituted naphthyl group, a heteroaryl group, a substituted heteroaryl group, a thienyl group, and a substituted thienyl group.

R¹ in various embodiments of the pharmaceutical composition is an alkylated carbamoyl group, a fluorinated phenyl group, or a thienyl group.

In various embodiments of the pharmaceutical composition, R² is an alkylated carbamoyl group, a fluorinated phenyl group, or a thienyl group, or X is O or S.

The pharmaceutical composition in various embodiments is selected from the group consisting of: ((1R,2R,3S)-2-hydroxy-3-(4-(N-(1-propyl)-carbamoyl)-1H-1,2,3-triazol-1-yl)cyclohexyl) 3-deoxy-(3-(4-(N-(1-propyl)-carbamoyl)-1H-1,2,3-triazol-1-yl))-β-D-galactopyranoside; ((1R,2R,3S)-2-hydroxy-3-(4-(2-fluorophenyl)-1H-1,2,3-triazol-1-yl)-cyclohexyl) 3-deoxy-3-(4-(2-fluorophenyl)-1H-1,2,3-triazol-1-yl)-1-thio-β-D-galactopyranoside; ((1R,2R,3S)-2-hydroxy-3-(4-(3-fluorophenyl)-1H-1,2,3-triazol-1-yl)-cyclohexyl) 3-deoxy-3-(4-(3-fluorophenyl)-1H-1,2,3-triazol-1-yl)-1-thio-β-D-galactopyranoside; ((1R,2R,3S)-2-hydroxy-3-(4-(4-fluorophenyl)-1H-1,2,3-triazol-1-yl)-cyclohexyl) 3-deoxy-3-(4-(4-fluorophenyl)-1H-1,2,3-triazol-1-yl)-1-thio-β-D-galactopyranoside; (1R,2R,3S)-2-hydroxy-3-(4-(3-thienyl)-1H-1,2,3-triazol-1-yl)-cyclohexyl) 3-deoxy-3-(4-(3-thienyl)-1H-1,2,3-triazol-1-yl)-1-thio-β-D-galactopyranoside; (1R,2R,3S)-2-hydroxy-3-(4-(N-(1-propyl)-carbamoyl)-1H-1,2,3-triazol-1-yl)-cyclohexyl) 3-deoxy-3-(4-(N-(1-propyl)-carbamoyl)-1H-1,2,3-triazol-1-yl)-1-thio-β-D-galactopyranoside, and (1R,2R,3S)-2-hydroxy-3-(4-chlorobenzamido)-cyclohexyl) 3-deoxy-3-(4-chlorobenzamido)-1-thio-β-D-galactopyranoside.

In various embodiments, the pharmaceutical composition includes a digalactoside, for example the composition includes a general formula (13)

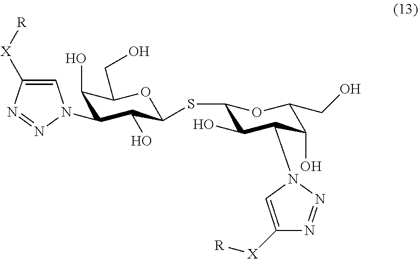

wherein the configuration of at least one of the pyranose rings is D-galacto; X is a bond; R is a phenyl group, which is substituted in any position with one or more substituents selected from the group consisting of methyl, ethyl, isopropyl, tert-butyl, fluoro, chloro, bromo, and trifluoromethyl or R is a thienyl group.

In various embodiments of the composition R is a phenyl group which is substituted in any position with one or more substituents selected from the group consisting of fluoro, chloro, and bromo. For instance R is a phenyl group which is substituted in any position with one or more substituents selected from fluoro. Typically, the configuration of both pyranose rings is D-galacto.

An aspect of the invention provides a method for treating or preventing an ocular angiogenesis or ocular fibrosis condition in a subject, the method including administering a therapeutically effective amount of at least one composition to the subject, such that the composition includes an inhibitor of a galectin protein or a portion thereof. The subject in various embodiments is a mammal.

The condition in various embodiments of the method is selected from the group of: a surgery associated with scarring for example a glaucoma filtration surgery; neovascular glaucoma; a corneal injury; post-conjunctivitis scarring; pterygium, age related-macular degeneration (AMD) for example wet AMD or dry AMD; conversion from dry to wet AMD; proliferative diabetic retinopathy; diabetic macular adema; and corneal neovascularization (trachoma).

In various embodiments of the method, the galectin protein is selected from the group of: galectin-1 protein, galectin-3 protein, galectin-7 protein, and galectin-8, and such that the composition is selected from at least one of: a drug, a polymer, a protein, a peptide, a carbohydrate, a low molecular weight compound, an oligonucleotide, a polynucleotide, and a genetic material such as DNA or RNA.

The method in various embodiments further involves observing reduction in a parameter or an indicator of the disease or the condition, for example the parameter or the indicator is a marker.

In various embodiments, the method further includes, prior to administering, engineering the composition, such that the composition binds to the galectin protein and modulates a VEGF/VEGF receptor-2 pathway, or the composition modulates expression of the galectin protein. In a related embodiment the inhibitor modulates a FGF and inhibits FGF-induced angiogenesis.

An aspect of the invention provides a method for treating or preventing a condition ocular angiogenesis or ocular fibrosis in a subject, the method involving administering a therapeutically effective amount of at least one composition to the subject, such that the composition comprises an inhibitor of a galectin protein or a portion thereof, such that the administering comprises contacting the subject with any of the pharmaceutical compositions described herein. In various embodiments, the subject is a mammal.

In various embodiments, the method further involves observing or detecting a reduction in the fibrosis.

Administering the composition in various embodiments of the method includes contacting the subject or tissue of the subject with a dose of at least: about 0.01 nanograms (ng) to about 1 ng, about 1 ng to about 10 ng, about 10 ng to about 20 ng, about 20 ng to about 30 ng, about 30 ng to about 40 ng, about 40 ng to about 50 ng, about 50 ng to about 100 ng, 100 ng to about 200 ng, 200 ng to about 300 ng, about 300 ng to about 400 ng, about 400 ng to about 600 ng, about 600 ng to about 800 ng, about 1 microgram (μg) to about 5 μg, about 5 μg to about 20 μg, about 20 μg to about 40 μg, about 40 μg to about 60 μg, about 60 μg to about 80 μg, about 80 μg to about 100 μg, about 100 μg to about 200 μg, about 200 μg to about 300 μg, and about 300 μg to about 400 μg.

The method in various embodiments further involves prior to administering, formulating the composition for an ocular delivery selected from the group of: an injection, an eye drop, a patch, an ointment, a gel, or a spray.

Administering the composition in various embodiments of the method includes injecting the composition for example intra-ocularly, intravitreally, corneally, conjunctively, or tenonly. For example, the injection is administered to a mucosal layer of the subject, or into an external layer of the eye.

An aspect of the invention provides a kit for treating or preventing ocular angiogenesis or ocuolar fibrosis condition in a subject or cells from the subject, the kit including: a pharmaceutical composition that inhibits a galectin protein or portion, such that the composition binds to the galectin protein and modulates a VEGF/VEGF receptor-2 pathway or the composition modulates expression of the galectin protein; instructions for use; and, a container. For example the pharmaceutical composition includes TD139, Compound 32, or an analog or derivative thereof.

An aspect of the invention provides a kit for treating or preventing a condition of ocular angiogenesis or ocular fibrosis in a subject or cells from the subject, the kit comprising: a pharmaceutical composition that inhibits a galectin protein or portion, such that the composition binds to the galectin protein and modulates a VEGF/VEGF receptor-2 pathway or the composition modulates expression of the galectin protein, such that the pharmaceutical composition is any of the compositions described herein; instructions for use; and, a container. For example the pharmaceutical composition includes at least one galectin inhibitor such as TD139, Compound 32, or an analog or derivative thereof.

The kit in various embodiments further includes an applicator or device for administering the composition, for example the applicator or the device is a syringe, a needle, a sprayer, a sponge, a gel, a strip, a tape, a bandage, a tray, a string, or a nanostructure.

The composition in various embodiments of the kit is selected from at least one of: a drug, a polymer, a protein, a peptide, a carbohydrate, a low molecular weight compound, an oligonucleotide, a polynucleotide, and a genetic material such as DNA or RNA. In various embodiments of the kit, the composition is formulated with a pharmaceutically suitable carrier or a diluent. In a related embodiment, the kit further includes a control for example AVASTIN™ (BEVACIZUMAB™), LUCENTIS™ (RANIBIZUMAB™), or EYLEA™ (AFLIBERCEPT™). The control in various embodiments is used with control subjects or samples to bind to VEGF and inhibit the ocular angiogenesis, or alternatively is used in combination with the pharmaceutical composition to inhibit the ocular angiogenesis. In a related embodiment of the kit, the AVASTIN™ specifically binds VEGF and inhibits VEGF-induced angiogenesis. In various embodiments, the control binds or inhibits a FGF or a FGF pathway. In various embodiments of the kit, the composition specifically binds and inhibits the galectin protein selected from the group of: galectin-1 protein, galectin-3 protein, galectin-7 protein, and galectin-8.

An aspect of the invention provides a product including: a pharmaceutical composition described herein that inhibits a galectin protein or portion thereof and is effective to inhibit or modulate ocular angiogenesis or ocular fibrosis; and an anti-angiogenesis agent or an anti-fibrosis agent, wherein the product is effective for use in anti-angiogenesis or anti-fibrosis therapy. In various embodiments, the product is a combined preparation for simultaneous, separate or sequential administration. For example, the anti-angiogenesis agent is at least one of AVASTIN™ (BEVACIZUMAB™), LUCENTIS™ (RANIBIZUMAB™), and EYLEA™ (AFLIBERCEPT™).

An aspect of the invention provides a method of inhibiting or modulating ocular angiogenesis or ocular fibrosis using the product described above, the method including administering at least one pharmaceutical composition described herein and another anti-angiogenesis agent. For example, the composition and/or the other anti-angiogenesis agent modulates expression or activity of a molecule in an angiogenesis signaling pathway. In various embodiments, the composition and/or the other anti-angiogenesis agent is a drug, protein, peptide, carbohydrate, or small molecule. In various embodiments, the composition and/or the other angiogenesis agent includes an inhibitor of a kinase receptor or an inhibitor of a tyrosine kinase receptor. In various embodiments the receptor is selected from a VEGF receptor (VEGFR), a platelet-derived growth factor receptor (PDGFR), an angiopoietin receptor (e.g., a Tie-1 and a Tie-2), a fibroblast growth factor receptor (FGFR), or an endothelial growth factor receptor (EGFR). In various embodiments, the other angiogenesis agent is AVASTIN™ (BEVACIZUMAB™), LUCENTIS™ (RANIBIZUMAB™), or EYLEA™ (AFLIBERCEPT™).

### BRIEF DESCRIPTION OF THE DRAWINGS

FIG. 1 depicts the amino acid sequence and composition of human galectin-3 protein (Accession No. BAA22164 in GenBank, SEQ ID NO: 1).

FIG. 2 depicts the amino acid sequence and composition of human galectin-7 protein (Accession No. 155469 in GenBank, SEQ ID NO: 2).

FIG. 3 depicts a CLUSTAL W alignment of the amino acid sequence of human galectin-3 protein (SEQ ID NO: 1) with the amino acid sequences of rabbit galectin-3 protein (Accession No. JC4300 in GenBank), chicken galectin-3 protein (Accession No. AAB02856 in GenBank), and hamster galectin-3 protein (Accession No. CAA55479 in GenBank). The first (upper) sequence in the figure is amino acids 1 to 250 of human galectin-3 protein (SEQ ID NO: 1), the second sequence in the figure is amino acids 1 to 245 of hamster galectin-3 protein, the third sequence in the figure is amino acids 1 to 242 of rabbit galectin-3 protein, and the fourth (lower) sequence in the figure is amino acids 1 to 262 of chicken galectin-3 protein.

FIG. 4 depicts a CLUSTAL W alignment of the amino acid sequence of human galectin-7 (SEQ ID NO: 2) with the amino acid sequences of rat galectin-7 protein (Accession No. P97590 in GenBank) and mouse galectin-7 protein (Accession No. 054974 in GenBank). The first (upper) sequence in the figure is amino acids 1 to 136 of rat galectin-7 protein, the second sequence in the figure is amino acids 1 to 136 of mouse galectin-7 protein, and the third (lower) sequence in the figure is amino acids 1 to 136 of human galectin-7 protein (SEQ ID NO: 2).

FIG. 5 is a summary of the results of a PROSITE scan of human galectin-3 protein (SEQ ID NO: 1).

FIG. 6 is a summary of the results of a PROSITE scan of human galectin-7 protein (SEQ ID NO: 2).

FIG. 7 depicts an alignment of the galactoside-binding domain of human galectin-3 protein with a consensus amino acid sequence (PF00337) derived from a hidden Markov model (HMM) from PFAM. The upper sequence is the consensus amino acid sequence (PF00337, SEQ ID NO: 3), while the lower amino acid sequence corresponds to amino acids 117 to 247 of SEQ ID NO: 1.

FIG. 8 depicts an alignment of the galactoside-binding domain of human galectin-7 protein with a consensus amino acid sequence (PF00337) derived from a hidden Markov model (HMM) from PFAM. The upper sequence is the consensus amino acid sequence (PF00337, SEQ ID NO: 3), while the lower amino acid sequence corresponds to amino acids 5 to 135 of SEQ ID NO: 2.

FIG. 9A and FIG. 9B are a bar graph and photomicrographs showing that TD139 galectin-3 inhibitor substantially reduced vascular endothelial growth factor (VEGF)-induced capillary tube formation in human umbilical vein endothelial cells (HUVEC).

FIG. 9A is a bar graph showing fold change of capillary tube formation in HUVEC compared to control HUVEC (ordinate) as a function of treatment (abscissa) with either 25 ng/mL of VEGF; 25 ng/mL of VEGF and ten micromolar TD139, or 25 ng/mL of VEGF and 50 micromolar TD139. Control HUVEC were not treated with VEGF nor TD139. Decreased capillary formation was observed for HUVEC treated with 25 ng/mL of VEGF and 10 micromolar TD139 and for HUVEC treated with 25 ng/mL of VEGF and 50 micromolar TD139 compared to HUVEC treated with 25 ng/mL of VEGF only.

FIG. 9B data is a set of photomicrographs of HUVEC treated with either: 25 ng/mL of VEGF (left); 25 ng/mL of VEGF and ten micromolar TD139 (middle); or 25 ng/mL of VEGF and 50 micromolar TD139 (right). The photomicrographs show extensive capullar tube formation for HUVEC contacted with VEGF compared to HUVEC contacted with a VEGF and TD139 galectin-3 protein inhibitor. Reduced capillary tubule formation was observed in HUVEC treated with the mixture of 25 ng/mL of VEGF and 50 micromolar TD139 (right) compared to HUVEC treated with the mixture of 25 ng/mL of VEGF and 10 micromolar TD139.

FIG. 10A-B are a bar graph and photomicrographs showing that a galectin-3 inhibitor TD139 substantially reduced VEGF-induced capillary sprouting in HUVEC an in vitro angiogenesis/sprouting assay.

FIG. 10A is a bar graph showing cumulative length of all capillary-like sprouts originating from the central plain of an individual spheroid (ordinate) of HUVEC as a function of treatment (abscissa) with either: 25 ng/mL of VEGF; 25 ng/mL of VEGF and ten micromolar TD139 galectin-3 protein inhibitor; or 25 ng/mL of VEGF and 50 micromolar TD139 galectin-3 protein inhibitor. Control HUVEC were not treated with neither VEGF nor TD139. Data show that HUVEC treated with VEGF only resulted in substantial sprouting length compared to control cells. Spherioid length was observed to decrease for HUVEC treated with 25 ng/mL of VEGF and 10 micromolar TD139, or 25 ng/mL of VEGF and 50 micromolar TD139. The greatest reduction in the VEGF-induced increase in sprouting length was observed for HUVEC treated with 50 micromolar TD139.

FIG. 10B data is a set of photomicrographs showing spheroids of HUVEC treated with either: 25 ng/mL of VEGF (top row right); 25 ng/mL of VEGF and ten micromolar TD139 galectin-3 protein inhibitor (bottom row left); or 25 ng/mL of VEGF and 50 micromolar TD139 galectin-3 protein inhibitor (bottom row right). Control HUVEC were not treated with neither VEGF nor TD139 (FIG. 10B top row left). The photomicrographs show that sprouting and length of sprouting for HUVEC contacted with VEGF compared to control cells. The VEGF-induced sprouting was reduced in HUVEC contacted with a mixture of VEGF and TD139. Reduced capillary tubule formation was observed in human endothelial cells treated with the mixture of 25 ng/mL of VEGF and 50 micromolar TD139 (right) compared to human endothelial cells treated with the mixture of 25 ng/mL of VEGF and 10 micromolar TD139 compared to cells treated with 25 ng/mL of VEGF only.

FIG. 11A, FIG. 11B and FIG. 11C are a drawing, a set of photomicrographs, and a graph showing that TD139 has an anti-neovascularization effect in vivo. Neovascularization was induced in mouse corneas by cauterizing the eye with silver nitrate.

FIG. 11A is a drawing of an experimental regimen for drug administration in murine subjects strain C57B/6L. Subjects were administered either ten microliters (μl) of TD139 (325 ng) in PBS containing 0.5% DMSO, or 10 μl of vehicle only (PBS containing 0.5% DMSO) by sub-conjunctival injection at each of day zero, day two and day four. In addition, a 10 μl of vehicle alone or 50 μM TD139 was applied once every day by eye drop. Silver nitrate was applied on day zero to cauterize the eyes of the subjects. Mice were sacrificed on day five, and flat mounts of corneas were stained with anti-CD31 to visualize blood vessels. Data shown in FIG. 11 B-C were from subjects treated as described in FIG. 11A.

FIG. 11B is a photomicrograph of representative eyes (top row) and corneal flatmounts (bottom row) of C57B/6L subjects treated as described in FIG. 11A five days after cauterization. The representative photographs of corneal flat mounts shown were stained with anti-CD31. The photomicrographs show the ocular tissue of subjects treated either with TD139 (right column) or control subjects treated with PBS containing 0.5% DMSO as described in FIG. 11A. Magnification was ×100.

FIG. 11C is a graph showing percent area of blood vessels (ordinate) in the eye of subjects administered TD139 as in FIG. 11A. Control subjects were administered PBS containing 0.5% DMSO. The density of blood vessels covering the whole cornea was quantified by ImageJ. Data was analyzed with Student's t test. Blood vessels covered approximately 40% of the cornea in control subjects, and covered 28% of cornea in TD139-treated subjects. The neovascular response in eyes after cautery was significantly inhibited for subjects administered TD139 galectin inhibitor compared to control subjects.

FIG. 12 is a photomicrograph of a western blot showing that galectin-1 and galectin-3 interacted with VEGFR-2. HUVEC lysates were incubated with galectin-1 and galectin-3-agarose beads in the presence or absence of 0.1 M sucrose or lactose. Control samples contained only HUVEC lysates or beads. Bound material was eluted with Laemmli sample buffer, added to 4-20% SDS-PAGE gels, and electrophoresed. Gels were transferred onto membrane blots, which was then probed with anti-VEGFR-2 antibody. VEGFR-2 antibody was observed bound to the HUVEC lysate control and not to the bead only control. The VEGF-2 antibody shows that HUVEC bound to both galectin-1 displaying beads and to galectin-3 displaying beads. The binding of the VEGF-2 antibody to the galectin-1 and galectin-3 displaying beads was inhibited by presence of lectin competing saccharide, lactose, and was not inhibited not by noncompeting saccharide sucrose. Thus, the interaction between VEGFR-2 and galectin-1 and galectin-3 was observed herein to be carbohydrate-dependent.

FIG. 13A and FIG. 13B are a set of bar graphs showing that TD139 interfered with VEGF-A-induced endothelial cell sprouting and migration. TD139 significantly inhibited VEGF-A-induced sprouting and migration in a dose-dependent manner. HPF indicates high-power field. Data are plotted as mean±SEM and analyzed with one-way ANOVA. *P<0.05 vs control; ***P<0.001 vs control; ^{###}P<0.001 vs VEGF-A.

FIG. 13A is a bar graph showing sprout length (ordinate) of HUVEC cells treated with (abscissa) VEGF-A (100 ng/mL) with or without TD139 (at each of 0.01 μM, 0.1 μM, 1 μM, 5 μM, or 10 μM) and controls with neither or TD139 alone (10 μM). HUVEC spheroids were prepared and seeded into type I collagen gels. Polymerized collagen was overlaid with 100 μl of EBM-2 and 25 ng/mL VEGF in EBM-2 supplemented either with 20 μM TD139 or 50 μM TD139. Control collagen samples were treated with 100 μl of EBM-2 and 25 ng/mL VEGF in EBM-2. After a six hour pre-treatment, gels were treated: VEGF-A (100 ng/ml) only, VEGF-A (100 ng/ml) and TD139 (0.01 μM, 0.1 μM, 1 μM, 5 μM, or 10 μM), or TD139 (10 μM) only. After a 24 hour incubation, spheroids were stained with calcein AM dye and photographed by a fluorescent microscope. Cumulative sprout lengths for HUVEC in gels were quantified by ImageJ. It was observed that contacting HUVEC with TD139 either 0.1 μM, 1 μM, 5 μM or 10 μM significantly inhibited VEGF-A-induced sprouting in a dose-dependent manner.

FIG. 13B is a bar graph showing migration distance (migration index, ordinate) of HUVEC cells treated with (abscissa) VEGF-A with or without VEGF-A as in FIG. 13A. HUVECs were serum starved overnight, detached with proteolytic and collagenolytic enzyme product Accutase™ (Millipore Inc.), resuspended in 1% FBS/M199 and then added to the upper chamber of a migration testing system. The bottom chamber of the system was filled with VEGF-A (100 ng/mL) in the presence or absence of varying concentrations of TD139 (1 μM, 10 μM, 100 μM, or 1000 μM) in 1% FBS/M199. After three hours of incubation, HUVECs that migrated to lower side of the membrane were counted. It was observed that presence of TD139 at as low as 1 nM concentration significantly abrogated VEGF-A-induced chemotaxis, and at greater concentrations. TD139 significantly further inhibited VEGF-A-induced migration in a dose-dependent manner.

FIG. 14A and FIG. 14B a set of bar graphs showing that TD139 is nontoxic to HUVEC. HUVECs were incubated in 1% FBS/M199 overnight and then treated with either 0.1% DMSO, or with 0.1% Triton™ X-100, or with 5 μM TD139 for three hours. The HUVEC were then incubated with either calcein AM dye or WST-1, a tetrazolium salt. Calcein AM and WST-1 are indicators of cell proliferation and cell viability. Fluorescence signals were detected by a spectrophotometer. HUVEC viability was not altered after TD139 treatment, indicating that inhibition of VEGF-A-induced functions by TD139 galectin inhibitor was not due to alteration of cell viability. The positive control Triton X-100 eliminated viability, data are plotted with mean±SEM and analyzed with one-way ANOVA. ***P<0.001 vs control.

FIG. 14A is a bar graph showing calcein AM fluorescence (ordinate) of HUVEC incubated in 1% FBS/M199 overnight and then treated with either 0.1% DMSO, 0.1% Triton™ X-100, or 5 μM TD139 for three hours (abscissa). Control cells were not treated with either DMSO, Triton™ X-100 or TD139. The treated cells were incubated with calcein AM for 30 minutes. Data show comparable calcein AM fluorescence for cells TD139 cells compared to cells treated with DMSO and control cells. Cells treated with Triton™ X-100 showed reduced fluorescence compared to cells treated with TD139.

FIG. 14B is a bar graph showing WST-1 fluorescence (ordinate) of HUVEC incubated in 1% FBS/M199 overnight and then treated with either 0.1% DMSO, 0.1% Triton™ X-100, or 5 μM TD139 for three hours (abscissa). Control cells were not treated with either DMSO, Triton™ X-100 or TD129. WST-1 was added to the treated cells and incubated for two hours. Data show comparable cell viability for cells TD139 cells compared to cells treated with DMSO and control cells. Cells treated with Triton™ X-100 showed reduced WST-1 fluorescence compared to cells treated with TD139.

FIG. 15 is a photograph of Western blots showing that VEGFR2 expression in cells was not affected by TD139 treatment. HUVECs were incubated in 1% FBS/M199 overnight and then treated with 0.1% DMSO and 10 μM TD139 for 6 hours. Cell lysates were analyzed by electrophoresis in 4-20% SDS-PAGE gels. The gels were transferred to western blot membranes, and were probed with anti-VEGFR-2 antibody (FIG. 15 top row) or β-actin antibody (FIG. 15 bottom row). VEGFR2 expression level was observed to be similar in both control and TD139-treated cells. Treatment of the endothelial cells with TD139 galectin inhibitor did not reduce total VEGFR2 expression.

FIGS. 16A-16C are a bar graph and a photomicrographs showing that galectin-3 inhibitor Compound 32 inhibited corneal vascularization in vivo. Corneas of 8-week-old C57BL/6 mice were cauterized with silver nitrate for five seconds to induce neovascularization. Ten μl of Compound 32 galectin-3 inhibitor (100 μM in 0.5% dimethyl sulfoxide, DMSO) was subconjunctivally injected into the corneas at day zero and days two, four and six afterwards. Control subjects were subconjunctively injected with ten μl of DMSO vehicle only (0.5% DMSO in PBS).

Subjects injected with Compound 32 were administered daily with eye drops (5 μl) of Compound 32, and control subjects were administered daily eyedrops (5 μl) of DMSO vehicle. At day seven corneas were excised and blood vessels and lymphatic vessels in the corneas were visualized and quantified. Combined data from two independent results are shown in FIG. 16A bar graph. Representative images are shown in FIG. 16B and FIG. 16C.

FIG. 16A a bar graph shows the percentage of blood vessel area (BV %; ordinate) in corneas in subjects subconjunctivally injected with ten μl of Compound 32 galectin-3 inhibitor (100 μM in 0.5% DMSO; abscissa). Control subjects were subconjunctively injected with ten μl of DMSO vehicle only (0.5% DMSO in PBS; abscissa). Eye drops (5 μl) of Compound 32 or DMSO vehicle were administered daily to the respective eyes. At day seven, mouse corneas were excised, and the area of blood vessels in the corneas was quantified. Data show a significant reduction blood vessel area in corneas of subjects injected with Compound 32 compared to control subjects injected DMSO vehicle only. The percentage area of blood vessels in the corneas of subjects injected with Compound 32 was 29%, and the percentage area of blood vessels in the corneas for control subjects was 42%. Data in FIG. 16A are plotted as Mean±SEM and analyzed using Student's t test.

FIGS. 16B and 16C photomicrographs are of representative corneas in subjects and subconjunctivally injected with ten μl of Compound 32 galectin-3 inhibitor (100 μM in 0.5% DMSO; FIG. 16C). Control subjects were subconjunctively injected with DMSO vehicle only (0.5% DMSO in PBS; FIG. 16B). The respective eyes were administered daily eye drops (five μl) of either Compound 32 or DMSO vehicle. The corneas were excised and visualized. Reduced number of blood vessels were observed in corneas of subjects injected with Compound 32 compared to control subjects injected DMSO vehicle only.

FIG. 17A-C are a set of photographs, a graph and a photograph of a western blot showing that TD139 inhibited corneal fibrosis. C57BJ/6 mice were anesthetized by intraperiteoneal injection of a mixture of ketamine and xylazine. Corneas of the subjects were topically anesthetized by applying an eye drop of proparacain. 1.5 μl drop of 0.15M sodium hydroxide was applied for one minute to the central cornea of each subject. Each eye was immediately washed extensively in PBS, and corneal epithelium and limbal epithelium were gently removed with an ALGEBRUSH™ optical device (Storz Ophthalmic Instruments, St. Louis, Mo.). Each cornea was covered with an antibiotic ointment. A volume of 10 μl of 50 mM TD139 was injected subconjunctivally each day for 14 days. Control subjects were injected subconjunctivally with control vehicle PBS. Corneas were visualized and analyzed for opacity, and protein markers for fibrosis were quantified as described below.

Corneal opacity of the eye was scored (arbitrary units) on days 7 and 14 post surgery using the following critera: 0 indicates that cornea is clear; 1 indicates area of opacity less than pupil; 2 indicates that area of opacity is bigger than pupil; 3 indicates that area of opacity is larger than 2/3 cornea area; and 4 indicates that entire cornea is opaque. Mice were sacrificed at day 14 post surgery using a mixture of ketamine and xylazine. Mouse corneas were dissected and lysed in ice-cold lysis buffer containing 5 mM NaF, 1 mM PMSF, 1 mM DTT, 20 mM HEPES, 1 mM EDTA, 400 mM NaCl, 1 mM EGTA, 0.1% NP-40, and a protease inhibitor cocktail product (Roche). Aliquots of corneal protein (20 μg) were loaded to each lane of a gel and transferred onto a nitrocellulose membrane. Blots were probed overnight at 4° C. with either a rabbit antibody specific for α-smooth muscle actin (10,000 dilution; Abcam Inc., Cambridge, England), or with a control antibody specific for β-actin. The blots were extensively washed and were incubated with goat anti rabbit IRDye 800CW antibody (1:10,000 dilution; LI-Cor Biosciences; Lincoln Nebr.). Membranes were scanned and quantified using an Odessey imaging system (Li-Cor Biosciences).

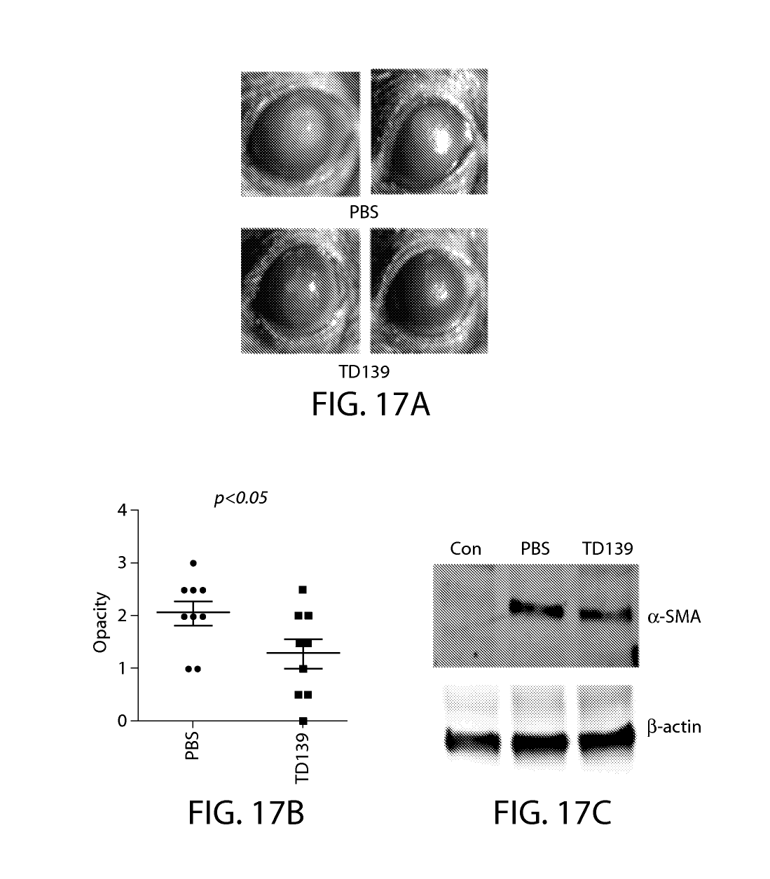

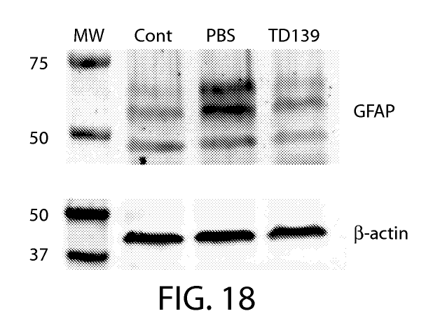

FIG. 17A is a set of photographs of representative corneas in subjects subconjunctivally injected with ten μl of 50 μM TD139 (FIG. 17A bottom row) and control subjects subconjunctively injected with vehicle only (FIG. 17B top row). It was observed that corneas from subjects administered TD139 had reduced amount/extent of corneal opacity compared to control subjects injected PBS vehicle only.

FIG. 17B is a bar graph showing amount of corneal opacity (arbitracy opacity score; ordinate) in corneas in subjects subconjunctivally injected with ten μl of TD139 (50 mM in PBS; abscissa). Control subjects were subconjunctively injected with ten μl of control vehicle only. The corneas were treated with sodium hydroxide, administered either TD139 or control PBS daily to the respective eyes for 14 days. At day fourteen, mouse corneas were excised, and the opacity in the corneas was quantified. Data show a significantly reduced extent of opacity in corneas of subjects injected with TD139 compared to control subjects injected PBS vehicle only. Subjects injected with TD139 had an area of opacity less than about the site of the pupil.

FIG. 17C is a photograph of a western blots showing that TD139 treatment reduced fibrosis in eyes having trauma. Corneas of subjects were contacted with sodium hydroxide, and were subconjunctively injected with an amount (10 μl) of 50 mM TD139 each day for 14 consecutive days. Control subjects were injected subconjunctively vehicle (PBS) only. Corneal cell lysates (20 μg) or a control β-actin material were analyzed by gel electrophoresis and western blot. The membranes were probed with mouse anti-SMA (smooth muscle actin) antibody (FIG. 17C top row), or were probed with a control antibody specific β-actin (FIG. 17C bottom row). SMA (smooth muscle actin) expression levels were in eyes injected with TD139 were observed to have been reduced by 30% compared to control eyes injected with PBS.

FIG. 18 is a set of photographs of western blots showing that TD139 treatment reduced retinal gliosis in eyes. Corneas of murine subjects were contacted with sodium hydroxide. A volume of TD139 10 μl of 50 mM solution was injected subconjunctively each day for 14 consecutive days. Control subjects were injected subconjunctively with PBS control vehicle only. Subjects were sacrificed by ketamine and xylazine on day 14 post surgery. Mouse corneas were dissected and lysed in ice-cold lysis buffer containing 5 mM NaF, 1 mM PMSF, 1 mM DTT, 20 mM HEPES, 1 mM EDTA, 400 mM NaCl, 1 mM EGTA, 0.1% NP-40 and a protease inhibitor cocktail product (Roche). Aliquots of retinal protein (30 μg) from lysates, or a control β-actin material were loaded to gels and transferred onto nitrocellulose membranes. Blots were probed overnight at 4° C. with rabbit anti mouse antibody specific for glial fibrillary acidic protein (GFAP; 50,000 dilution; Abcam Inc.; FIG. 18 top row), or with a control antibody specific for β-actin. GFAP is a marker for intermediate filaments indicative of retinal gliosis. Control blots were probed with rabbit antibody specific for β-actin (FIG. 18 bottom row). The probes were washed and were incubated with goat anti rabbit IRDye 800CW antibody (1:10,000 dilution; LI-Cor Biosciences). Membranes were scanned and quantified using an Odessey imaging system (Li-Cor Biosciences). It was observed that retinas from subjects injected with TD139 had reduced expression of GFAP compared to control subjects injected with PBS.

### DETAILED DESCRIPTION

Angiogenesis, or the growth of new blood vessels from preexisting vasculature, is involved in an array of normal processes such as embryonic development, wound healing, and reproductive function and in many pathological processes such as tumor growth and metastasis, ocular disorders and conditions such as Eales disease, neovascular glaucoma, and age-related macular degeneration, ischemia, diabetic microvascular disease, and rheumatoid arthritis (Markowska et al. 2011 J of Bio Chem Vol. 286 (34): 29913-29921; and Ocular Angiogenesis: Diseases, Mechanisms, and Therapeutics (Ophthalmology Research) 1st edition, Joyce Tombran-Tink and Colin J. Barnstable (editors) Human press published Apr. 1, 2006 pages 1-428, each of which is incorporated by reference herein in its entirety). Angiogenesis is promoted by loosening of endothelial cells from the basement membrane and periendothelial cells, promoting migration, proliferation, and ultimately formation of a new capillary lumen (Olsson et al. 2006 Rev. Mol. Cell Biol. 7: pages 359-371). Among a number of cytokine receptors and signaling factors, VEGF-A and its interaction with VEGF receptor 2 (VEGF-R2), have been identified as components of the major positive signal transduction pathway for both physiological and pathological angiogenesis. Binding of VEGF-A to VEGF-R2 promotes receptor dimerization, kinase activation, and autophosphorylation of multiple tyrosine residues within the dimeric complex. Autophosphorylation activates intracellular signaling pathways, such as the p42/44 MAP kinase pathway, that are involved in endothelial cell proliferation, migration, and survival.

Galectin-3, a member of the galectin family of galactoside binding proteins, promotes endothelial cell migration and capillary tubule formation in vitro and enhances vascularization in vivo (Markowska et al. 2011 J of Bio Chem Vol. 286 (34): 29913-29921, incorporated herein by reference in its entirety). VEGF-A-mediated angiogenesis is reduced in vitro by the addition of a dominant negative galectin-3, by a pan-galectin inhibitor (e.g., lactose), and by galectin-3 knockdown. Furthermore, VEGF-A induced angiogenesis was significantly reduced in Gal3−/− mice in vivo.

Primary open angle glaucoma (POAG) is a major blindness causing disease, characterized by elevated intraocular pressure due to an insufficient outflow of aqueous humor. The trabecular meshwork TM lining the aqueous outflow pathway modulates the aqueous outflow facility (Diskin et al. 2009 Glycobiology 19 (1): 29-37, incorporated by reference herein in its entirety). TM cell adhesion, cell-matrix interactions, and factors that influence Rho signaling in TM cells play a pivotal role in the regulation of aqueous outflow. Galectin-8 protein modulates adhesion and cytoskeletal arrangement of TM cells by binding to β1 integrins and inducing Rho signaling.

Galectin-8 protein has been shown to mediate TM cell adhesion and spreading as TM cells adhere to and spread on galectin-8 coated wells but not on galectin-1 coated wells or galectin-3 coated wells. Ibid. The adhesion of TM cells to galectin-8 coated wells was inhibited or prevented by administering a competing sugar, β-lactose, and was not inhibited by administering a noncompeting sugar such as sucrose. A trisaccharide, NeuAca2-3Galβ1-4GlcNAc, which binds specifically to the amino terminal carbohydrate recognition domain (CRD) of galectin-8 protein, inhibited spreading of TM cells to galectin-8 protein coated wells. In contrast, NeuAca2-6Galβ1-4GlcNAc which lacks affinity for galectin-8 had no effect on spreading of TM cells.

Affinity chromatography of cell extracts with a galectin-8 affinity column and binding experiments with plant lectins, Maackia amurensis and Sambucus nigra, revealed that a3β1, a5β1, and αvβ1 integrins are major counterreceptors of galectin-8 in TM cells and that TM cell β1 integrins carry predominantly α2-3-sialylated glycans, which are high-affinity ligands for Gal8 but not for Gal1 or Gal3. Galectin-8 protein modulates TM cell adhesion and spreading by interacting with α2-3-sialylated glycans on β1 integrins.

Galectins contain a carbohydrate recognition domain, CRD (Nilsson et al., U.S. patent publication number 2011/0130553 published Jun. 2, 2011, which is incorporated by reference herein in its entirety). The CRD is a tightly folded β-sandwich of about 130 amino acids (about 15 kDa) with the two characteristic features of a β-galactose binding site and sufficient similarity in a sequence motif of about seven amino acids, most of which (about six residues) make up the β-galactose binding site. Further, adjacent sites are required for tight binding of natural saccharides and different preferences of these confer on galectins different fine specificity for natural saccharides.

Completion of human, mouse and rat genome sequences reveal about 15 galectins and galectin-like proteins in one mammalian genome with slight variation between species (Leffler et al. 2004 Glycoconj. J. 19: 433-440; Houzelstein et al. 2004 Mol Biol Evol. 21(7): 1177-1187).

Galectin subunits contain one or two CRDs within a single peptide chain. The first category, mono-CRDs galectins, occurs as monomers or dimers (two types) in vertebrates. Galectin-1 is dimeric and galectin-3 is a monomer in solution and aggregates and becomes multimeric upon encounter with ligands (Leffler et al. 2004 Glycoconj. J. 19: 433-440; Ahmad et al. 2004 J. Biol. Chem. 279: 10841-10847).

Galectins are synthesized as cytosolic proteins on free ribosomes, without a signal peptide. An N-terminus of galectin protein is acetylated, a typical modification of cytosolic proteins, and galectins reside in the cytosol for a long time (not typical of secreted proteins). From cytosol galectins are targeted to the nucleus, specific cytososlic sites, or are secreted (induced or constitutively) by a non-classical (non-ER-Golgi) pathway, as yet unknown, but possibly similar to the export of interleukin-1, IL-1 (Leffler et al. 2004 Glycoconj. J. 19: 433-440).

Galectins function in all these compartments, for example galectin-3 is implicated in RNA splicing in the nucleus, inhibition of apoptosis in the cytosol, and a variety of extracellular effects on cell signaling and adhesion. Galectin-7 and galectin-12 act in the cytosol by enhancing apoptosis and regulating the cell cycle and differentiation in certain cells (Leffler, H., editor, (2004b) Special Issue on Galectins. Glycoconj. J. 19: 433-638). Most galectins function extracellularly by cross-linking glycoproteins (e.g. laminin, integrins, and IgE receptors) possibly by forming supramolecular ordered arrays (Brewer et al. 2002 Curr. Opin. Struct. Biol. 12: 616-623) and thereby modulating cell adhesion and inducing intracellular signals. Amino acid sequences and homology data for galectin proteins are shown in Panjwani, U.S. patent application number 2010/0004163 A1 published Jan. 7, 2010, which is incorporated by reference herein in its entirety.

Without being limited by any particular theory or mechanism of action, it is here envisioned that galectin proteins are important modulators of angiogenesis, and that an inhibitor of expression and/or activity of a galectin protein inhibits angiogenesis by modulating VEGF signaling by binding to a VEGF receptor.

### Galectin Inhibitor

Herein are provided compositions, methods and kits for treating or preventing ocular angiogenesis or ocular fibrosis by administering a galactoside inhibitor of the expression and/or activity of a galectin protein. In certain embodiments a galactoside inhibitor TD139 is used to modulate activity of galectin-3 protein and to inhibit ocular angiogenesis or ocular fibrosis. Galectin inhibitors used in various compositions, methods and kits herein for inhibiting angiogenesis or fibrosis are found for example in Nilsson et al. U.S. publication number 2004/0147730 A1 (Ser. No. 10/466,933) published Jul. 29, 2004; Nilsson et al. U.S. publication number 2007/0185039 (Ser. No. 11/561,124) published Aug. 7, 2007; Leffler et al. U.S. publication number 2007/0185041 (Ser. No. 11/561,465) published Aug. 9, 2007; Nilsson et al. U.S. publication number 2011/0130553 (Ser. No. 12/992,328) published Jul. 29, 2004; and Leffler et al. U.S. patent publication number 2012/0165277 (Ser. No. 13/266,960) published Jun. 28, 2012, each of which is incorporated by reference herein in its entirety.

Compositions, methods and kits described herein use inhibitors of a galectin protein to selectively inhibit and modulate angiogenesis or fibrosis without negatively affecting other desired processes in the body. Galectin inhibitors are shown in Examples herein to modulate the angiogenic pathway involving VEGF signaling though VEGF receptor-2 in model systems for ocular angiogenesis.

Galectin-3 has been shown to prolong cell surface residence and thus enhance responsiveness of the TGF-β receptor (Partridge et al. 2004 Science 306: 120-124), which regulates alternative macrophage differentiation into M2 macrophages (MacKinnon et al. 2008. J. Immun. 180: 2650-2658). Galectin-3 has also been shown to play a central role in the recruitment and activation of fibroblasts and thus to formation of scar tissue in various organs, including the kidney, liver and lungs (MacKinnon et al Am J Respir Crit Care Med 2012; 185(5):537-46, Henderson et al Am J Pathol 2008:172; 288, Henderson et al Proc Natl Acad Sci 2006: 103(13); 5060). Without being limited by any particular theory or mechanism of action, it is here envisioned that galectin-3 is an endogenous enhancer of TGF-β signaling and alternative macrophage differentiation, and that galectin-3 inhibitors are useful in treating ocular fibrosis and adverse tissue remodeling.

Immunohistochemical studies have shown abnormal expression patterns and levels for galectins in cancer (van den Brule et. al. and Bidon et al. in Leffler, H., editor, (2004b) Special Issue on Galectins. Glycoconj. J. 19: 433-638). Galectin-3 is a histochemical marker of thyroid cancer, and neoexpression of galectin-4 is a promising marker of early breast cancer (Huflejt, M. E. and Leffler, H. 2004 Glycoconj. J. 20: 247-255; and Leffler et al. 2004 Glycoconj. J. 19: 433-440). Evidence for a role of galectin-3 in cancer has been obtained in model systems such as mouse models (Takenaka et al. in Leffler, H., editor, (2004b) Special Issue on Galectins. Glycoconj. J. 19: 433-638). Using paired tumor cell lines (with decreased or increased expression of galectin-3), inducting galectin-3 produced an increase in tumors and metastasis in subjects, and that the suppression of galectin-3 resulted in less tumors and metastasis.

Without being limited by any particular theory or mechanism of action, it is here envisioned that an inhibitor of galectin proteins (e.g., galectin-3) is effective for inhibiting angiogenesis or fibrosis disorders in the eye.

Galectin-1 induced apoptosis in activated T-cells and had a remarkable immunosuppressive effect on autoimmune disease in vivo. The over-expression of galectins in cancers might aid tumor defense against the T-cell response raised by the host (Rubinstein et al. 2004 Cancer Cell 5: 241-251).

Galectin-1 and galectin-3 deficient mice have been generated (Poirier 2002 Biochem. Soc. Symp. 69: 95-103) and these subjects were observed to be healthy and reproduce normally in animal house conditions. Subsequent studies of galectin-3 deficient mice revealed subtle phenotypes in function of neutrophils and macrophages and in bone formation, and in nerve and muscle cell regeneration/differentiation for the galectin-1 deficient mice (Leffler et al. 2004 Glycoconj. J. 19: 433-440; Poirier 2002 Biochem. Soc. Symp. 69: 95-103; and Watt in in Leffler, H., editor, (2004b) Special Issue on Galectins. Glycoconj. J. 19: 433-638). Galectin-7 and galectin-9 deficient mice and are healthy in animal house conditions. Differences in site of expression, specificity and other properties make it unlikely that different galectins can replace each other functionally. The observations in the null mutant mice would indicate that galectins are not essential for basic life supporting functions as can be observed in normal animal house conditions. Instead galectin proteins may be optimizers of normal function and/or essential in stress conditions not found in animal house conditions. The lack of strong effects in null mutant mice may make galectin inhibitors more favorable as potential drugs. Inhibition of the lectin proteins is likely to have fewer unwanted side effects.

Solid phase binding assays and inhibition assays have identified saccharides and glycoconjugates with the ability to bind galectins (reviewed by Leffer et al. 2001 Galectins structure and function—a synopsis (In Mammalian Carbohydrate Recognition Systems Crocker, P., ed., pages 57-83; and Leffler et al. 2004 Glycoconj. J. 19: 433-440). Galectins bind lactose with a dissociation constant (K_{D}) of 0.5-1 mM. K_{D} is the inverse of the association constant, and a lower K_{D} indicates increased binding affinity between molecules. The binding affinity of D-galactose to galectins is generally about 50-fold to 100-fold lower that the binding affinity of lactose to galectins. The binding affinities of N-acetyllactosamine and related disaccharides are variable as these molecules bind a subset of galectins as well as lactose (K_{D} of about 0.5-1 mM), and other galectins about ten-fold less or more than lactose. Small saccharide ligands are effective in binding galectin-3 proteins carrying blood group A-determinants attached to lactose or lacNAc-residues, and were observed to bind about 50-fold greater than the binding affinity for lactose. Galectin-1 shows no preference for these saccharides.

Larger saccharides of the polylactosamine type have been proposed as preferred ligands for galectins such as galectin-3 protein, but not for galectin-1 (Leffler et al. 1986 J. Biol. Chem. 261: 10119-10126). A modified plant pectin polysaccharide has been determined to bind galectin-3 (Pienta et al. 1995 J Natl Cancer Inst. 1995 Mar. 1; 87(5):348-53).

The above-described natural saccharides that have been identified as galectin-3 ligands are not suitable for use as active components in pharmaceutical compositions, because they are susceptible to acidic hydrolysis in the stomach and to enzymatic degradation. In addition, natural saccharides are hydrophilic in nature, and are not readily absorbed from the gastrointestinal tract following oral administration.

### Synthesis of Inhibitors

Saccharides coupled to amino acids with anti-cancer activity are natural compounds in serum, and synthetic analogues have been made (Glinsky et al. 1996 Cancer Res 56: 5319-5324). Saccharides with lactose or galatose coupled to an amino acid inhibit galectins, with about the same potency as the corresponding underivatized sugar. A chemically modified form of citrus pectin (Platt et al. 1992 J. Natl. Cancer. Inst. 84: 438-442) was described as an inhibitor of galectin-3 and as an anti-tumor agent in vivo (Pienta et al., 1995 J. Natl. Cancer Inst. 94:1854-1862).

Natural oligosaccharides, glycoclusters, glycodendrimers, and glycopolymers are too polar and large to be effectively absorbed by the body and in some cases produce immune responses in patients. Furthermore, they are susceptible to acidic hydrolysis in the stomach and to enzymatic hydrolysis.

A thiodigalactoside molecule is synthetic and hydrolytically stable, and is approximately as efficient as N-acetyllactosamine (Leffler et al. 1986 J. Biol. Chem. 261: 10119-10126). A library of pentapeptides was used to obtain low affinity inhibitors of galectin-1 and -3 proteins having similar K_{D} values to that of galactose (Arnusch et al. 2004 Bioorg. Med. Chem. Lett. 14: 1437-1440). Furthermore, peptides are less ideal agents for targeting galectins in vivo, as peptides are susceptible to hydrolysis and are typically polar. N-Acetyllactosamine derivatives carrying aromatic amides or substituted benzyl ethers at C-3′ are highly efficient inhibitors of galectin-3, with IC₅₀ values as low as 4.8 M, which is a 20-fold improvement in inhibition compared to the natural N-acetyllactosamine disaccharide (Sorme P et al. 2002 Chembiochem. 3(2-3):183-189; and Sirme P et al. 2003 Methods Enzymol. 363: 157-169). N-Acetyllactosamine derivatives are less polar overall, due to the presence of the aromatic amido moieties and are thus more suitable as agents for the inhibition of galectins in vivo. Furthermore, C3-triazolyl galactosides have been demonstrated to be as potent inhibitors as the corresponding C3-amides of some galectins. Hence, any properly structured galactose C3-substituent may confer enhanced galectin affinity.

C3-amido- and C3-triazolyl-derivatised compounds are susceptible to hydrolytic degradation in vivo, due to the presence of a glycosidic bond in the galactose and N-acetyllactosamine saccharide moiety and, although they are potent small molecule inhibitors of galectin-3, even further improved affinity and stability is desirable. Accordingly, inhibitors based on 3,3′-diamido- or 3,3′-ditriazolyl-derivatization of thiodigalactoside have been developed, (Cumpstey et al. 2005 Angew. Chem. Int. Ed. 44: 5110-5112; Cumpstey et al. 2008 Chem. Eur. J. 14: 4233-4245; and Dam et al. 2008 Biochemistry 47: 8470-8476; International application numbers WO2005113569 and WO2005113568, U.S. patent publication number 2007/185041, and U.S. Pat. No. 7,638,623 B2, each of which is incorporated by reference herein in its entirety) which lack O-glycosidic hydrolytically and enzymatically labile linkages.

However, 3,3′-derivatized thiodigalactosides have disadvantages including a multistep synthesis involving a double inversion reaction to obtain 3-N-derivatized galactose building blocks. Furthermore, cyclohexane replacement of one galactose ring in thiodigalactoside molecules mimics the galactose ring and provides these galectin-1 and -3 inhibitors with efficiency approaching those of the diamido- and ditriazolyl-thiodigalactoside derivatives (International publication number WO 2010/126435 which is incorporated by reference in its entirety). Replacement of a D-galactopyranose unit with a substituted cyclohexane decreases polarity as well as metabolic susceptibility, thus improving drug-like properties.

Known compounds have the general formulas shown below:

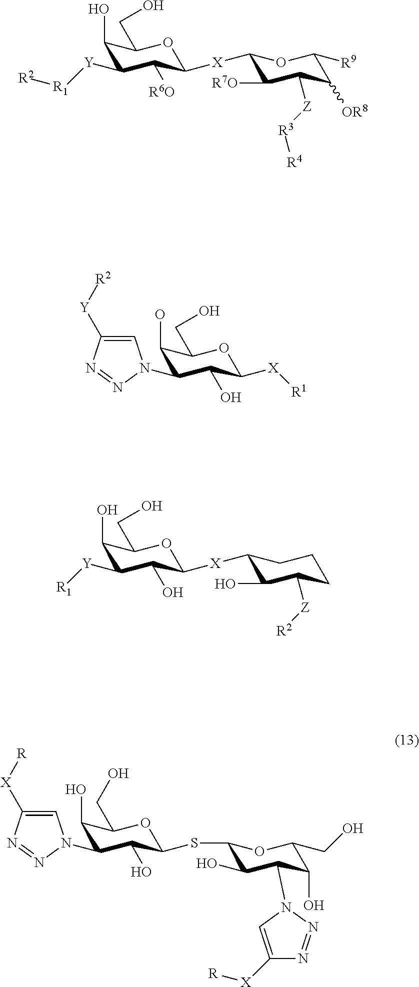

in which in the second structure R¹ can be a D-galactose.

Methods for synthetically preparing galectin protein inhibitors for example for galactosides and intermediates are shown in Nilsson et al. U.S. publication number 2004/0147730 A1 (Ser. No. 10/466,933) published Jul. 29, 2004; Nilsson et al. U.S. publication number 2007/0185039 (Ser. No. 11/561,124) published Aug. 7, 2007; Leffler et al. U.S. publication number 2007/0185041 (Ser. No. 11/561,465) published Aug. 9, 2007; Nilsson et al. U.S. publication number 2011/0130553 (Ser. No. 12/992,328) published Jul. 29, 2004; and Leffler et al. U.S. patent publication number 2012/0165277 (Ser. No. 13/266,960) published Jun. 28, 2012, each of which is incorporated by reference herein in its entirety. Methods for synthesizing the galactosides include reacting a 3-azido-galactosyl thiouronium salt derivative, which is activated to the corresponding thiol in situ, with a 3-azido-galactosyl bromide resulting in the 3,3′-di-azido-thio-di-galactoside.

In certain embodiments, a pharmaceutical composition for inhibiting angiogenesis or fibrosis in the eye includes a bis-(3-deoxy-3-{3-fluorophenyl-1H-1,2,3-triazol-1-yl}-β-D-galactopyranosyl) sulfane, which has a structure shown below:

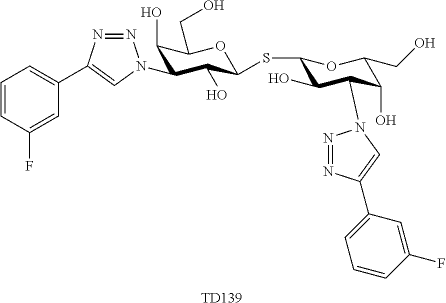

such that the compound inhibits angiogenesis in the eye of the subject. See Mackinnon et al. Am. J. Respir. Crit. Care Med. Mar. 1, 2012 vol. 185 no. 5 537-546, which is incorporated by reference herein in its entirety. In various embodiments, the composition is a beta-galactoside, which is derivatized or functionalized, for example, the composition has the following general formula:

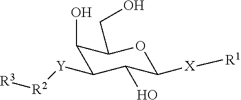

and the configuration of the pyranose ring is D-galacto; X is selected from the group consisting of O, S, NH, CH₂, and NR⁴, or is a bond; Y is selected from the group consisting of NH, CH₂, and NR⁴, or is a bond; R¹ is selected from the group consisting of: a saccharide; hydrogen, an alkyl group, an alkenyl group, an aryl group, a heteroaryl group, and a heterocycle; R² is selected from the group consisting of CO, SO₂, SO, PO, and PO₂; R³ is selected from the group consisting of: an alkyl group of at least 4 carbon atoms, an alkenyl group of at least 4 carbon atoms, an alkyl or alkenyl group of at least 4 carbon atoms substituted with a carboxy group, an alkyl group of at least 4 carbon atoms substituted with both a carboxy group and an amino group, and an alkyl group of at least 4 carbon atoms substituted with a halogen; a phenyl group, a phenyl group substituted with a carboxy group, a phenyl group substituted with at least one halogen, a phenyl group substituted with an alkoxy group, a phenyl group substituted with at least one halogen and at least one carboxy group, a phenyl group substituted with at least one halogen and at least one alkoxy group, a phenyl group substituted with a nitro group, a phenyl group substituted with a sulfo group, a phenyl group substituted with an amine group, a phenyl group substituted with a hydroxy group, a phenyl group substituted with a carbonyl group and a phenyl group substituted with a substituted carbonyl group; or a phenyl amino group; and R⁴ is selected from the group consisting of hydrogen, an alkyl group, an alkenyl group, an aryl group, a heteroaryl group, and a heterocycle. In various embodiments the composition is non-metabolizable, alternatively the composition in various embodiments is metabolizable.

In certain embodiments, the saccharide (R¹) is selected from the group consisting of glucose, mannose, galactose, N-acetylglucosamine, N-acetylgalactosamine, fucose, fructose, xylose, sialic acid, glucuronic acid, iduronic acid, a disaccharide or, an oligosaccharide comprising at least two of the above saccharides, and derivatives thereof. In certain embodiments, Y is NH, X is O, and the halogen is selected from the group consisting of F, Cl, Br and I.

The composition in certain embodiments is selected from the group of: methyl 2-acetamido-2-deoxy-4-O-(3-[3-carboxypropanamido]-3-deoxy-β-D-galactopyranosyl)-β-D-glucopyranoside; methyl 2-acetamido-2-deoxy-4-O-(3-[{Z}-3-carboxypropenamido]-3-deoxy-β-D-galactopyranosyl)-β-D-glucopyranoside; methyl 2-acetamido-2-deoxy-4-O-(3-benzamido-3-deoxy-β-D-galactopyranosyl)-β-D-glucopyranoside; methyl 2-acetamido-2-deoxy-4-O-(3-[2-carboxy-benzamido]-3-deoxy-β-D-galactopyranosyl)-β-D-glucopyranoside; methyl 2-acetamido-2-deoxy-4-O-(3-[4-methoxy-2,3,5,6-tetrafluorbenz-amido]-3-deoxy-β-D-galactopyranosyl)-β-D-glucopyranoside; methyl 2-acetamido-2-deoxy-4-O-(3-[2-carboxy-3,4,5,6-tetrafluorbenzamido]-3-deoxy-β-D-galactopyranosyl)-β-D-glucopyranoside; methyl 2-acetamido-2-deoxy-4-O-(3-methanesulfonamido-3-deoxy-β-D-galactopyranosyl)-β-D-glucopyranoside; methyl 2-acetamido-2-deoxy-4-O-(3-[-4-nitrobenzenesulfonamido]-3-deoxy-β-D-galactopyranosyl)-β-D-glucopyranoside, methyl 2-acetamido-2-deoxy-4-O-(3-phenylaminocarbonylamino-3-deoxy-β-D-galactopyranosyl)-β-D-glucopyranoside; methyl 2-acetamido-2-deoxy-4-O-(3-aminoacetamido-3-deoxy-β-D-galactopyranosyl)-β-D-glucopyranoside; and methyl 2-acetamido-2-deoxy-4-O-(-3-[{2S}-2-amino-3-carboxy-propanamido]-3-deoxy-β-D-galactopyranosyl)-β-D-glucopyranoside.

The composition in various embodiments has the general formula:

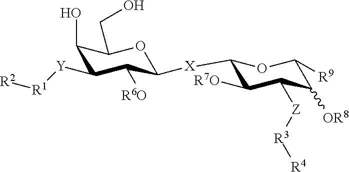

such that the configuration of one of the pyranose rings is β-D-galacto; X is selected from the group consisting of O, S, SO, SO₂, NH, CH₂, and NR⁵, Y is selected from the group consisting of O, S, NH, CH₂, and NR⁵, or is a bond; Z is selected from the group consisting of O, S, NH, CH₂, and NR⁵, or is a bond; R¹ and R³ are independently selected from the group consisting of CO, SO₂, SO, PO₂, PO, and CH₂ or is a bond; and R² and R⁴ are independently selected from the group consisting of: an alkyl group of at least 4 carbons, an alkenyl group of at least 4 carbons, an alkyl group of at least 4 carbons substituted with a carboxy group, an alkenyl group of at least 4 carbons substituted with a carboxy group, an alkyl group of at least 4 carbons substituted with an amino group, an alkenyl group of at least 4 carbons substituted with an amino group, an alkyl group of at least 4 carbons substituted with both an amino and a carboxy group, an alkenyl group of at least 4 carbons substituted with both an amino and a carboxy group, and an alkyl group substituted with one or more halogens; or, a phenyl group substituted with at least one carboxy group, a phenyl group substituted with at least one halogen, a phenyl group substituted with at least one alkoxy group, a phenyl group substituted with at least one nitro group, a phenyl group substituted with at least one sulfo group, a phenyl group substituted with at least one amino group, a phenyl group substituted with at least one alkylamino group, a phenyl group substituted with at least one arylamino group, a phenyl group substituted with at least one dialkylamnino group, a phenyl group substituted with at least one hydroxy group, a phenyl group substituted with at least one carbonyl group and a phenyl group substituted with at least one substituted carbonyl group; or, a naphthyl group, a naphthyl group substituted with at least one carboxy group, a naphthyl group substituted with at least one halogen, a naphthyl group substituted with at least one alkoxy group, a naphthyl group substituted with at least one nitro group, a naphthyl group substituted with at least one sulfo group, a naphthyl group substituted with at least one amino group, a naphthyl group substituted with at least one alkylamino group, a naphthyl group substituted with at least one arylamino group, a naphthyl group substituted with at least one dialkylamnino group, a naphthyl group substituted with at least one hydroxy group, a naphthyl group substituted with at least one carbonyl group and a naphthyl group substituted with at least one substituted carbonyl group; or, a heteroaryl group, a heteroaryl group substituted with at least one carboxy group, a heteroaryl group substituted with at least one halogen, a heteroaryl group substituted with at least one alkoxy group, a heteroaryl group substituted with at least one nitro group, a heteroaryl group substituted with at least one sulfo group, a heteroaryl group substituted with at least one amino group, a heteroaryl group substituted with at least one alkylamino group, a heteroaryl group substituted with at least one dialkylamino group, a heteroaryl group substituted with at least one arylamino group, a heteroaryl group substituted with at least one hydroxy group, a heteroaryl group substituted with at least one carbonyl group and a heteroaryl group substituted with at least one substituted carbonyl group. R⁶ and R⁸ are independently selected from the group consisting of a hydrogen, an acyl group, an alkyl group, a benzyl group, and a saccharide. R⁷ is selected from the group consisting of a hydrogen, an acyl group, an alkyl group, and a benzyl group. R⁹ is selected from the group consisting of a hydrogen, a methyl group. hydroxymethyl group, an acyloxymethyl group, an alkoxymethyl group, and a benzyloxymethyl group.

In certain embodiments, Y is NH, Z is NH, X is S, R¹ is CO, R³ is CO, R² or R⁴ is an aromatic for example an aromatic ring; of R⁶, R⁷, and R⁸ is hydrogen; or R⁹ is a hydroxymethyl group. In certain embodiments, the composition is bis-(3-deoxy-3-benzamido-β-galactopyranosyl)sulfane, bis-(3-deoxy-3-(3-methoxybenzamido)-β-D-galactopyranosyl)sulfane; bis-(3-deoxy-(3,5-dimethoxybenzamido)-β-D-galactopyranosyl)sulfane; bis-(3-deoxy-3-(4-nitrobenzamido)-β-D-galactopyranosyl)sulfane; bis(3-deoxy-3-(2-naphthamido)-β-D-galactopyranosyl)sulfane; bis-(3-deoxy-3-(4-methoxybenzamido)-β-D-galactopyranosyl)sulfane; bis-(3-deoxy-3-(3-nitrobenzamido)-β-D-galactopyranosyl)sulfane; bis-(3-deoxy-3-[4-(dimethylamino)-benzamido]-β-D-galactopyranosyl)sulfane; bis-(3-deoxy-3-(4-methylbenzamido)-β-D-galactopyranosyl)sulfane; bis-(3-deoxy-3-(4-chlorobenzamido)-β-D-galactopyranosyl)sulfane; bis-(3-deoxy-3-(4-tert-butylbenzamido)-β-D-galactopyranosyl)sulfane; bis-(3-deoxy-3-(4-acetylbenzamido)-β-D-galactopyranosyl)sulfane; bis-(3-deoxy-3-[2-(3-carboxy)-naphthamido]-β-D-galactopyranosyl)sulfane; bis-[3-deoxy-3-(3,4-methylenedioxyl)benzamido]-β-D-galactopyranosyl)sulfane, bis-(3-deoxy-3-(4-methoxycarbonylbenzamido)-β-D-galactopyranosyl)sulfane; bis-(3-deoxy-3-(3-carboxybenzamido)-β-D-galactopyranosyl)sulfane; bis-(3-deoxy-3-(3-benzyloxy-5-hydroxy-benzamido)-β-D-galactopyranosyl)sulfane; bis-(3-deoxy-3-(3,5-dibenzyloxybenzamido)-β-D-galactopyranosyl)sulfane; bis-(3-deoxy-3-(3-benzyloxy-5-methoxy-benzamido)-β-D-galactopyranosyl)sulfane; bis-(3-deoxy-3-(3-benzyloxy-5-nonyloxy-benzamido)-β-D-galactopyranosyl)sulfane; bis-(3-deoxy-3-(3-hydroxy-5-methoxy-benzamido)-β-D-galactopyranosyl)-sulfane; bis-(3-deoxy-3-(3-hydroxy-5-nonyloxy-benzamido)-β-D-galactopyranosyl)sulfane, bis-(3-deoxy-3-[3-benzyloxy-5-(4-fluoro-benzyloxy)-benzamido]-β-D-galactopyranosyl)sulfane; bis-(3-deoxy-3-[3-methoxy-5-(4-methyl-benzyloxy)-benzamido]-β-D-galactopyranosyl)sulfane; or bis-(3-deoxy-3-(3-allyloxy-5-benzyloxy-benzamido)-β-D-galactopyranosyl)sulfane.

The composition in various embodiments comprises a 3-triaxolyl-galactoside, for example the composition has a general formula shown below:

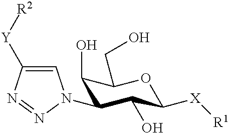

such that the configuration of the pyranose ring is D-galacto; X is selected from the group consisting of O, S, NH, CH₂, and NR⁴, or is a bond; Y is selected from the group consisting of CH₂, CO, SO₂, SO, PO₂ and PO, phenyl, or is a bond; R₁ is selected from the group consisting of: a saccharide; a substituted saccharide; D-galactose; substituted D-galactose; C3-[1,2,3]-triazol-1-yl-substituted D-galactose; a hydrogen, an alkyl group, an alkenyl group, an aryl group, a heteroaryl group, and a heterocycle and derivatives thereof; or an amino group, a substituted amino group, an imino group, or a substituted imino group; and, R² is selected from the group consisting of; hydrogen, an amino group, a substituted amino group, an alkyl group, a substituted alkyl group, an alkenyl group, a substituted alkenyl group, an alkynyl group, a substituted alkynyl group, an alkoxy group, a substituted alkoxy group, an alkylamino group, a substituted alkylamino group, an arylamino group, a substituted arylamino group, an aryloxy group, a substituted aryloxy group, an aryl group, a substituted aryl group, a heteroaryl group, a substituted heteroaryl group, and a heterocycle, a substituted heterocycle.

The saccharide in various embodiments is selected from the group consisting of glucose, mannose, galactose, N-acetylglucosamine, N-acetylgalactosamine, fucose, fructose, xylose, sialic acid, glucuronic acid, iduronic acid, galacturonic acid, a disaccharide or an oligosaccharide comprising at least two of the above saccharides, and derivatives thereof.

In various embodiments of the composition, Y is CO, SO₂, or a bond; R² is an amine or an aryl group; R¹ is galactose, glucose or N-acetylglucosamine; R¹ is substituted galactose, glucose or N-acetylglucosamine; R¹ is a C3-[1,2,3]-triazol-1-yl-substituted galactose; or X is O or S.

In various embodiments of the composition, R2 is a substituted phenyl group wherein said substituent is one or more selected from the group consisting of halogen, alkoxy, alkyl, nitro, sulfo, amino, hydroxy or carbonyl group.

The composition in various embodiments is methyl 3-deoxy-3-(1H-[1,2,3]-triazol-1-yl)-1-thio-β-D-galactopyranoside; methyl 3-deoxy-3-(4-propyl-1H-[1,2,3]-triazol-1-yl)-1-thio-β-D-galactopyranoside; methyl 3-(4-methoxycarbonyl-1H-[1,2,3]-triazol-1-yl)-3-deoxy-1-thio-β-D-galactopyranoside; methyl 3-deoxy-3-(4-(1-hydroxy-1-cyclohexyl)-1H-[1,2,3]-triazol-1-yl)-1-thio-β-D-galactopyranoside; methyl 3-deoxy-3-(4-phenyl-1H-[1,2,3]-triazol-1-yl)-1-thio-β-D-galactopyranoside; methyl 3-deoxy-3-(4-p-tolylsulfonyl-1H-[1,2,3]-triazol-1-yl)-1-thio-β-galactopyranoside; methyl 3-(4-methylaminocarbonyl-1H-[1,2,3]-triazol-1-yl)-3-deoxy-1-thio-β-D-galactopyranoside; methyl 3-(4-butylaminocarbonyl-1H-[1,2,3]-triazol-1-yl)-3-deoxy-1-thio-β-D-galactopyranoside; methyl 3-(4-benzylaminocarbonyl-1H-[1,2,3]-triazol-1-yl)-3-deoxy-1-thio-β-D-galactopyranoside; methyl 3-{4-(3-hydroxyprop-1-ylaminocarbonyl)-1H-[1,2,3]-triazol-1-yl}-3-deoxy-1-thio-β-D-galactopyranoside; methyl 3-{4-[2-(N-morpholino)-ethylaminocarbonyl]-1H-[1,2,3]-triazol-1-yl}-3-deoxy-1-thio-β-D-galactopyranoside; methyl 3-(4-methylaminocarbonyl-1H-[1,2,3]-triazol-1-yl)-3-deoxy-β-D-galactopyranosyl-(1→4)-2-acetamido-2-deoxy-β-D-glucopyranoside, bis-(3-deoxy-3-(4-(methylaminocarbonyl)-1H-[1,2,3]-triazol-1-yl)-β-D-galactopyranosyl)sulfane, methyl 3-deoxy-3-{4-(2-fluorophenyl)-1H-[1,2,3]-triazol-1-yl}-1-thio-β-D-galactopyranoside; methyl 3-deoxy-3-{4-(2-methoxyphenyl)-1H-[1,2,3]-triazol-1-yl}-1-thio-β-D-galactopyranoside; methyl 3-deoxy-3-{4-(3-methoxyphenyl)-1H-[1,2,3]-triazol-1-yl}-1-thio-β-D-galactopyranoside; methyl 3 deoxy-3-{4-(4-methoxyphenyl)-1H-[1,2,3]-triazol-1-yl}-1-thio-β-D-galactopyranoside; methyl 3-deoxy-3-{4-(3,5-dimethoxyphenyl)-1H-[1,2,3]-triazol-1-yl}-1-thio-β-D-galactopyranoside; methyl 3-deoxy-3-{4-(1-naphthyl)-1H-[1,2,3]-triazol-1-yl}-1-thio-β-D-galactopyranoside; methyl 3-deoxy-3-{4-(2-naphthyl)-1H-[1,2,3]-triazol-1-yl}-1-thio-β-D-galactopyranoside; methyl 3-deoxy-3-{4-(2-pyridyl)-1H-[1,2,3]-triazol-1-yl}-1-thio-β-D-galactopyranoside; methyl 3-deoxy-3-{4-(3-pyridyl)-1H-[1,2,3]-triazol-1-yl}-1-thio-β-D-galactopyranoside; methyl 3-deoxy-3-{4-(4-pyridyl)-1H-[1,2,3]-triazol-1-yl}-1-thio-β-D-galactopyranoside; O-{3-deoxy-3-[4-phenyl-[1H-[1,2,3]-triazol-1-yl]-β-D-galactopyranosy-l}-3-indol-carbaldoxim; O-{3-deoxy-3-[4-(methylaminocarbonyl)-1H-[1,2,3]-triazol-1-yl]-β-D-galactopyranosyl}-3-indol-carbaldoxim; O-{3-deoxy-3-[4-phenyl-[1H-[1,2,3]-triazol-1-yl]-β-D-galactopyranosy-l}-(2-hydroxy-5-nitro-phenyl)-carbaldoxim; O-{3-deoxy-3-[4-(methylaminocarbonyl)-1H-[1,2,3]-triazol-1-yl]-β-galactopyranosyl}-(2-hydroxy-5-nitro-phenyl)-carbaldoxim; O-{3-deoxy-3-[4-phenyl-[1H-[1,2,3]-triazol-1-yl]-3-D-galactopyranosy-l}-(2,5-dihydroxyphenyl)-carbaldoxim; O-{3-deoxy-3-[4-(methylaminocarbonyl)-1H-[1,2,3]-triazol-1-yl]-β-galactopyranosyl}-(2,5-dihydroxyphenyl)-carbaldoxim; O-{3-deoxy-3-[4-phenyl-[1H-[1,2,3]-triazol-1-yl]-β-D-galactopyranosy-1}-1-naphthyl-carbaldoxim; or O-{3-deoxy-3-[4-(methylaminocarbonyl)-1H-[1,2,3]-triazol-1-yl]-(3-D-galactopyranosyl}-1-naphthyl-carbaldoxim.

The composition in various embodiments has a thiodigalactoside, for example the composition has a general formula shown below:

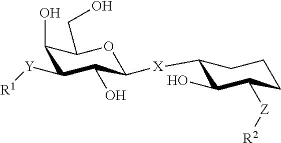

such that the configuration of the pyranose ring is D-galacto; X is selected from the group consisting of O, S, and SO; Y and Z are independently selected from being CONH or a 1H-1,2,3-triazole ring; R¹ and R² are independently selected from the group consisting of: an alkyl group of at least 4 carbons, an alkenyl group of at least 4 carbons, an alkynyl group of at least 4 carbons; a carbamoyl group, a carbamoyl group substituted with an alkyl group, a carbamoyl group substituted with an alkenyl group, a carbamoyl group substituted with an alkynyl group, a carbamoyl group substituted with an aryl group, a carbamoyl group substituted with an substituted alkyl group, and a carbamoyl group substituted with an substituted aryl group; a phenyl group substituted with at least one carboxy group, a phenyl group substituted with at least one halogen, a phenyl group substituted with at least one alkyl group, a phenyl group substituted with at least one alkoxy group, a phenyl group substituted with at least one trifluoromethyl group; a phenyl group substituted with at least one trifluoromethoxy group, a phenyl group substituted with at least one sulfo group, a phenyl group substituted with at least one hydroxy group, a phenyl group substituted with at least one carbonyl group, and a phenyl group substituted with at least one substituted carbonyl group; a naphthyl group, a naphthyl group substituted with at least one carboxy group, a naphthyl group substituted with at least one halogen, a naphthyl group substituted with at least one alkyl group, a naphthyl group substituted with at least one alkoxy group, a naphthyl group substituted with at least one sulfo group, a naphthyl group substituted with at least one hydroxy group, a naphthyl group substituted with at least one carbonyl group, and a naphthyl group substituted with at least one substituted carbonyl group; a heteroaryl group, a heteroaryl group substituted with at least one carboxy group, a heteroaryl group substituted with at least one halogen, a heteroaryl group substituted with at least one alkoxy group, a heteroaryl group substituted with at least one sulfo group, a heteroaryl group substituted with at least one arylamino group, a heteroaryl group substituted with at least one hydroxy group, a heteroaryl group substituted with at least one halogen, a heteroaryl group substituted with at least one carbonyl group, and a heteroaryl group substituted with at least one substituted carbonyl group; and a thienyl group, a thienyl group substituted with at least one carboxy group, a thienyl group substituted with at least one halogen, a thienyl thienyl group substituted with at least one alkoxy group, a thienyl group substituted with at least one sulfo group, a thienyl group substituted with at least one arylamino group, a thienyl group substituted with at least one hydroxy group, a thienyl group substituted with at least one halogen, a thienyl group substituted with at least one carbonyl group, and a thienyl group substituted with at least one substituted carbonyl group.

The composition in various embodiment has a formula in which Y is CONH; the CONH group is linked via the N atom to the pyranose ring; the Z is CONH fore example the CONH group is linked via the N atom to the cyclohexane; or Y is a 1H-1,2,3-triazole ring for example the 1H-1,2,3-triazole ring is linked via the N1 atom to the pyranose ring. In various embodiments of the composition, R¹ is linked to the C4 atom of the 1H-1,2,3-triazole ring; Z is a 1H-1,2,3-triazole ring for example the 1H-1,2,3-triazole ring is linked via the N1 atom to the cyclohexane; or R² is linked to the C4 atom of the 1H-1,2,3-triazole ring.

In various embodiments, the composition includes a digalactoside, for example the composition includes a general formula (13)

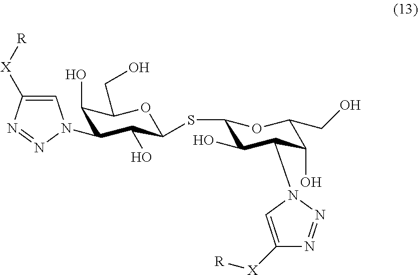

wherein the configuration of at least one of the pyranose rings is D-galacto; X is a bond; R is a phenyl group, which is substituted in any position with one or more substituents selected from the group consisting of methyl, ethyl, isopropyl, tert-butyl, fluoro, chloro, bromo, and trifluoromethyl or R is a thienyl group.

In various embodiments of the composition R is a phenyl group which is substituted in any position with one or more substituents selected from the group consisting of fluoro, chloro, and bromo. For instance R is a phenyl group which is substituted in any position with one or more substituents selected from fluoro. Typically, the configuration of both pyranose rings is D-galacto.

The term “alkyl group” as used herein includes chemical compounds that consists only of hydrogen and carbon atoms that are bonded by single bonds. For example an alkyl group comprises from about one carbon atom to about seven carbon atoms, and in various embodiments includes about one carbon atom to about four carbon atoms. The alkyl group may be straight- or branched-chain and may also form a cycle comprising from three to seven carbon atoms, such as three, four, five, six, or seven carbon atoms. Thus alkyl refers to any of methyl, ethyl, n-propyl, isopropyl, n-butyl, sec-butyl, tert-butyl, pentyl, isopentyl, 3-methylbutyl, 2,2-dimethylpropyl, n-hexyl, 2-methylpentyl, 2,2-dimethylbutyl, 2,3-dimethylbutyl, n-heptyl, 2-methylhexyl, 2,2-dimethylpentyl, 2,3-dimethylpentyl, cyclopropyl, cyclobutyl, cyclopentyl, cyclohexyl, cycloheptyl, and 1-methylcyclopropyl.

The term “alkenyl group” as used herein is any functional group or substituent comprising at least one double bond. The alkenyl group includes from about two carbon atoms to about seven carbon atoms. The alkenyl group includes any of vinyl, allyl, but-1-enyl, but-2-enyl, 2,2-dimethylethenyl, 2,2-dimethylprop-1-enyl, pent-1-enyl, pent-2-enyl, 2,3-dimethylbut-1-enyl, hex-1-enyl, hex-2-enyl, hex-3-enyl, prop-1,2-dienyl, 4-methylhex-1-enyl, cycloprop-1-enyl group, and others.

The term “alkylated” as used herein means substituted with an alkyl group. The term “alkenylated” as used herein refers to being substituted with an alkenyl group,

The term “aryl group” as used herein refers to any functional group or substituent derived from an aromatic ring and having about four carbon atoms to about twelve carbon atoms. The aryl group may for example be a phenyl group or a naphthyl group. The above-mentioned groups may be substituted with any other known substituents within the art of organic chemistry. The groups may also be substituted with two or more of the the substituents. Examples of substituents are halogen, alkyl, alkenyl, alkoxy, nitro, sulfo, amino, hydroxy, and carbonyl groups. Halogen substituents are bromo, fluoro, iodo, and chloro. Alkyl groups for example include about one carbon atom to about seven carbon atoms. Alkenyl groups include for example two to seven carbon atoms, such as two carbon atoms or four carbon atoms. Alkoxy groups include one carbon atom to seven carbon atoms, which may contain an unsaturated carbon atom. Combinations of substituents can be present such as trifluoromethyl.

The term “alkoxy group” as used herein is a functional group or substituent including carbon atoms bonded to an oxygen, for example about one carbon atom to about seven carbon atoms. The alkoxy group may be a methoxy group, an ethoxy group, a propoxy group, a isopropoxy group, a n-butoxy group, a sec-butoxy group, tert-butoxy group, pentoxy group, isopentoxy group, 3-methylbutoxy group, 2,2-dimethylpropoxy group, n-hexoxy group, 2-methylpentoxy group, 2,2-dimethylbutoxy group 2,3-dimethylbutoxy group, n-heptoxy group, 2-methylhexoxy group, 2,2-dimethylpentoxy group, 2,3-dimethylpentoxy group, cyclopropoxy group, cyclobutoxy group, cyclopentyloxy group, cyclohexyloxy group, cycloheptyloxy group, and 1-methylcyclopropyl oxy group.

The term “alkylamino group” as used herein is a functional group or substituent including an alkyl group (about one carbon atom to about seven carbon atoms) bond to a nitrogen atom with a lone pair. For example the alkyl group is any of methyl, ethyl, n-propyl, isopropyl, n-butyl, sec-butyl, tert-butyl, pentyl, isopentyl, 3-methylbutyl, 2,2-dimethylpropyl, n-hexyl, 2-methylpentyl, 2,2-dimethylbutyl, 2,3-dimethylbutyl, n-heptyl, 2-methylhexyl, 2,2-dimethylpentyl, 2,3-dimethylpentyl, cyclopropyl, cyclobutyl, cyclopentyl, cyclohexyl, cycloheptyl, and 1-methylcyclopropyl.

The term “arylamino group” as used herein is an aryl group that is bonded to a derivative of ammonia, such that the derivative of ammonia contains a basic nitrogen atom with a lone pair of electrons. The arylamino group has for example an aryl group having about four carbon atoms to about seven carbon atoms. The arylamino group for example is aniline, carboxylated aniline or halogenated aniline, halogen being as defined above.

The term “aryloxy group” as used herein is an aryl functional group bound to an oxygen. For example the aryloxy group includes about four carbon atoms to about twelve carbon atoms. The aryloxy group may be phenol, carboxylated phenol or halogenated phenol, such that halogen is as defined above.

The term “heteroaryl group” as used herein in an aryl group comprising from about four carbon atoms to about 18 carbon atoms, such that at least one atom of the ring is a heteroatom, i.e. not a carbon. In various embodiments, the heteroatom is N, O or S. The heteroaryl group in certain embodiments is a pyridine, or an indole group.

The above-mentioned groups may be substituted with any other known substituents within the art of organic chemistry. The groups may also be substituted with two or more of the substituents. Examples of substituents are halogen, alkoxy, nitro, sulfo, amino, hydroxy, and carbonyl groups. Halogen substituents are bromo, fluoro, iodo, and chloro. In various embodiments, the alkyl groups contain about one carbon atom to about seven carbon atoms. The alkenyl groups in various embodiments include about to two carbon atoms to about seven carbon atoms. The alkoxy in various embodiments includes about one carbon atom to about seven carbon atoms, preferably one to four carbon atoms, and may contain an unsaturated carbon atom.

The term “subject” as used herein refers in various embodiments to mammals and includes humans, primates, livestock animals (e.g., sheep, pigs, cattle, horses, and donkeys), laboratory test animals (e.g., mice, rabbits, rats, and guinea pigs), companion animals (e.g., dogs and cats) and high value zoo and captive wild animals (e.g., foxes, kangaroos, elephants, and deer).

### Polynucleotide Inhibitors

Embodiments of the invention herein, provide a method for inhibiting or preventing ocular angiogenesis or ocular fibrosis, or an ocular angiogenesis-associated disorder or an ocular fibrosis-associated disorder, the method including contacting cells or tissue with a pharmaceutical composition including an inhibitor or modulator of a galectin protein or a source of modulator expression. For example, the inhibitor is a recombinantly produced protein administered in situ or ex vivo. The term “recombinant” refers to proteins produced by manipulation of genetically modified organisms, for example micro-organisms or eukaryotic cells in culture.

In an embodiment of the invention, the compositions, methods and kits include a source of the modulator which is an inhibitor such as that a polynucleotide sequences that encode the inhibitory protein, for example the polynucleotide sequence is engineered into recombinant DNA molecules to direct expression of the inhibitory protein or a portion thereof in appropriate host cells. To express a biologically active inhibitor, a nucleotide sequence encoding the inhibitor, or functional equivalent, is inserted into an appropriate expression vector, i.e., a vector that contains the necessary nucleic acid encoding elements that regulate transcription and translation of the inserted coding sequence, operably linked to the nucleotide sequence encoding the amino acid sequence of the inhibitory protein.

Methods that are well known to those skilled in the art are used to construct expression vectors containing a nucleic acid sequence encoding for example a protein or a peptide operably linked to appropriate transcriptional and translational control elements. These methods include in vitro recombinant DNA techniques, synthetic techniques and in vivo recombination or genetic recombination. Techniques are described in Sambrook et al., Molecular Cloning: A Laboratory Manual, Cold Spring Harbor Press, Plainview, N.Y., 1989.

A variety of commercially available expression vector/host systems are useful to contain and express a sequence that encodes a protein or a peptide. These include but are not limited to microorganisms such as bacteria transformed with recombinant bacteriophage, plasmid or cosmid DNA expression vectors; yeast transformed with yeast expression vectors; insect cell systems contacted with virus expression vectors (e.g., baculovirus); plant cell systems transfected with virus expression vectors (e.g., cauliflower mosaic virus, CaMV; tobacco mosaic virus, TMV) or transformed with bacterial expression vectors (e.g., Ti, pBR322, or pET25b plasmid); or animal cell systems. See Ausubel et al., Current Protocols in Molecular Biology, John Wiley & Sons, New York, N.Y., 1989.

Virus vectors include, but are not limited to, adenovirus vectors, lentivirus vectors, retrovirus vectors, adeno-associated virus (AAV) vectors, and helper-dependent adenovirus vectors. For example, the vectors deliver a nucleic acid sequence that encodes a transcription factor or agent that binds to a transcription that as shown herein modulates trans-differentation of muscle satellite cells. Adenovirus packaging vectors are commercially available from American Type Tissue Culture Collection (Manassas, Va.). Methods of constructing adenovirus vectors and using adenovirus vectors are shown in Klein et al., Ophthalmology, 114:253-262, 2007 and van Leeuwen et al., Eur. J. Epidemiol., 18:845-854, 2003.

Adenovirus vectors have been used in eukaryotic gene expression (Levrero et al., Gene, 101:195-202, 1991) and vaccine development (Graham et al., Methods in Molecular Biology: Gene Transfer and Expression Protocols 7, (Murray, Ed.), Humana Press, Clifton, N.J., 109-128, 1991). Further, recombinant adenovirus vectors are used for gene therapy (Wu et al., U.S. Pat. No. 7,235,391 issued Jun. 26, 2007).

Recombinant adenovirus vectors are generated, for example, from homologous recombination between a shuttle vector and a provirus vector (Wu et al., U.S. Pat. No. 7,235,391). Helper cell lines for use in these recombinant adenovirus vectors may be derived from human cells such as, 293 human embryonic kidney cells, muscle cells, hematopoietic cells or other human embryonic mesenchymal or epithelial cells. Alternatively, the helper cells may be derived from the cells of other mammalian species that are permissive for human adenovirus, e.g., Vero cells or other monkey embryonic mesenchymal or epithelial cells. Generation and propagation of these replication defective adenovirus vectors using a helper cell line is described in Graham et al, 1997 J. Gen. Virol., 36:59-72, 1977.

Lentiviral vector packaging vectors are commercially available from Invitrogen Corporation (Carlsbad Calif.). An HIV-based packaging system for the production of lentiviral vectors is prepared using constructs in Naldini et al., Science 272: 263-267, 1996; Zufferey et al., Nature Biotechnol., 15: 871-875, 1997; and Dull et al., J. Virol. 72: 8463-8471, 1998.

A number of vector constructs are available to be packaged using a system, based on third-generation lentiviral SIN vector backbone (Dull et al., J. Virol. 72: 8463-8471, 1998). For example the vector construct pRRLsinCMVGFPpre contains a 5′ LTR in which the HIV promoter sequence has been replaced with that of Rous sarcoma virus (RSV), a self-inactivating 3′ LTR containing a deletion in the U3 promoter region, the HIV packaging signal, RRE sequences linked to a marker gene cassette consisting of the Aequora jellyfish green fluorescent protein (GFP) driven by the CMV promoter, and the woodchuck hepatitis virus PRE element, which appears to enhance nuclear export. The GFP marker gene allows quantitation of transfection or transduction efficiency by direct observation of UV fluorescence microscopy or flow cytometry (Kafri et al., Nature Genet., 17: 314-317, 1997 and Sakoda et al., J. Mol. Cell. Cardiol., 31: 2037-2047, 1999).

Manipulation of retroviral nucleic acids to construct a retroviral vector containing a gene that encodes a protein, and methods for packaging in cells are accomplished using techniques known in the art. See Ausubel, et al., 1992, Volume 1, Section III (units 9.10.1-9.14.3); Sambrook, et al., 1989. Molecular Cloning: A Laboratory Manual. Second Edition. Cold Spring Harbor Laboratory Press, Cold Spring Harbor, N.Y.; Miller, et al., Biotechniques. 7:981-990, 1989; Eglitis, et al., Biotechniques. 6:608-614, 1988; U.S. Pat. Nos. 4,650,764, 4,861,719, 4,980,289, 5,122,767, and 5,124,263; and PCT patent publications numbers WO 85/05629, WO 89/07150, WO 90/02797, WO 90/02806, WO 90/13641, WO 92/05266, WO 92/07943, WO 92/14829, and WO 93/14188.

A retroviral vector is constructed and packaged into non-infectious transducing viral particles (virions) using an amphotropic packaging system. Examples of such packaging systems are found in, for example, Miller, et al., Mol. Cell Biol. 6:2895-2902, 1986; Markowitz, et al., J. Virol. 62:1120-1124, 1988; Cosset, et al., J. Virol. 64:1070-1078, 1990; U.S. Pat. Nos. 4,650,764, 4,861,719, 4,980,289, 5,122,767, and 5,124,263, and PCT patent publications numbers WO 85/05629, WO 89/07150, WO 90/02797, WO 90/02806, WO 90/13641, WO 92/05266, WO 92/07943, WO 92/14829, and WO 93/14188.

Generation of “producer cells” is accomplished by introducing retroviral vectors into the packaging cells. Examples of such retroviral vectors are found in, for example, Korman, et al., Proc. Natl. Acad. Sci. USA. 84:2150-2154, 1987; Morgenstern, et al., Nucleic Acids Res. 18:3587-3596, 1990; U.S. Pat. Nos. 4,405,712, 4,980,289, and 5,112,767; and PCT patent publications numbers WO 85/05629, WO 90/02797, and WO 92/07943. Herpesvirus packaging vectors are commercially available from Invitrogen Corporation, (Carlsbad, Calif.). Exemplary herpesviruses are an α-herpesvirus, such as Varicella-Zoster virus or pseudorabies virus; a herpes simplex virus such as HSV-1 or HSV-2; or a herpesvirus such as Epstein-Barr virus. A method for preparing empty herpesvirus particles that can be packaged with a desired nucleotide segment is shown in Fraefel et al., U.S. Pat. No. 5,998,208, issued Dec. 7, 1999.

The herpesvirus DNA vector can be constructed using techniques familiar to the skilled artisan. For example, DNA segments encoding the entire genome of a herpesvirus is divided among a number of vectors capable of carrying large DNA segments, e.g., cosmids (Evans, et al., Gene 79, 9-20, 1989), yeast artificial chromosomes (YACS) (Sambrook, J. et al., Molecular Cloning: A Laboratory Manual, 2nd Edition, Cold Spring Harbor Press, Cold Spring Harbor, N.Y., 1989) or E. coli F element plasmids (O'Conner, et al., Science 244:1307-1313, 1989).

For example, sets of cosmids have been isolated which contain overlapping clones that represent the entire genomes of a variety of herpesviruses including Epstein-Barr virus, Varicella-Zoster virus, pseudorabies virus and HSV-1. See M. van Zijl et al., J. Virol. 62, 2191, 1988; Cohen, et al., Proc. Nat'l Acad. Sci. U.S.A. 90, 7376, 1993; Tomkinson, et al., J. Virol. 67, 7298, 1993; and Cunningham et al., Virology 197, 116, 1993.

AAV is a dependent parvovirus in that it depends on co-infection with another virus (either adenovirus or a member of the herpes virus family) to undergo a productive infection in cultured cells (Muzyczka, Curr Top Microbiol Immunol, 158:97 129, 1992). For example, recombinant AAV (rAAV) virus is made by co-transfecting a plasmid containing the gene of interest, for example, the Nkx3.2 gene. Cells are also contacted or transfected with adenovirus or plasmids carrying the adenovirus genes required for AAV helper function. Recombinant AAV virus stocks made in such fashion include with adenovirus which must be physically separated from the recombinant AAV particles (for example, by cesium chloride density centrifugation).

Adeno-associated virus (AAV) packaging vectors are commercially available from GeneDetect (Auckland, New Zealand). AAV has been shown to have a high frequency of integration and infects nondividing cells, thus making it useful for delivery of genes into mammalian cells in tissue culture (Muzyczka, Curr Top Microbiol Immunol, 158:97 129, 1992). AAV has a broad host range for infectivity (Tratschin et al., Mol. Cell. Biol., 4:2072 2081, 1984; Laughlin et al., J. Virol., 60(2):515 524, 1986; Lebkowski et al., Mol. Cell. Biol., 8(10):3988 3996, 1988; McLaughlin et al., J. Virol., 62(6):1963 1973, 1988).

Methods of constructing and using AAV vectors are described, for example in U.S. Pat. Nos. 5,139,941 and 4,797,368. Use of AAV in gene delivery is further described in LaFace et al., Virology, 162(2):483 486, 1988; Zhou et al., Exp. Hematol, 21:928 933, 1993; Flotte et al., Am. J. Respir. Cell Mol. Biol., 7(3):349 356, 1992; and Walsh et al., J. Clin. Invest, 94:1440 1448, 1994.

Recombinant AAV vectors have been used for in vitro and in vivo transduction of marker genes (Kaplitt et al., Nat Genet., 8(2):148 54, 1994; Lebkowski et al., Mol. Cell. Biol., 8(10):3988 3996, 1988; Samulski et al., EMBO J., 10:3941 3950, 1991; Shelling and Smith, Gene Therapy, 1: 165 169, 1994; Yoder et al., Blood, 82 (Supp.): 1:347A, 1994; Zhou et al., Exp. Hematol, 21:928 933, 1993; Tratschin et al., Mol. Cell. Biol., 5:3258 3260, 1985; McLaughlin et al., J. Virol., 62(6):1963 1973, 1988) and transduction of genes involved in human diseases (Flotte et al., Am. J. Respir. Cell Mol. Biol., 7(3):349 356, 1992; Ohi et al., Gene, 89(2):279 282, 1990; Walsh et al., J. Clin. Invest, 94:1440 1448, 1994; and Wei et al., Gene Therapy, 1:261 268, 1994).

### Antibody Inhibitors

The present invention in various embodiments includes an inhibitor of a galectin protein that modulates angiogenesis (e.g., ocular angiogenesis). An embodiment of an galectin inhibitor which is a protein includes an antibody that binds to the galectin protein or to a molecule that affects the expression or activity of the galectin protein. The term “antibody” as referred to herein includes whole antibodies and antigen binding fragments (i.e., “antigen-binding portion”) or single chains of these. A naturally occurring “antibody” is a glycoprotein including at least two heavy (H) chains and two light (L) chains inter-connected by disulfide bonds.

In various embodiments, an antibody that “specifically binds to a galectin protein” refers to an antibody that binds to a galectin protein with a K_{D} sufficient to inhibit or modulate angiogenesis, for example the K_{D} is 5×10⁻⁹ M or less, 2×10⁻⁹ M or less, or 1×10⁻¹⁰ M or less. For example, the antibody is a monoclonal antibody or a polyclonal antibody. The terms “monoclonal antibody” or “monoclonal antibody composition” as used herein refer to a preparation of antibody molecules of single molecular composition. A monoclonal antibody composition displays a single binding specificity and affinity for a transcription factor or for a particular epitope of a transcription factor. The antibody includes for example an IgM, IgE, IgG such as IgG1 or IgG4.

The terms “polyclonal antibody” or “polyclonal antibody composition” refer to a large set of antibodies each of which is specific for one of the many differing epitopes found in the immunogen, and each of which is characterized by a specific affinity for that epitope. An epitope is the smallest determinant of antigenicity, which for a protein, comprises a peptide of six to eight residues in length (Berzofsky, J. and I. Berkower, (1993) in Paul, W., Ed., Fundamental Immunology, Raven Press, N.Y., p. 246). Affinities range from low, e.g. 10⁻⁶ M to high, e.g., 10⁻¹¹ M. The polyclonal antibody fraction collected from mammalian serum is isolated by well known techniques, e.g. by chromatography with an affinity matrix that selectively binds immunoglobulin molecules such as protein A, to obtain the IgG fraction. To enhance the purity and specificity of the antibody, the specific antibodies may be further purified by immunoaffinity chromatography using solid phase-affixed immunogen. The antibody is contacted with the solid phase-affixed immunogen for a period of time sufficient for the immunogen to immunoreact with the antibody molecules to form a solid phase-affixed immunocomplex. Bound antibodies are eluted from the solid phase by standard techniques, such as by use of buffers of decreasing pH or increasing ionic strength, the eluted fractions are assayed, and those containing the specific antibodies are combined.

Also useful for the methods herein is an antibody that is a recombinant antibody. The term “recombinant human antibody”, as used herein, includes antibodies prepared, expressed, created or isolated by recombinant means. Mammalian host cells for expressing the recombinant antibodies used in the methods herein include Chinese Hamster Ovary (CHO cells) including dhfr-CHO cells, described Urlaub and Chasin, Proc. Natl. Acad. Sci. USA 77:4216-4220, 1980 used with a DH FR selectable marker, e.g., as described in R. J. Kaufman and P. A. Sharp, 1982 Mol. Biol. 159:601-621, NSO myeloma cells, COS cells and SP2 cells. In particular, for use with NSO myeloma cells, another expression system is the GS gene expression system shown in WO 87/04462, WO 89/01036 and EP 338,841. To produce antibodies, expression vectors encoding antibody genes are introduced into mammalian host cells, and the host cells are cultured for a period of time sufficient to allow for expression of the antibody in the host cells or secretion of the antibody into the culture medium in which the host cells are grown. Antibodies are recovered from the culture medium using standard protein purification methods.

Standard assays to evaluate the binding ability of the antibodies toward the target of various species are known in the art, including for example, an ELISAs, an western blots and an radio immunoassay (RIA). The binding kinetics (e.g., binding affinity) of the antibodies also can be assessed by standard assays known in the art, such as by Biacore analysis.

General methodologies for antibody production, including criteria to be considered when choosing an animal for the production of antisera, are described in Harlow et al. (Antibodies, Cold Spring Harbor Laboratory, pp. 93-117, 1988). For example, an animal of suitable size such as a goat, a dog, a sheep, a mouse, or a camel is immunized by administration of an amount of immunogen, such as the intact protein or a portion thereof containing an epitope from a human transcription factor, effective to produce an immune response. An exemplary protocol involves subcutaneous injection with 100 micrograms to 100 milligrams of antigen, depending on the size of the animal, followed three weeks later with an intraperitoneal injection of 100 micrograms to 100 milligrams of immunogen with adjuvant depending on the size of the animal, for example Freund's complete adjuvant. Additional intraperitoneal injections every two weeks with adjuvant, for example Freund's incomplete adjuvant, are administered until a suitable titer of antibody in the animal's blood is achieved. Exemplary titers include a titer of at least about 1:5000 or a titer of 1:100,000 or more, i.e., the greatest dilution indicating that having a detectable antibody activity. The antibodies are purified, for example, by affinity purification using binding to columns containing human MAC.

Monoclonal antibodies are generated by in vitro immunization of human lymphocytes. Techniques for in vitro immunization of human lymphocytes are described in Inai, et al., Histochemistry, 99(5):335 362, May 1993; Mulder, et al., Hum. Immunol., 36(3):186 192, 1993; Harada, et al., J. Oral Pathol. Med., 22(4):145 152, 1993; Stauber, et al., J. Immunol. Methods, 161(2):157 168, 1993; and Venkateswaran, et al., Hybridoma, 11(6) 729 739, 1992. These techniques can be used to produce antigen-reactive monoclonal antibodies, including antigen-specific IgG, and IgM monoclonal antibodies. Any antibody or a fragment thereof having affinity and specific for a transcription factor is within the scope of the modulator compositions provided herein.

The term “antibody” as referred to herein includes whole antibodies and any antigen binding fragment (i. e., “antigen-binding portion”) or single chains thereof, for example, Fv fragments. A naturally occurring “antibody” is a glycoprotein comprising at least two heavy (H) chains and two light (L) chains inter-connected by disulfide bonds. Each heavy chain is comprised of a heavy chain variable region (abbreviated herein as V_{H}) and a heavy chain constant region. The heavy chain constant region is comprised of three domains, CH1, CH2 and CH3. Each light chain is comprised of a light chain variable region (abbreviated herein as V_{L}) and a light chain constant region. The light chain constant region is comprised of one domain, C_{L}. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system.

The term “antigen-binding portion” of an antibody (or simply “antigen portion”), as used herein, refers to full length or one or more fragments of an antibody that retain the ability to specifically bind to a target (e.g., to a galectin, or a fragment of a galectin, or to a galectin inhibitor, or to a ligand of a galectin in a tissue). It has been shown that the antigen-binding function of an antibody can be performed by fragments of a full-length antibody. Examples of binding fragments encompassed within the term “antigen-binding portion” of an antibody include a Fab fragment, a monovalent fragment consisting of the V_{L}, V_{H}, C_{L} and CH1 domains; a F(ab)₂ fragment, a bivalent fragment comprising two Fab fragments linked by a disulfide bridge at the hinge region; a Fd fragment consisting of the V_{H} and CH1 domains; a Fv fragment consisting of the V_{L} and V_{H} domains of a single arm of an antibody; a dAb fragment (Ward et al. 1989 Nature 341:544), which consists of a V_{H} domain; and an isolated complementarity determining region (CDR).

Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv); see e.g., Bird R. E. et al. 1988 Science 242:423; and Huston, J. S. et al. 1988 Proc Natl Acad Sci USA 85:5879). Such single chain antibodies are also intended to be encompassed within the term “antigen-binding portion” of an antibody. These antibody fragments are obtained using conventional techniques known to those of skill in the art, and the fragments are screened for utility in the same manner as are intact antibodies.

An “isolated antibody”, as used herein, refers to an antibody that is substantially free of other antibodies having different antigenic specificities (e.g., an isolated antibody that specifically binds a target such as a galectin, or a fragment of a galectin, or to a galectin inhibitor, or to a ligand of a galectin in a tissue, is substantially free of antibodies that specifically bind antigens other than this target). An isolated antibody that specifically binds DEC205 may, however, have cross-reactivity to other antigens, such as corresponding target molecules from other species. Moreover, an isolated antibody may be substantially free of other cellular material and/or chemicals.

The terms “monoclonal antibody” or “monoclonal antibody composition” as used herein refer to a preparation of antibody molecules of single molecular composition. A monoclonal antibody composition displays a single binding specificity and affinity for a particular epitope.

The term “human antibody”, as used herein, is intended to include antibodies having variable regions in which both the framework and CDR regions are derived from sequences of human origin. Furthermore, if the antibody contains a constant region, the constant region also is derived from such human sequences, e.g., human germline sequences, or mutated versions of human germline sequences. The human antibodies of the invention may include amino acid residues not encoded by human sequences (e.g., mutations introduced by random or site-specific mutagenesis in vitro or by somatic mutation in vivo). However, the term “human antibody”, as used herein, is not intended to include antibodies in which CDR sequences derived from the germline of another mammalian species, such as a mouse, have been grafted onto human framework sequences.

The term “human monoclonal antibody” refers to antibodies displaying a single binding specificity which have variable regions in which both the framework and CDR regions are derived from human sequences. In one embodiment, the human monoclonal antibodies are produced by a hybridoma which includes a B cell obtained from a transgenic nonhuman animal, e.g., a transgenic mouse, having a genome comprising a human heavy chain transgene and a light chain transgene fused to an immortalized cell.

The term “recombinant human antibody”, as used herein, includes all human antibodies that are prepared, expressed, created or isolated by recombinant means, such as antibodies isolated from an animal (e.g., a mouse) that is transgenic or transchromosomal for human immunoglobulin genes or a hybridoma prepared therefrom, antibodies isolated from a host cell transformed to express the human antibody, e.g., from a transfectoma, antibodies isolated from a recombinant, combinatorial human antibody library, and antibodies prepared, expressed, created or isolated by any other means that involve splicing of all or a portion of a human immunoglobulin gene, sequences to other DNA sequences. Such recombinant human antibodies have variable regions in which the framework and CDR regions are derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V₁ and V_{L} sequences, may not naturally exist within the human antibody germline repertoire in vivo.

As used herein, “isotype” refers to the antibody class (e.g., IgM, IgE, IgG such as IgG1 or IgG4) that is provided by the heavy chain constant region genes.

The phrases “an antibody recognizing an antigen” and “an antibody specific for an antigen” are used interchangeably herein with the term “an antibody which binds specifically to an antigen.”

As used herein, an antibody or an antibody-fusion protein that specifically binds to a dendritic cell receptor, e.g., to a target which is specifically a human target such as a ligand of a galectin in a human tissue, is intended to refer to an antibody that binds to the human target with a K_{D} of about 5×10⁻⁹ M or less, about 2×10⁻⁹ M or less, or about 1×10⁻¹ M or less. An antibody that “cross-reacts with an antigen other than human target” is intended to refer to an antibody that binds that antigen with a K_{D} of about 0.5×10⁻⁸ M or less, about 5×10⁻⁹ M or less, or about 2×10⁻⁹ M or less. An antibody that “does not cross-react with a particular antigen” is intended to refer to an antibody that binds to that antigen, with a K_{D} of about 1.5×10⁻⁸ M or greater, or a K_{D} of about 5-10×10⁻⁸ M or about 1×10⁻⁷ M or greater.

In certain embodiments, such antibodies that do not cross-react with the antigen exhibit essentially undetectable binding against these proteins in standard binding assays.

As used herein, an antibody that inhibits binding of a target to the galectin refers to an antibody that inhibits a target binding to the receptor with a K of about 1 nM or less, about 0.75 nM or less, about 0.5 nM or less, or about 0.25 nM or less. GL 117 is a bacterial anti-β-galactosidase nonspecific isotype-matched rat monoclonal antibody negative control (Hawiger, D. et al. 2001 J Exp Med 194: 769-779).

The term “K_{assoc}” or “Kₐ”, as used herein, is intended to refer to the association rate of a particular antibody-antigen interaction, whereas the term “K_{dis}” or “K_{D},” as used herein, is intended to refer to the dissociation rate of a particular antibody-antigen interaction. The term “K_{D}”, as used herein, is intended to refer to the dissociation constant, which is obtained from the ratio of K_{d} to Kₐ (i.e. K_{d}/Kₐ) and is expressed as a molar concentration (M). K_{D} values for antibodies can be determined using methods well established in the art. A method for determining the K_{D} of an antibody is by using surface plasmon resonance, or using a biosensor system such as a Biacore® system.

As used herein, the term “affinity” refers to the strength of interaction between antibody and antigen at single antigenic sites. Within each antigenic site, the variable region of the antibody “arm” interacts through weak non-covalent forces with antigen at numerous sites; the more interactions, the stronger the affinity.

As used herein, the term “avidity” refers to an informative measure of the overall stability or strength of the antibody-antigen complex. It is controlled by three major factors: antibody epitope affinity; the valence of both the antigen and antibody; and the structural arrangement of the interacting parts. Ultimately these factors define the specificity of the antibody, that is, the likelihood that the particular antibody is binding to a precise antigen epitope.

As used herein, the term “cross-reactivity” refers to an antibody or population of antibodies binding to epitopes on other antigens. This can be caused either by low avidity or specificity of the antibody or by multiple distinct antigens having identical or very similar epitopes. Cross reactivity is sometimes desirable when one wants general binding to a related group of antigens or when attempting cross-species labeling when the antigen epitope sequence is not highly conserved in evolution.

As used herein, the term “high affinity” for an IgG antibody refers to an antibody having a K_{D} of 10⁻⁸ M or less, 10⁻⁹ M or less, or 10⁻¹⁰ M or less for a target antigen. However, “high affinity” binding can vary for other antibody isotypes. For example, “high affinity” binding for an IgM isotype refers to an antibody having a K_{D} of 10⁻⁷ M or less, or 10⁻⁸ M or less.

Standard assays to evaluate the binding ability of the antibodies toward a target of any of various species are known in the art, including for example, ELISAs, western blots and RIAs. Suitable assays are described in detail in the Examples. The binding kinetics (e.g., binding affinity) of the antibodies also can be assessed by standard assays known in the art, such as by Biacore analysis. Assays to evaluate the effects of the antibodies on functional properties of the antibody (e.g., receptor binding, preventing or ameliorating autoimmune disease) are described in further detail in the Examples.

Accordingly, an antibody that “inhibits” one or more of these galectin-related functional properties (e.g., biochemical, immunochemical, cellular, physiological or other biological activities, or the like) as determined according to methodologies known to the art and described herein, will be understood to relate to a statistically significant decrease in the particular activity relative to that seen in the absence of the antibody (e.g., or when a control antibody of irrelevant specificity is present). An antibody that inhibits a galectin-related activity effects such a statistically significant decrease by at least 10% of the measured parameter, by at least 50%, 80% or 90%, and in certain embodiments an antibody of the invention may inhibit greater than 95%, 98% or 99% of galectin functional activity.

### RNA Interference

Inhibitory agents include RNA interference agents that bind to a nucleic acid that encodes a galectin protein or that encodes a molecule that modulates activity of the galectin protein, such that the nucleic acid modulates angiogenesis. Methods and compositios for binding to the nucleic acid include utilizing RNA interference (RNAi). RNAi is induced by short (e.g., 30 nucleotides) double stranded RNA (dsRNA) molecules which are present in the cell. These short dsRNA molecules, called short interfering RNA (siRNA) cause the destruction of messenger RNAs (mRNAs) which share sequence homology with the siRNA. Beach et al., international publication number WO/2003/062394 published Jul. 31, 2003; McSwiggen et al., U.S. patent publication number 2005/0032733 published Feb. 10, 2005; Cicciarelli et al., U.S. Pat. No. 8,236,771 issued Aug. 7, 2012. In various embodiments, the target nucleic acid sequence encodes a galectin protein or a portion thereof. For example, the RNA interference agent negatively modulates expression of any of galectins 1-11 or a portion thereof (e.g., a carbohydrate binding domain).

Methods for constructing synthetic siRNA or an antisense expression cassette and inserting it into a recombinantly engineered nucleic acid of a vector are well known in the art and are shown for example in Reich et al. U.S. Pat. No. 7,847,090 issued Dec. 7, 2010; Reich et al. U.S. Pat. No. 7,674,895 issued Mar. 9, 2010; Khvorova et al. U.S. Pat. No. 7,642,349 issued Jan. 5, 2010. For example, the invention herein includes synthetic siRNAs that include a sense RNA strand and an antisense RNA strand, such that the sense RNA strand includes a nucleotide sequence substantially identical to a target nucleic acid sequence in cells. Thus, under the circumstances of cells being contacted with viral vectors encoding the siRNAs, the cells express the siRNAs that then negatively modulate expression of the target nucleic acid sequence.

### Galectins

Lectin proteins bind carbohydrates specifically and to agglutinate cells (See international publication number WO/2006/113311 which is incorporated by reference herein in its entirety). Lectins have been shown to be involved in a wide variety of cellular functions including cell-cell and cell-matrix interactions. Lectins are widespread among plants, invertebrates and mammals. Animal lectins have been grouped into families: C-type lectins; P-type lectins; galectins (formerly termed S-type lectins); and pentraxins (see, for example, Barondes et al., J. Biol. Chem. 269:20807, 1994).

Mammalian galectins recognize lactose and related galactosides. While all mammalian galectins share similar affinity for small β-galactosides, they show significant differences in binding specificity for more complex glycoconjugates (Henrick et al., Glycobiology 8:45, 1998; Sato et al., J. Biol. Chem. 267:6983, 1992; and Seetharaman et al., J. Biol. Chem. 273:13047, 1998). In addition to binding β-galactoside sugars, galectins possess hemagglutination activity. Laminin, a naturally occurring glycoprotein containing numerous polylactosamine chains, has been shown to be a natural ligand for certain galectins. Laminin is a component of the basal laminae, the extracellular matrix which underlies all epithelia and surrounds individual muscle, fat and Schwann cells. Interactions between cells and the basal laminae are known to influence the migration and/or differentiation of various cell types during mammalian development. Galectins do not contain traditional sequences that specify membrane translocation, but are both secreted and located intracellularly. In addition to their affinity for β-galactoside sugars, members of the galectin family share significant sequence similarity in the carbohydrate recognition domain (CRD; also referred to as the carbohydrate-binding domain), the relevant amino acid residues of which have been determined by X-ray crystallography (Lobsanov et al., J. Biol. Chem. 267:27034, 1993 and Seetharaman et al., supra). Galectins have been implicated in a wide variety of biological functions including cell adhesion (Cooper et al., J. Cell Biol. 115:1437, 1991), growth regulation (Wells et al., Cell 64:91, 1991), cell migration (Hughes, Curr. Opin. Struct. Biol. 2:687, 1992), neoplastic transformation (Raz et al., Int. J. Cancer 46:871, 1990) and immune responses (Offner et al., J. Neuroimmunol. 28:177, 1990).

### Galectin-1

Galectin-1 forms a homodimer of 14 kilodalton subunits and each subunit has a single binding site. Galectin-1 is synthesized in the cytosol of mammalian cells where the lectin accumulates in a monomeric form (Cummings et al., U.S. Pat. No. 5,948,628 issued Sep. 7, 1999; Cummings et al., U.S. Pat. No. 6,225,071 issued May 1, 2001; Horie et al., U.S. Pat. No. 6,890,531 issued May 10, 2005; and Camby et al, U.S. Pat. No. 7,964,575 issued Jun. 21, 2011).

The amino acid sequence for human galectin-1 (SEQ ID NO: 6) is shown below:

```
    1 MACGLVASNL NLKPGECLRV RGEVAPDAKS FVLNLGKDSN NLCLHFNPRF
  51 NAHGDANTIV CNSKDGGAWG TEQREAVFPF QPGSVAEVCI TFDQANLTVK
101 LPDGYEFKFP NRLNLEAINY MAADGDFKIK CVAFD
```

### Galectin-3

Members of the galectin-3 family of proteins (previously known as CBP-35, Mac-2, L-34, EBP, and RL-29) typically have a sequence of about 240 to 270 amino acids and have molecular weights that from about 25 to about 29 kDa. Galectin-3 proteins are generally composed of a short N-terminal domain, a C-terminal domain which includes a galactoside-binding region, and an intervening proline, glycine, and tyrosine-rich domain which includes repeats of 7-10 conserved amino acids (Liu et al., Biochemisty 35:6073, 1996 and Cherayil et al., Proc. Natl. Acad Sci. USA, 87:7324, 1990). The tandem repeats are similar to those found in the collagen gene superfamily. The number of repeats varies between galectin-3 proteins and accounts for the differences in size between galectin-3 proteins from different species. The N-terminal domain of galectin-3 permits the protein to undergo multimerization upon binding to surfaces containing glycoconjugate ligands.

Galectin-3 is expressed in various inflammatory cells (e.g., activated macrophages, basophils, and mast cells) and in epithelia and fibroblasts of various tissues (Perillo et al., J. Mol. Med. 76:402, 1998). It is found on the cell surface, within the extracellular matrix (ECM), in the cytoplasm, and in the nucleus of cells. On the cell surface or in the ECM galectin-3 is thought to mediate cell-cell and cell-matrix interactions by binding to complementary glycoconjugates containing polylactosamine chains found in many ECM and cell surface molecules. Galectin-3 is thought to inhibit cell-matrix adhesion by binding to laminin. In the nucleus of cells galectin-3 may influence cell-matrix interactions indirectly by influencing the expression of well-known cell adhesion molecules (e.g., a6β1 and α4β7 integrins, Warlfield et al., Invasion Metastasis 17:101, 1997 and Matarrese et al., Int. J. Cancer 85:545, 2000) and cytokines (e.g., IL-1, Jeng et al., Immunol. Lett. 42: 113, 1994). Galectin-3 expression is developmentally regulated in selected organs such as the kidney and its expression level in pulmonary alveolar epithelial cells and hepatocytes is up-regulated following injury. Galectin-3 has been shown to concentrate in the nucleus of certain cell types during proliferation. Expression of galectin-3 is elevated in certain tumors, suggesting galectin-3 plays a role in metastasis. Indeed, overexpression of galectin-3 in a weakly metastatic cell line caused a significant increase in metastatic potential (Raz et al., supra).

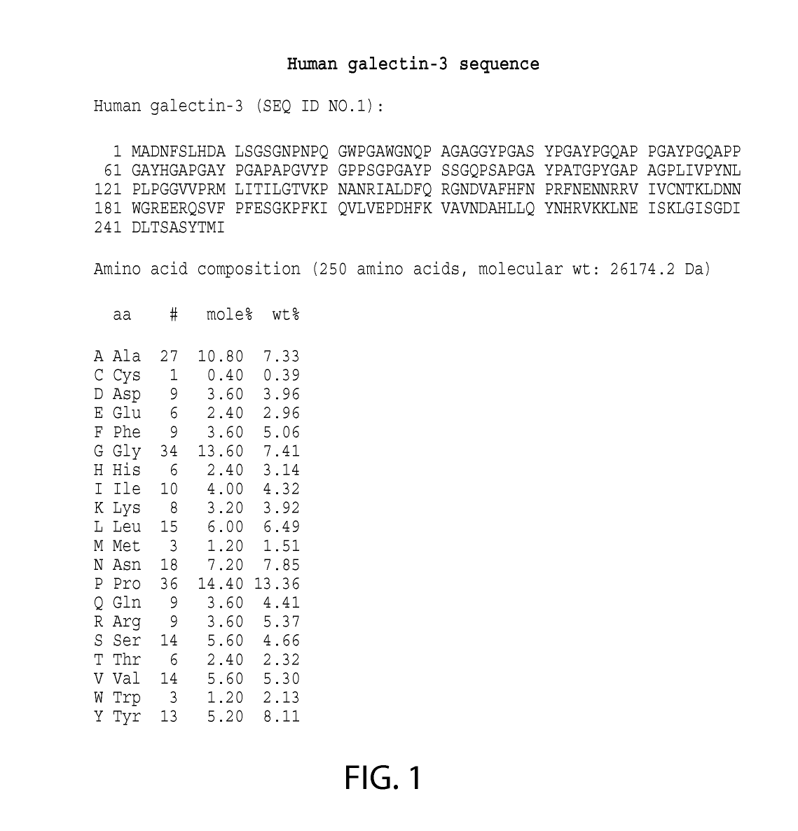

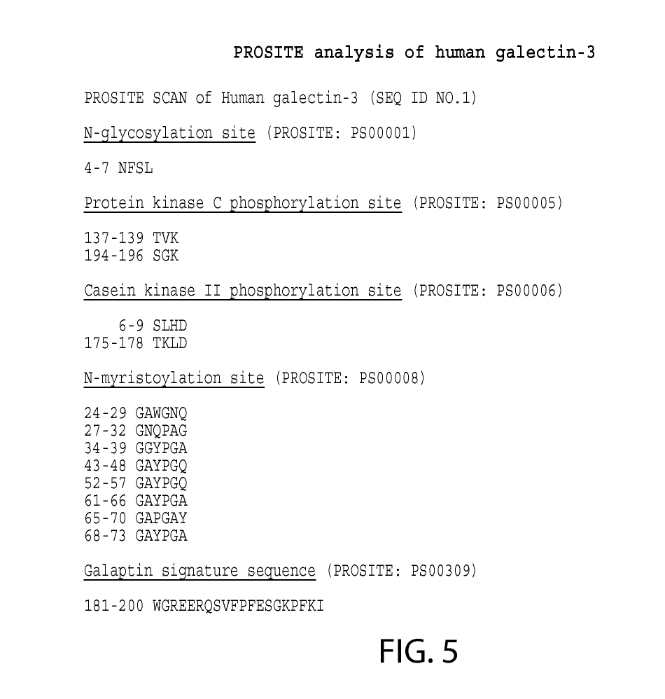

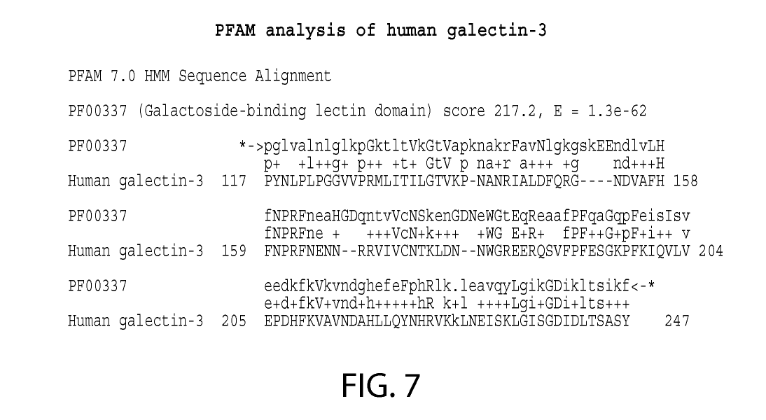

Human galectin-3 is 250 amino acids in length and has an approximate molecular weight of 26.1 kDa (SEQ ID NO: 1, FIG. 1). As illustrated in FIGS. 1, 3, 5, and 7, human galectin-3 contains the following domains, signature sequences, or other structural features (for general information regarding PS and PF prefix identification numbers, see Sonnhammer et al., Protein 28:405, 1997): an N-terminal domain located at about amino acid residues 1 to 14 of SEQ ID NO: 1; a proline, glycine, and tyrosine-rich domain located at about amino acid residues 15 to 116 of SEQ ID NO: 1; a galactoside-binding domain located at about amino acid residues 117 to 247 of SEQ ID NO: 1; a galaptin signature sequence (PROSITE No. PS00309) located at about amino acids 181 to 200 of SEQ ID NO: 1; one potential N-glycosylation site (PROSITE No. PS00001) located at about amino acids 4 to 7 of SEQ ID NO: 1; two potential protein kinase C phosphorylation sites (PROSITE No. PS00005) located at about amino acids 137 to 139 and 194 to 196 of SEQ ID NO: 1; two potential casein kinase II phosphorylation sites (PROSITE No. PS00006) located at about amino acids 6 to 9 and 175 to 178 of SEQ ID NO: 1; and eight potential myristoylation sites (PROSITE No. PS00008) located at about amino acids 24 to 29, 27 to 32, 34 to 39, 43 to 48, 52 to 57, 61 to 66, 65 to 70, and 68 to 73 of SEQ ID NO: 1.

As defined herein, a “galectin-3 protein” includes a galectin-3 “N-terminal domain”, a galectin-3 “proline, glycine, and tyrosine-rich domain”, and/or a galectin-3 “galactoside-binding domain”. These domains are further defined as follows.

As used herein, a galectin-3 “N-terminal domain” includes an amino acid sequence of about 10-20 amino acids, preferably about 14 amino acids that shares at least about 60%, 70%, 80%, 90%, 95%, 99%, or 100% identity with amino acids 1 to 14 of SEQ ID NO: 1.

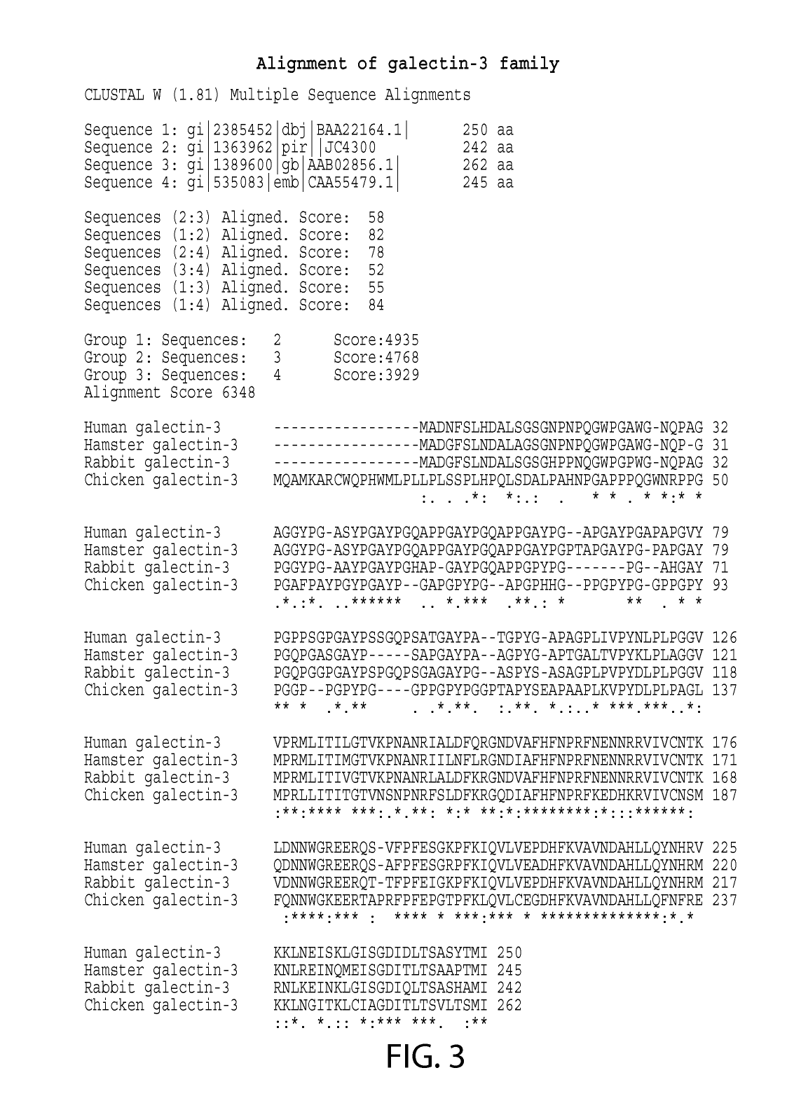

The N-terminal domain can include an N-glycosylation site (PROSITE No. PS00001) and/or a casein kinase 11 phosphorylation site (PROSITE No. PS00006). The PROSITE N-glycosylation site has the consensus sequence: N-{P}-[ST]-{P} and the PROSITE casein kinase II phosphorylation site has the consensus sequence: [ST]-X(2)-[DE]. In the above consensus sequences, and other motifs or signature sequences described herein, the standard IUPAC one-letter code for the amino acids is used. Each element in the pattern is separated by a dash (-); square brackets ([ ]) indicate the particular residues that are accepted at that position; X indicates that any residue is accepted at that position; and numbers in parentheses (( )) indicate the number of residues represented by the accompanying amino acid. In certain embodiments, the N-terminal domain includes amino acids L7 and L11 of SEQ ID NO: 1. As shown in FIG. 3, these amino acids are conserved across several mammalian species of galectin-3 and may therefore play a catalytic and/or structural role.

As used herein, a galectin-3 “proline, glycine, and tyrosine-rich domain” includes an amino acid sequence of about 60 to about 140 amino acids, more preferably about 80 to 120 amino acids, or about 90 to 110 amino acids that shares at least about 60%, 70%, 80%, 90%, 95%, 99%, or 100% identity with amino acids 15 to 116 of SEQ ID NO: 1. The proline, glycine, and tyrosine-rich domain can also include one, two, three, four, five, six, seven, or eight N-myristoylation sites (PROSITE No. PS00008) which have the consensus sequence: G-{EDRKHPFYW}-X(2)-[STAGCN]-{P}. In certain embodiments, the proline, glycine, and tyrosine-rich domain includes the following amino acids and regions of SEQ ID NO: 1: G21, P23, G27, N28, P30, G32, G34, P37, Y41-P46, G53, Y55-G57, P61, G62, G66, P72, G73, G77, Y79-G81, P83, G87, Y89, P90, G99, Y101, P102, P106, Y107, A109, L114, and V116. These amino acids and regions are conserved across several mammalian species of galectin-3 and may play a catalytic and/or structural role (see amino acids indicated with a “*” in FIG. 3).

As used herein, a galectin-3 “galactoside-binding domain” includes an amino acid sequence of about 80 to about 180 amino acids having a bit score for the alignment of the sequence to the consensus sequence PF00337 from PFAM (ID NO: 3) of at least 150. Preferably, a galectin-3 galactoside-binding domain includes at least about 100 to 160 amino acids, more preferably about 110 to 150 amino acids, or about 120 to about 140 amino acids and has a bit score for the alignment of the sequence to the consensus sequence PF00337 from PFAM (SEQ ID NO: 3) of at least 150, at least 175, or 200 or greater.

To calculate the bit score for the alignment of a particular sequence to the consensus sequence PF00337 from PFAM, the sequence of interest can be searched against the PFAM database of HMMs (e.g., the PFAM database, release 2.1) using the default parameters available at www.sanger.ac.uk/Software/Pfam. A description of the PFAM database can be found in Sonnhammer et al., supra and a detailed description of HMMs can be found, for example, in Gribskov et al., Meth. Enzymol. 183:146, 1990 and Stultz et al., Protein Sci. 2:305, 1993.

The galectin-3 galactoside-binding domain can further include one, preferably two, protein kinase C phosphorylation sites (PROSITE No. PS00005); a casein kinase II phosphorylation site (PROSITE No. PS00006); and/or a galaptin signature sequence (PROSITE No. PS00309). The protein kinase C phosphorylation site has the following consensus sequence: [ST]-X-[RK]. The galaptin signature sequence has the following consensus sequence: W-[GEK]-X-[EQ]-X-[KRE]-X(3,6)-[PCTF]-[LIVMF]-[NQEGSKV]-X-[GH]-X(3)-[DENKHS]-[LIVMFC]. In certain embodiments, the galectin-3 galactoside-binding domain includes the following amino acids and regions of ID NO: 1: P117, Y118, L120-L122, G125, P128, R129, L131-1134, G136-V138, N141, N143, R144, L147, F149, R151, G152, D154, A156-F163, E165, R169-N174, N179-G182, E184-R186, F190-E193, G195, P197-K199, Q201-L203, E205, D207-Q220, N222, R224, L228, 1231, 1236, G238-1240, and L242-S244. These amino acids and regions are conserved across several mammalian species of galectin-3 and may play a catalytic and/or structural role (see amino acids indicated with a “*” in FIG. 3).

Certain galectin-3 proteins include the amino acid sequence of human galectin-3 as represented by SEQ ID NO: 1. Other galectin-3 proteins include an amino acid sequence that is substantially identical to the amino acid sequence of SEQ ID NO: 1. The term “substantially identical” is used herein to refer to a first amino acid that contains a sufficient or minimum number of amino acid residues that are identical to aligned amino acid residues in a second amino acid sequence such that the first and second amino acid sequences can have a common structural domain and/or common functional activity. For example, amino acid sequences that contain a common structural domain having at least about 60%, or 65% identity, preferably at least 75% identity, more preferably at least 85%, 90%, 91%, 92%, 93%, 94%, 95%, 96%, 97%, 98%, or 99% identity to SEQ ID NO: 1 are termed substantially identical to the amino acid sequence of SEQ ID NO: 1. In particular, proteins which contain accidentally or deliberately induced alterations, such as deletions, additions, substitutions or modifications of certain amino acid residues of SEQ ID NO: 1 may fall within the definition of galectin-3 proteins provided herein. It will also be appreciated that as defined herein, galectin-3 proteins may include regions represented by the amino acid sequence of galectin-3 taken from other mammalian species including but not limited to bovine, canine, feline, caprine, ovine, porcine, murine, and equine species.

Calculations of sequence identity between sequences are performed as follows. To determine the percent identity of two amino acid sequences, the sequences are aligned for optimal comparison purposes (e.g., gaps can be introduced in one or both of a first and a second amino acid sequence for optimal alignment). The amino acid residues at corresponding amino acid positions or nucleotide positions are then compared. When a position in the first sequence is occupied by the same amino acid residue or nucleotide as the corresponding position in the second sequence, then the proteins are identical at that position. The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using an alignment software program using the default parameters. Suitable programs include, for example, CLUSTAL W by Thompson et al., Nuc. Acids Research 22:4673, 1994 (www.ebi.ac.uk/clustalw), BL2SEQ by Tatusova and Madden, FEMS Microbiol. Lett. 174:247, 1999 (www.ncbi.nlm.nih.gov/blast/bl2seq/bl2.html), SAGA by Notredame and Higgins, Nuc. Acids Research 24:1515, 1996 (igs-server.cnrs-mrs.fr/-cnotred), and DIALIGN by Morgenstern et al., Bioinformatics 14:290, 1998 (bibiserv.techfak.uni-bielefeld.de/dialign).

### Galectin-7

Members of the galectin-7 family of proteins typically exist as monomers that include between about 130 to about 140 amino acids and have molecular weights between about 15 and about 16 kDa (see, for example, Magnaldo et al., Develop. Biol. 168:259, 1995 and Madsen et al., J. Biol. Chem. 270:5823, 1995). Expression of galectin-7 has been associated with the onset of epithelial stratification (Timmons et al., Int. J. Dev. Biol. 43:229, 1999). Galectin-7 is thought to play a role in cell-matrix and cell-cell interactions. Galectin-7 is found in areas of cell-cell contact (e.g., in the upper layers of human epidermis); its expression is sharply downregulated in anchorage independent keratinocytes and it is absent in a malignant keratinocyte cell line. Galectin-7 may be required for the maintenance of normal keratinocytes (see, Madsen et al., supra).

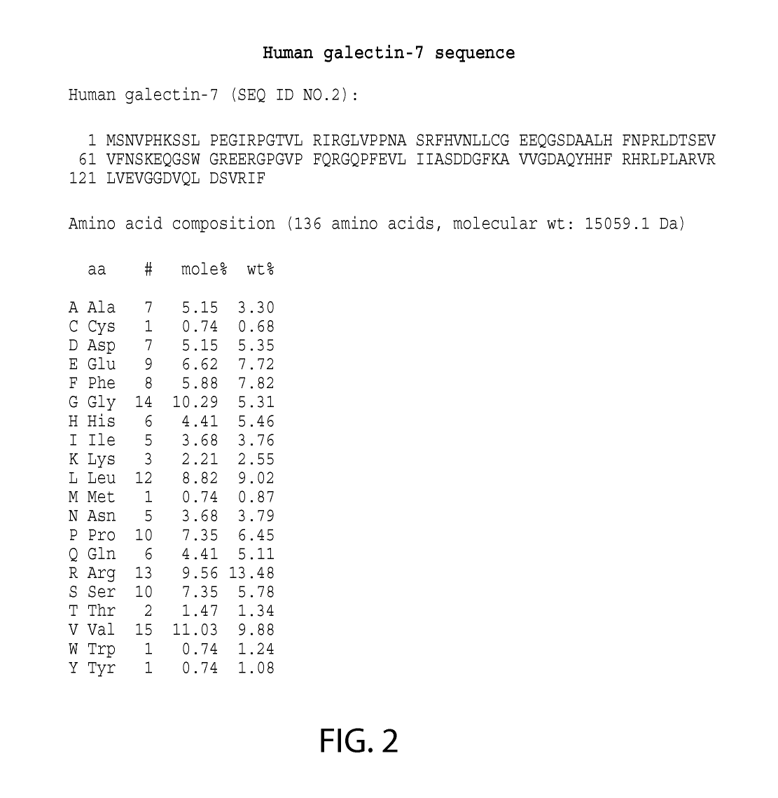

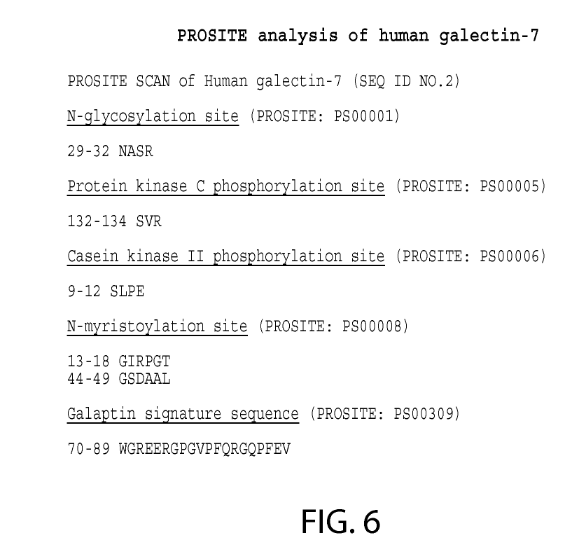

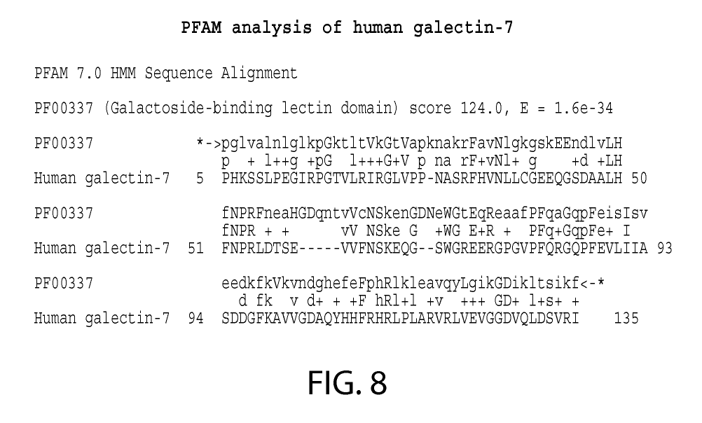

Human galectin-7 includes 136 amino acids and has an approximate molecular weight of 15.1 kDa (SEQ ID NO: 2, FIG. 2). As illustrated in FIGS. 2, 4, 6, and 8, human galectin-7 contains the following domains, signature sequences, or other structural features: a galactoside-binding domain located at about amino acid residues 5 to 135 of SEQ ID NO: 2; a galaptin signature sequence (PROSITE No. PS00309) located at about amino acids 70 to 89 of SEQ ID NO: 2; one N-glycosylation site (PROSITE No. PS00001) located at about amino acids 29 to 32 of SEQ ID NO: 2; one protein kinase C phosphorylation site (PROSITE No. PS00005) located at about amino acid positions 132 to 134 of SEQ ID NO: 2; one casein kinase II phosphorylation site (PROSITE No. PS00006) located at about amino acids 9 to 12 of SEQ ID NO: 2; and two myristoylation sites (PROSITE No. PS00008) located at about amino acids 13 to 18 and 44 to 49 of SEQ ID NO: 2.

As defined herein, a “galectin-7 protein” includes a galectin-7 “galactoside-binding domain”. This domain is further defined as follows.

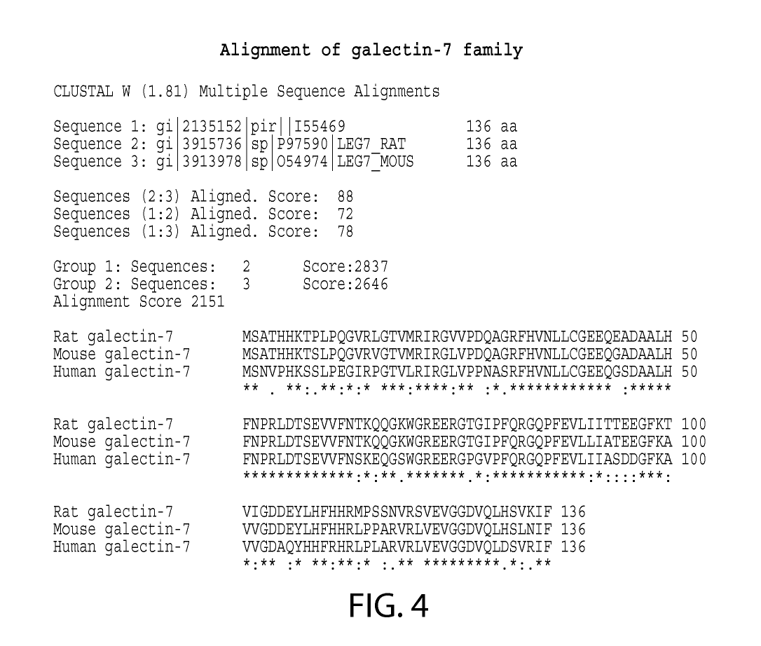

As used herein, a galectin-7 “galactoside-binding domain” includes an amino acid sequence of about 80 to about 180 amino acids having a bit score for the alignment of the sequence to the consensus sequence PF00337 from PFAM (SEQ ID NO: 3) of at least 80. Preferably, a galectin-7 galactoside-binding domain includes at least about 100 to 160 amino acids, or about 110 to 150 amino acids, or about 120 to 140 amino acids and has a bit score for the alignment of the sequence to the consensus sequence PF00337 from PFAM (SEQ ID NO: 3) of at least 80, more preferably at least 100, most preferably 120 or greater. The galectin-7 galactoside-binding domain can include one N-glycosylation site (PROSITE No. PS00001); one protein kinase C phosphorylation site (PROSITE No. PS00005); one casein kinase II phosphorylation site (PROSITE No. PS00006); one or two myristoylation sites (PROSITE No. PS00008); and/or a galaptin signature sequence (PROSITE No. PS00309). In certain embodiments, the galectin-7 galactoside-binding domain includes the following amino acids and regions of SEQ ID NO: 2: M1, S2, H6, K7, L10, P11, G13, R15, G17-V19, R21-G24, V26, P27, A30, R32-Q43, D46-N63, K65, Q67, G68, W70-G76, G78, P80-L90, 192, G97-K99, V101, G103, D104, Y107, H109, F110, H112, R113, P115, V119, R120, V122-L130, S132, I135, and F136. These amino acids and regions are conserved across several mammalian species of galectin-7 and may play a catalytic and/or structural role (see amino acids indicated with a “*” in FIG. 4).

Certain galectin-7 proteins include the amino acid sequence of human galectin-7 as represented by SEQ ID NO: 2. Other galectin-7 proteins include an amino acid sequence that is substantially identical to the amino acid sequence of SEQ ID NO: 2. In particular, proteins which contain accidentally or deliberately induced alterations, such as deletions, additions, substitutions or modifications of certain amino acid residues of SEQ ID NO: 2 may fall within the definition of galectin-7 herein. It will also be appreciated that as defined herein, galectin-7 proteins may include regions represented by the amino acid sequence of galectin-7 taken from other mammalian species including but not limited to bovine, canine, feline, caprine, ovine, porcine, murine, and equine species.

### Galectin-8

Galectin-8 is a widely expressed protein, present for example, in liver, heart, muscle, kidney, spleen, hind-limb and brain, and the sequence of human and rat galectin-8 genes and proteins are available (see for example Hadari, et al., Trends in Glycosci and Glycotechnol. 9: 103-112, 1997). The highly hydrophilic character and function for binding to Gal(β1-4GlcNAC disaccharides found in the O-linked oligosaccharides of mucins make this protein an ideal agent for treating dry eye syndrome.

Alternative forms of amino acid sequence for human galectin-8 are known for example, a 316 amino acid form (Accession number O00214, created 1 Nov. 1997) and a 359 amino acid form (Accession number Q8TEV1, created 1 Jun. 2002). These sequences, while similar or identical for significant lengths, are not overall mere length variants, having portions of difference. The 316 form amino acid sequence, using the one letter amino acid code, is shown below (SEQ ID NO: 4):

MLSLNNLQNI IYNPVIPYVG TIPDQLDPGT LIVICGHVPS
DADRFQVDLQ NGSSVKPRAD 60
VAFHFNPRFK RAGCIVCNTL INEKWGREEI TYDTPFKREK
SFEIVIMVLK DKFQVAVNGK 120
HTLLYGHRIG PEKIDTLGIY GKVNIHSIGF SFSSDLQSTQ
ASSLELTEIS RENVPKSGTP 180
QLSLPFAARL NTPMGPGRTV VVKGEVNANA KSFNVDLLAG
KSKDIALHLN PRLNIKAFVR 240
NSFLQESWGE EERNITSFPF SPGMYFEMII YCDVREFKVA
VNGVHSLEYK HRFKELSSID 300
TLEINGDIHL LEVRSW 316

The amino acid sequence of the longer form is shown below (SEQ ID NO: 5):

MMLSLNNLQN IIYSPVIPYV GTIPDQLDPG TLIVICGHVP
SDADRFQVDL QNGSSVKPRA 60
DVAFHFNPRF KRAGCIVCNT LINEKWGREE ITYDTPFKRE
KSFEIVIMVL KDKFQVAVNG 120
KHTLLYGHRI GPEKIDTLGI YGKVNI SIG FSFSSDLQST
QASSLELTEI SRENVPKSGT 180
PQLPSNRGGD ISKIAPRTVY TKSKDSTVNH TLTCTKIPPT
NYVSKILPFA ARLNTPMGPG 240
GTVVVKGEVN ANAKSFNVDL LAGKSKHIAL HLNPRLNIKA
FVRNSFLQES WGEEERNITS 300
FPFSPGMYFE MIIYCDVREF KVAVNGVHSL EYKHRFKELS
SIDTLEINGD IHLLEVRSW 359

As defined herein, a “galectin-8 protein” may include a galectin-8 “N-terminal domain”, a galectin-8 “proline, glycine, and tyrosine-rich domain”, and/or a galectin-8 “galactoside-binding domain”. These domains are further defined as follows.

As used herein, a galectin-8 “N-terminal domain” includes an amino acid sequence of about 10-20 amino acids, preferably about 14 amino acids that shares at least about 60%, 70%, 80%, 90%, 95%, 99%, or 100% identity with amino acids 1 to 14 of SEQ ID NOs:4 or 5. The N-terminal domain can include an N-glycosylation site (PROSITE No. PS00001) and/or a casein kinase II phosphorylation site (PROSITE No. PS00006). The PROSITE N-glycosylation site has the consensus sequence: N-{P}-[ST]-{P} and the PROSITE casein kinase II phosphorylation site has the consensus sequence: [ST]-X(2)-[DE]. In the above consensus sequences, and other motifs or signature sequences.

As used herein, a galectin-8 “proline, glycine, and tyrosine-rich domain” includes an amino acid sequence of about 60 to 140 amino acids, more preferably about 80 to 120 amino acids, or about 90 to 110 amino acids that shares at least about 60%, 70%, 80%, 90%, 95%, 99%, or 100% identity with amino acids 15 to 116 of each of SEQ ID NOs: 4 and 5. The proline, glycine, and tyrosine-rich domain can also include one, two, three, four, five, six, seven, or eight N-myristoylation sites (PROSITE No. PS00008) which have the consensus sequence: G-{EDRKHPFYW}-X(2)-[STAGCN]-{P}. In certain embodiments, the proline, glycine, and tyrosine-rich domain includes the following amino acids and regions of SEQ ID NO: 4: G20, P23, P28, G29, G36, P39, and other such residues as are obvious to one of skill in the art. These amino acids and regions are conserved across several mammalian species of galectin-8 and may play a catalytic and/or structural role. In certain embodiments, the proline, glycine, and tyrosine-rich domain includes the following amino acids and regions of SEQ ID NO:5: G21, P24, P29, G30, G37, P40, and other such residues as are obvious to one of skill in the art.

As used herein, a galectin-4 “galactoside-binding domain” includes an amino acid sequence of about 80 to 180 amino acids having a bit score for the alignment of the sequence to the consensus sequence PF00337 from PFAM (SEQ ID NO: 3) of at least 150. Preferably, a galectin-3 galactoside-binding domain includes at least about 100 to 160 amino acids, more preferably about 110 to 150 amino acids, or about 120 to 140 amino acids and has a bit score for the alignment of the sequence to the consensus sequence PF00337 from PFAM (SEQ ID NO: 3) of at least 150, more preferably at least 175, most preferably 200 or greater.

To calculate the bit score for the alignment of a particular sequence to the consensus sequence PF00337 from PFAM, the sequence of interest can be searched against the PFAM database of HMMs (e.g., the PFAM database, release 2.1) using the default parameters available at www.sanger.ac.uk/Software/Pfam. A description of the PFAM database can be found in Sonnhammer et al., supra and a detailed description of HMMs can be found, for example, in Gribskov et al., Meth. Enzymol. 183:146, 1990 and Stultz et al., Protein Sci. 2:305, 1993.

A galectin-8 galactoside-binding domain can further include one or two protein kinase C phosphorylation sites (PROSITE No. PS00005); a casein kinase II phosphorylation site (PROSITE No. PS00006); and/or a galaptin signature sequence (PROSITE No. PS00309). The protein kinase C phosphorylation site has the following consensus sequence: [ST]-X-[RK]. The galaptin signature sequence has the following consensus sequence: W-[GEK]-X-[EQ]-X-[KRE]-X(3,6)-[PCTF]-[LIVMF]-[NQEGSKV]-X-[GH]-X(3)-[DENKHS]-[LIVMFC]. In certain embodiments, the galectin-8 galactoside-binding domain includes the following amino acids and regions of SEQ ID NO: 4: L123-L124, G126, P131, R128, L140-1146, and other sites similar to those as demonstrated above. These amino acids and regions are conserved across several mammalian species of galectin-8 and may play a catalytic and/or structural role (see amino acids indicated with a “*” in FIG. 3).

Certain galectin-8 proteins include the amino acid sequence of human galectin-8 as represented by SEQ ID NOs: 4 and 5. Other galectin-8 proteins include an amino acid sequence that is substantially identical to the amino acid sequence of SEQ ID NOs: 4 or 5. The term “substantially identical” is used herein to refer to a first amino acid that contains a sufficient or minimum number of amino acid residues that are identical to aligned amino acid residues in a second amino acid sequence such that the first and second amino acid sequences can have a common structural domain and/or common functional activity. For example, amino acid sequences that contain a common structural domain having at least about 60%, or 65% identity, preferably at least 75% identity, more preferably at least 85%, 90%, 91%, 92%, 93%, 94%, 95%, 96%, 97%, 98%, or 99% identity to SEQ ID NOs:4 or 5 are termed substantially identical to the amino acid sequence of SEQ ID NOs: 4 or 5. In particular, proteins which contain accidentally or deliberately induced alterations, such as deletions, additions, substitutions or modifications of certain amino acid residues of SEQ ID NOs: 4 or may fall within the definition of galectin-8 proteins herein. It will also be appreciated that as defined herein, galectin-8 proteins may include regions represented by the amino acid sequence of galectin-8 taken from other mammalian species including but not limited to bovine, canine, feline, caprine, ovine, porcine, murine, and equine species.

The material in computer readable form ASCII text file (13 kilobytes) created May 14, 2015 entitled “34724153_SEQ_ID_{—}05152015”, containing sequence listings numbers 1-6 (described supra), has been electronically filed herewith and is incorporated by reference herein in its entirety.

### Preparation of Galectin Proteins

It will be appreciated by one of ordinary skill in the art, that the galectins of this invention can be obtained from any available source. These include but are not limited to proteins isolated from natural sources, produced recombinantly or produced synthetically, e.g., by solid phase procedures. In accordance with the present invention, polynucleotide sequences which encode galectin-3, galectin-7 or galectin-8 may be used in recombinant DNA molecules that direct the expression of the galectins of this invention in appropriate host cells. Cherayil et al., supra, Madsen et al., supra, and Hadri et al., supra describe in detail the cloning of human galectin-1, -3, -7 and -8 respectively. In order to express a biologically active galectin-1, galectin-3, galectin-7 or galectin-8, the nucleotide sequence encoding galectin-1, galectin-3, galectin-7, galectin-8 or their functional equivalent, is inserted into an appropriate expression vector, i.e., a vector which contains the necessary elements for the transcription and translation of the inserted coding sequence. Methods which are well known to those skilled in the art can be used to construct expression vectors containing a galectin-1-encoding, galectin-3-encoding, galectin-7-encoding or galectin-8-encoding sequence and appropriate transcriptional or translational controls. These methods include in vitro recombinant DNA techniques, synthetic techniques and in vivo recombination or genetic recombination. The introduction of deletions, additions, or substitutions is achieved using any known technique in the art e.g., using PCR based mutagenesis. Such techniques are described in Sambrook et al., Molecular Cloning: A Laboratory Manual, Cold Spring Harbor Press, Plainview, N.Y., 1989 and Ausubel et al., Current Protocols in Molecular Biology, John Wiley & Sons, New York, N.Y., 1989. A variety of expression vector/host systems may be utilized to contain and express a galectin-1-encoding, galectin-3-encoding, galectin-7-encoding or galectin-8-encoding sequence. These include but are not limited to microorganisms such as bacteria transformed with recombinant bacteriophage, plasmid or cosmid DNA expression vectors; yeast transformed. with yeast expression vectors; insect cell systems infected with virus expression vectors (e.g., baculovirus); plant cell systems transfected with virus expression vectors (e.g., cauliflower mosaic virus, CaMV; tobacco mosaic virus, TMV) or transformed with bacterial expression vectors (e.g., Ti, pBR322, or pET25b plasmid); or animal cell systems. Alternatively, the galectins are produced using chemical methods to synthesize a galectin-1, galectin-3, galectin-7 or galectin-8 amino acid sequence, whole or in part. For example, peptide synthesis can be performed using various solid-phase techniques (Roberge et al., Science 269:202, 1995) and automated synthesis may be achieved, for example, using the 431A peptide synthesizer (available from Applied Biosystems of Foster City, Calif.) in accordance with the instructions provided by the manufacturer.

### Pharmaceutical Compositions

In one aspect of the present invention, pharmaceutical compositions are provided, such that these compositions comprise at least one inhibitor of an activity of a galectin protein (e.g, a galectin-1 protein, a galectin-3 protein, a galectin-7 protein, and a galectin-8 protein), and optionally comprise a pharmaceutically acceptable carrier. In certain embodiments, these compositions optionally further comprise one or more additional therapeutic agents. In certain embodiments, the additional therapeutic agent or agents are selected from the group consisting of growth factors, anti-inflammatory agents, vasopressor agents, collagenase inhibitors, topical steroids, matrix metalloproteinase inhibitors, ascorbates, angiotensin II, angiotensin III, calreticulin, tetracyclines, fibronectin, collagen, thrombospondin, transforming growth factors (TGF), keratinocyte growth factor (KGF), fibroblast growth factor (FGF), insulin-like growth factors (IGF), vascular endothelial growth factor (VEGF), epidermal growth factor (EGF), platelet derived growth factor (PDGF), neu differentiation factor (NDF), hepatocyte growth factor (HGF), B vitamins such as biotin, and hyaluronic acid.

The phrases “pharmaceutically acceptable carrier” and “pharmaceutically suitable carrier” are used interchangeably herein and include any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical Sciences Ed. by Gennaro, Mack Publishing, Easton, Pa., 1995 discloses various carriers used in formulating pharmaceutical compositions and known techniques for the preparation thereof. Some examples of materials which can serve as pharmaceutically acceptable carriers include, but are not limited to, sugars such as glucose, and sucrose; starches such as corn starch and potato starch; cellulose and its derivatives such as sodium carboxymethyl cellulose, ethyl cellulose, and cellulose acetate; powdered tragacanth; malt; gelatin; talc; excipients such as cocoa butter and suppository waxes; oils such as peanut oil, cottonseed oil, safflower oil, sesame oil, olive oil, corn oil, and soybean oil; glycols; such a propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

### Therapeutically Effective Dose

In yet another aspect, according to the methods of treatment of the present invention, the treatment of ocular angiogenesis (e.g., an angiogenesis-related disorder or condition) or ocular fibrosis (e.g. a fibrosis-related disorder or condition) by contacting the eye with a pharmaceutical composition, as described herein. Thus, the invention provides methods for the treatment of the ocular angiogenesis-related disorder or the fibrosis related disorder comprising administering a therapeutically effective amount of a pharmaceutical composition comprising active agents that inhibit galectin-3, galectin-7 and/or galectin-8 to a subject in need thereof, in such amounts and for such time as is necessary to achieve the desired result. It will be appreciated that this encompasses administering an inventive pharmaceutical as a therapeutic measure to inhibit ocular angiogenesis or ocular fibrosis, or as a prophylactic measure to minimize complications associated with an ocular angiogenesis-associated disorder or with an ocular fibrosis-associated disorder such as age-related macular degeneration, ocular histoplasmosis syndrome, neovascular glaucoma, retrolental fibroplasia, pathologic myopia, angioid streaks, idiopathic disorders, choroiditis, choroidal rupture, overlying choroid nevi, graft rejection, herpes simplex keratitis, leishmaniasis, onchocerciasis, and certain inflammatory diseases. In certain embodiments a “therapeutically effective amount” of the pharmaceutical composition is that amount effective for modulating the angiogenesis-related disorder or fibrosis-related disorder, specifically down-regulating the ocular angiogenesis or ocular fibrosis. The compositions, according to the method of the present invention, may be administered using any amount and any route of administration effective for treating the eye or other tissue. The exact dosage is chosen by the individual physician in view of the patient to be treated. Dosage and administration are adjusted to provide sufficient levels of the active agent(s) or to maintain the desired effect. Additional factors which may be taken into account include the severity of the disease state, e.g., extent of angiogenesis or extent of fibrosis, history of the condition; age, weight and gender of the patient; diet, time and frequency of administration; drug combinations; reaction sensitivities; and tolerance/response to therapy. Long acting pharmaceutical compositions might be administered several times a day, every day, 3 to 4 days, every week, or once every two weeks depending on half-life and clearance rate of the particular composition.

The active agents of the invention are preferably formulated in dosage unit form for ease of administration and uniformity of dosage. The expression “dosage unit form” as used herein refers to a physically discrete unit of active agent appropriate for the patient to be treated. It will be understood, however, that the total daily usage of the compositions will be decided by the attending physician within the scope of sound medical judgment. For any active agent, the therapeutically effective dose can be estimated initially either in cell culture assays or in animal models, usually mice, rabbits, dogs, or pigs. The animal model is also used to achieve a desirable concentration range and route of administration. While direct application to the eye is envisioned as the route of administration, such information can then be used to determine useful doses and additional routes for administration in humans. A therapeutically effective dose refers to that amount of active agent that ameliorates the symptoms or condition. Therapeutic efficacy and toxicity of active agents can be determined by standard pharmaceutical procedures in cell cultures or experimental animals, e.g., ED50 (the dose is therapeutically effective in 50% of the population) and LD50 (the dose is lethal to 50% of the population). The dose ratio of toxic to therapeutic effects is the therapeutic index, and it can be expressed as the ratio, LD50/ED50. Pharmaceutical compositions that exhibit large therapeutic indices are preferred. The data obtained from cell culture assays and animal studies is used in formulating a range of dosage for human use.

### Administration of Pharmaceutical Compositions

After formulation with an appropriate pharmaceutically acceptable carrier in a desired dosage, the pharmaceutical compositions of this invention can be administered to humans and other mammals topically such as ocularly (as by gels, ointments, or drops), i.e., as applied directly to external tissues of the eye. Alternative and additional routes such as injection into the eye including invitreally, subtenonally, and subretinally, or orally, rectally, parenterally, intracisternally, intravaginally, intraperitoneally, bucally, or nasally, depending on the severity of the condition being treated, are envisioned. Ocular injections include intra-ocular injection into the aqueous or the vitreous humor, or injection into the external layers of the eye, such as via subconjunctival injection or subtenon injection. Oral administration is envisioned as effective for synthetic small molecule inhibitors.

Liquid dosage forms for ocular administration include buffers and solubilizing agents, preferred diluents such as water, preservatives such as thymosol, and one or more biopolymers or polymers for conditioning the solution, such as polyethylene glycol, hydroxypropylmethylcellulose, sodium hyaluronate, sodium polyacrylate or tamarind gum.

Liquid dosage forms for oral administration include, but are not limited to, pharmaceutically acceptable emulsions, microemulsions, solutions, suspensions, syrups and elixirs. In addition to the active agent(s), the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Dosage forms for topical or transdermal administration of an inventive pharmaceutical composition include ointments, pastes, creams, lotions, gels, powders, solutions, sprays, inhalants, or patches. The active agent is admixed under sterile conditions with a pharmaceutically acceptable carrier and any needed preservatives or buffers as may be required. For example, ocular or cutaneous infections may be treated with aqueous drops, a mist, an emulsion, or a cream. Administration may be therapeutic or it may be prophylactic. Prophylactic formulations may be present or applied to the site of potential wounds, or to sources of wounds, such as contact lenses, contact lens cleaning and rinsing solutions, containers for contact lens storage or transport, devices for contact lens handling, eye drops, surgical irrigation solutions, ear drops, eye patches, and cosmetics for the eye area, including creams, lotions, mascara, eyeliner, and eyeshadow. The invention includes ophthalmological devices, surgical devices, audiological devices or products which contain disclosed compositions (e.g., gauze bandages or strips), and methods of making or using such devices or products. These devices may be coated with, impregnated with, bonded to or otherwise treated with a disclosed composition.

The ointments, pastes, creams, and gels may contain, in addition to an active agent of this invention, excipients such as animal and vegetable fats, oils, waxes, paraffins, starch, tragacanth, cellulose derivatives, polyethylene glycols, silicones, bentonites, silicic acid, talc, zinc oxide, or mixtures thereof.

Powders and sprays can contain, in addition to the agents of this invention, excipients such as talc, silicic acid, aluminum hydroxide, calcium silicates, polyamide powder, or mixtures of these substances. Sprays can additionally contain customary propellants such as chlorofluorohydrocarbons.

Transdermal patches have the added advantage of providing controlled delivery of the active ingredients to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables. The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use. In order to prolong the effect of an active agent, it is often desirable to slow the absorption of the agent from subcutaneous or intramuscular injection. Delayed absorption of a parenterally administered active agent may be accomplished by dissolving or suspending the agent in an oil vehicle. Injectable depot forms are made by forming microencapsule matrices of the agent in biodegradable polymers such as polylactide-polyglycolide. Depending upon the ratio of active agent to polymer and the nature of the particular polymer employed, the rate of active agent release can be controlled. Examples of other biodegradable polymers include poly(orthoesters) and poly(anhydrides). Depot injectable formulations are also prepared by entrapping the agent in liposomes or microemulsions which are compatible with body tissues.

Solid dosage forms for oral administration include capsules, tablets, pills, powders, and granules. In such solid dosage forms, the active agent is mixed with at least one inert, pharmaceutically acceptable excipient or carrier such as sodium citrate or dicalcium phosphate and/or a) fillers or extenders such as starches, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof.

Solid compositions of a similar type may also be employed as fillers in soft and hard-filled gelatin capsules using such excipients as milk sugar as well as high molecular weight polyethylene glycols and the like. The solid dosage forms of tablets, dragees, capsules, pills, and granules can be prepared with coatings and shells such as enteric coatings, release controlling coatings and other coatings well known in the pharmaceutical formulating art. In such solid dosage forms the active agent(s) may be admixed with at least one inert diluent such as sucrose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active agent(s) only, or preferentially, in a certain part of the intestinal tract, optionally, in a delayed manner. Examples of embedding compositions which can be used include polymeric substances and waxes.

### Uses of Pharmaceutical Compositions

As discussed above and described in greater detail in the Examples, an inhibition of at least one of galectin-3, galectin-7 and galectin-8 are useful to treat or modulate angiogenesis or fibrosis conditions by binding VEGF receptors of the eye, by binding to TGF-β receptors of the eye, by binding to other receptors or modulatory proteins or by binding to oligosaccharide chains of secretory mucins to the transmembrane muscins (or other glycoproteins) without being limited by any particular theory or mechanism of action. In general, it is believed that these inhibitors of galectins will be clinically useful in suppressing development of angiogenic or fibrotic disorders of the eye including for example conditions associated with excessive neovascularization, angiogenesis, or fibrosis.

In general, it is shown herein that these inhibitors of galectins are clinically useful in suppressing angiogenesis or fibrosis associated with any epithelial or endothelial tissue including but not limited to the corneal epithelium; the retinal epithelium, the endothelium of ocular blood and lymph vessels.

Pharmaceutical compositions containing an inhibitor of at least one of any of galectins 1-11 (e.g., a galectin-1, a galectin-3, a galectin-7 and a galectin-8) are, for example herein, useful to promote a normal level of angiogenesis or fibrosis.

Inhibitors of galectins-1, -3, -7 and/or galectin-8 can be administered prophylactically to reduce or prevent damage by excess angiogenesis or fibrosis to the eyes caused by various pathological states. For example, galectins-1, -3, -7 and/or galectin-8 in appropriate amounts used to promote the repair of alveoli and bronchiolar epithelium to prevent, attenuate, or treat acute or chronic lung damage can be controlled by compositions herein. Emphysema, which results in the progressive loss of alveoli, and inhalation injuries, i.e., resulting from smoke inhalation and burns, that cause necrosis of the bronchiolar epithelium and alveoli can be effectively treated using inhibitors of galectins-1, -3, -7 and/or galectin-8, as can damage attributable to chemotherapy, radiation treatment, lung cancer, asthma, black lung and other lung damaging conditions, following galectin treatment.

All animal treatments described in these examples conformed to the Association for Research in Vision and Ophthalmology Resolution on the Use of Animals in Vision Research and the recommendations of the NIH Guide for the Care and Use of Laboratory Animals.

The various embodiments of the invention are exemplified by the following claims and examples and figures are exemplary only and are not to be construed as further limiting. Appendices A, B, and C are hereby incorporated herein by reference in their entireties. The contents of all references including non-patent literature references, issued patents and published patent applications cited in this application are hereby incorporated by reference in their entireties.

### EXAMPLES

### Example 1

### Materials and instruments for synthesis of bis(3-deoxy-3-(3-fluorophenyl-1H-1,2,3-triazol-1-yl)-β-D-galactopyranosyl) sulfane

Bis(3-deoxy-3-(3-fluorophenyl-1H-1,2,3-triazol-1-yl)-β-D-galactopyranosyl) sulfane (TD139) was provided by Profs. Hakon Leffler and Ulf Nilsson (Lund University), and was prepared using the materials and methods described herein.

Melting points were recorded on a Kofler apparatus (Reichert) and are uncorrected. Proton nuclear magnetic resonance (1H) spectra were recorded using a Bruker DRX 400 (400 MHz) or a Bruker ARX 300 (300 MHz) spectrometer; multiplicities are quoted as singlet (s), doublet (d), doublet of doublets (dd), triplet (t), apparent triplet (at) or apparent triplet of doublets (atd). Carbon nuclear magnetic resonance (13C) spectra were recorded using a Bruker DRX 400 (100.6 MHz) spectrometer. Spectra were assigned using COSY, HMQC and DEPT experiments. All chemical shifts are quoted on the d-scale in parts per million (ppm).

Low- and high-resolution (FAB-HRMS) fast atom bombardment mass spectra were recorded using a JEOL SX-120 instrument and low- and high-resolution (ES-HRMS) were recorded with a Micromass Q-TOF instrument. Optical rotations were measured on a Perkin-Elmer 341 polarimeter with a path length of 1 dm; concentrations are given in g per 100 mL. Thin layer chromatography (TLC) was performed using Merck Kieselgel sheets, pre-coated with 60F254 silica. Plates were developed using 10% sulfuric acid. Flash column chromatography was performed with silica (Matrex, 60 Å, 35-70 m, Grace Amicon). Acetonitrile was distilled from calcium hydride and stored over 4 Å molecular sieves. DMF was distilled from 4 Å molecular sieves and stored over 4 Å molecular sieves.

Bis(3-deoxy-3-(3-fluorophenyl-1H-1,2,3-triazol-1-yl)-β-D-galactopyranosyl) sulfane (TD139) was prepared in accordance with the reaction scheme 1 below:

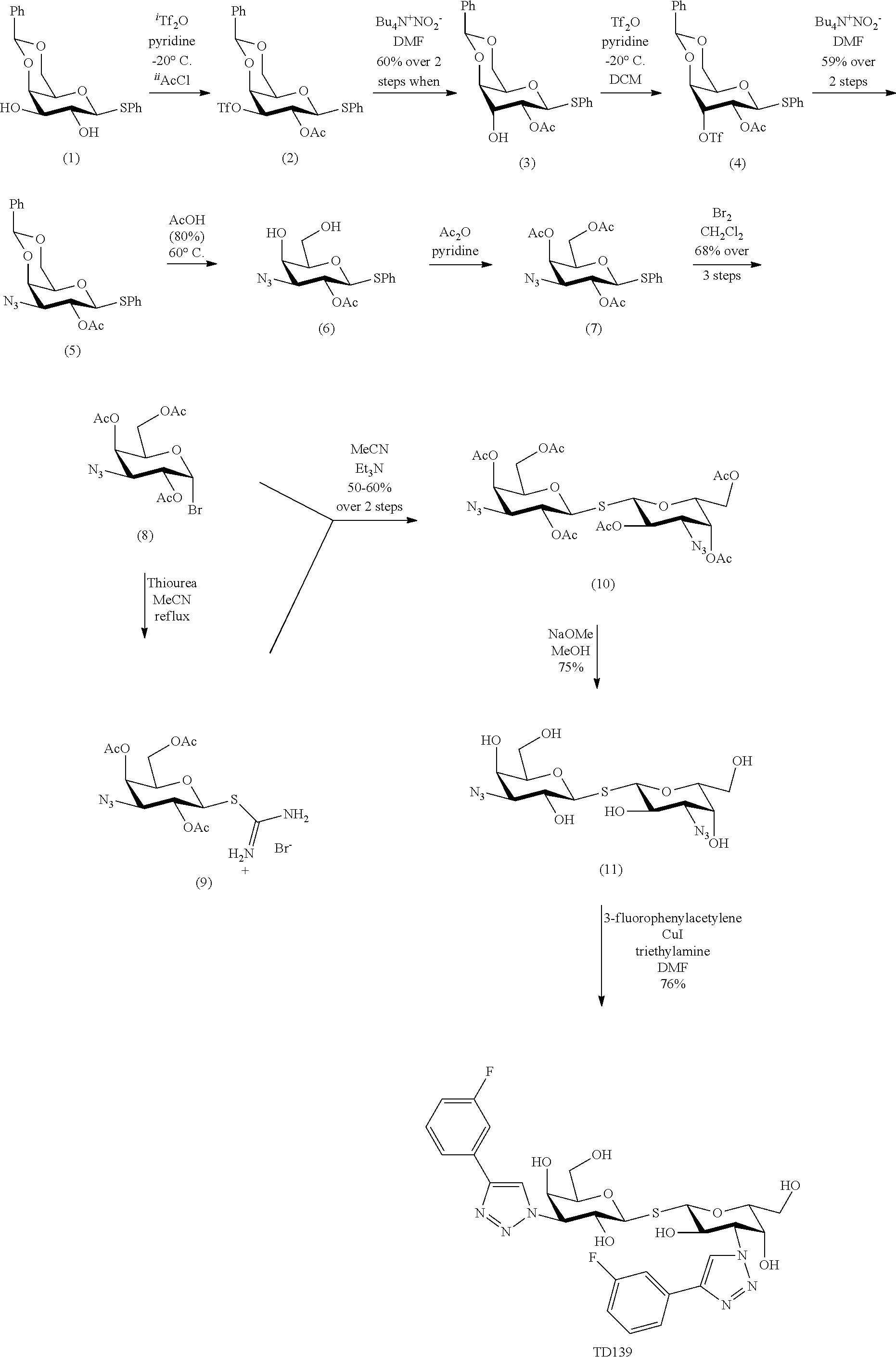

Compound 1 (reaction 1 above) was obtained from Carbosynth Limited 8 & 9 Old Station Business Park—Compton—Berkshire—RG20 6NE—UK or synthesized in three near-quantitative steps from D-galactose, (see Li, Z. and Gildersleeve, J. J. Am. Chem. Soc. 2006, 128, 11612-11619).

### Example 2

### Synthesis of Phenyl 2-O-acetyl-4,6-O-benzylidene-1-thio-3-O-trifluoromethanesulfonyl-β-D-galactopyranoside (Structure 2 in Scheme 1)

Compound 1 (10.5 grams, 29.2 mmol) was dissolved in dried pyridine (4.73 mL, 58.4 mmol) and dried CH₂Cl₂ (132 mL). The reaction mixture was cooled, with stirring, to −20° C. (ice and NaCl bath 3:1). Slowly and under N₂ atmosphere, Tf₂O (5.68 mL, 33.6 mmol) was added. The reaction mixture was monitored by TLC (heptane:EtOAc, 1:1 and toluene:acetone, 10:1). When the reaction was complete, AcCl (2.29 mL, 32.1 mmol) was added and stirring was maintained, and the temperature was increased to room temperature. This mixture was monitored by TLC (heptane:EtOAc, 1:1 and toluene:acetone, 10:1). When the reaction was complete, it was quenched with CH₂Cl₂ and washed with 5% HCl, NaHCO₃ (saturated) and NaCl (saturated). The organic layer was dried over MgSO₄, filtered and concentrated under reduced pressure.

### Example 3

### Synthesis of phenyl 2-O-acetyl-4,6-O-benzyliden-1-thio-β-D-gulopyranoside (Structure 3 in Scheme 1)

Tetrabutylammonium nitrite (25.3 g, 87.7 mmol) was added to a solution of compound 2 (15.6 g, 29.2 mmol) in DMF (110 mL) and was kept stirring, under N₂ atmosphere, at 50° C. The reaction was observed initially to have a purple color which later was observed to be garnet colored. The reaction was monitored by TLC (heptane:EtOAc, 1:1 and toluene:acetone, 10:1) and quenched with CH₂Cl₂. The mixture was washed with 5% HCl, NaHCO₃ (saturated) and NaCl (saturated). The organic layer was dried with MgSO₄, and was filtered and concentrated under reduced pressure followed by purification by flash chromatography (eluent heptane:EtOAc, 1:1 and heptane:EtOAc, 1:2) and recrystallized from a mixture of EtOAc and heptane (1:3). ¹H NMR in CDCl₃ δ 7.60-7.57 (m, 2H, Ar), 7.43-7.40 (m, 2H, Ar), 7.37-7.34 (in, 3H, Ar), 7.29-7.25 (m, 3H, Ar), 5.50 (s, 1H, PhCH), 5.15 (d, 1H, J=10.29 Hz, H-1), 5.10 (dd, 1H, J=10.27 Hz, 2.85 Hz, H-2), 4.36 (dd, 1H, J=12.49 Hz, 1.4 Hz, H-6), 4.18 (br s, 1H, H-3), 4.08 (dd, 1H, J=3.59 Hz, 1.04 Hz, H-6), 4.03 (dd, 1H, J=12.53 Hz, 1.75 Hz, H-4), 3.88 (s, 2H, H-5+OH), 2.12 (s, 3H, OAc).

### Example 4

### Synthesis of phenyl 2-O-acetyl-4,6-O-benzylidene-1-thio-3-O-trifluoromethanesulfonyl-β-D-gulopyranoside (Structure 4 in Scheme 1)

Compound 3 (1.00 g, 2.48 mmol) was dissolved in dried CH₂Cl₂ (12.5 mL) and dried pyridine (0.40 mL, 4.96 mmol). The reaction mixture was cooled, with stirring, to −20° C. (ice and NaCl bath 3:1). Slowly and under N₂ atmosphere, Tf₂O (0.48 mL, 2.85 mmol) was added. The reaction mixture was monitored by TLC (heptane:EtOAc, 1:1 and toluene:acetone, 10:1) and when complete, was quenched with CH₂Cl₂ and washed with 5% HCl, NaHCO₃ (saturated) and NaCl (saturated). The organic layer was dried over MgSO₄, and was filtered and concentrated under reduced pressure to dryness.

### Example 5

### Synthesis of phenyl 2-O-acetyl-3-azido-4,6-O-benzylidene-3-deoxy-1-thio-β-D-galactopyranoside (Structure 5 in Scheme 1)

Tetrabutylammonium azide (2.12 g, 7.44 mmol) was added carefully to a solution of compound 4 (1.3256 g, 2.48 mmol) in DMF (10 mL) with stirring, under N₂ atmosphere, at 50° C. The reaction was monitored by TLC (E:H, 1:1) and concentrated under reduced pressure followed by purification by flash chromatography (eluent heptane:EtOAc, 2:1 and heptane:EtOAc, 1:1). ¹H NMR in CDCl₃ δ 7.61-7.58 (m, 2H, Ar), 7.44-7.41 (m, 2H, Ar), 7.39-7.36 (m, 3H, Ar), 7.30-7.24 (m, 3H, Ar), 5.59 (s, 1H, PhCH), 5.35 (t, 1H, J=9.95 Hz, H-2), 4.73 (d, 1H, J=9.63 Hz, H-1), 4.44 (dd, 1H, J=6.24 Hz, 1.60 Hz, H-6), 4.35-4.34 (dd, 1H, J=3.33 Hz, 0.88 Hz, H-4), 4.11 (dd, 1H, J=12.48 Hz, 1.67 Hz, H-6), 3.57 (d, 1H, J=1.15 Hz, H-5), 3.44 (dd, 1H, J=10.21 Hz, 3.29 Hz, H-3), 2.17 (s, 3H, OAc).

### Example 6

### Synthesis of phenyl 2-O-acetyl-3-azido-3-deoxy-1-thio-β-D-galactopyranoside (Structure 6 in Scheme 1)

Compound 5 (470 mg, 1.1 mmol) was dissolved in 80% acetic acid (75 mL) and the mixture was heated and maintained at 60° C. The reaction was monitored by TLC (heptane:EtOAc, 1:1). When the reaction was complete, the mixture was concentrated under reduced pressure with heat.

### Example 7

### Synthesis of phenyl 2,4,6-tri-O-acetyl-3-azido-3-deoxy-1-thio-β-D-galactopyranoside (Structure 7 in Scheme 1)

Acetic anhydride (30 mL) was added to a solution of compound 6 (373 mg, 1.1 mmol) in dry pyridine (30 mL). The reaction was monitored by TLC (heptane:EtOAc, 1:1) and when complete, was concentrated under reduced pressure. ¹H NMR in CDCl₃ δ 7.54-7.51 (m, 2H, Ar), 7.35-7.30 (m, 3H, Ar), 5.46 (dd, 1H, H-4), 5.23 (t, 1H, H-2), 4.73 (d, 1H, H-1), 4.15 (d, 2H, H-6, H-6), 3.94 (dt, 1H, H-5), 3.68 (dd, 1H, H-3), 2.18 (s, 3H, OAc), 2.15 (s, 3H, OAc), 2.06 (s, 3H, OAc).

### Example 8

### Synthesis of 2,4,6-tri-O-acetyl-3-azido-3-deoxy-α-D-galactopyranosyl bromide (Structure 8 in Scheme 1)

Compound 7 (237.4 mg, 560 mol) was dissolved in dry CH₂Cl₂ (2 mL), and bromine (32 μl, 620 μmol) was added. The reaction was monitored by TLC (heptane:EtOAc, 1:1). When complete, a small amount of cyclopentene was added to the reaction mixture to remove remaining untreated Br₂. The mixture was concentrated under reduced pressure and purified by quick Flash chromatography (eluent: 500 mL heptane:EtOAc, 2:1).

### Example 9

### Synthesis of 2,4,6-tri-O-acetyl-3-azido-3-deoxy-α-D-galactopyranose-1-isothiouronium bromide (Structure 9 in Scheme 1)

The sensitive bromide compound 8 (70.6 mg, 180 μmol) was immediately dissolved in dry acetonitrile (1.7 mL) and refluxed with thiourea (13.7 mg, 180 mol) under N₂ for 4 hours. The reaction was monitored by TLC (heptane:EtOAc, 1:1) and when complete, the mixture was cooled.

### Example 10

### Synthesis of bis-(2,4,6-tri-O-acetyl-3-azido-3-deoxy-β-D-galactopyranosyl)-sulfane (Structure 10 in Scheme 1)

The sensitive bromide compound 8 (77.0 mg, 196 μmol) and Et₃N (60 μl, 430 μmol) was added to the last mixture (compound 9). The reaction was monitored by TLC (heptane:EtOAc, 1:1). When the reaction was complete, the mixture was concentrated under reduced pressure without heating. The residue was purified by flash chromatography (Eluent: heptane:EtOAc, 1:1). ¹H NMR in CDCl₃ δ 5.50 (dd, 2H, H-4,), 5.23 (t, 2H, H-2, H-2′), 4.83 (d, 2H, H-1, H-1′), 4.15 (dd, 4H, H-6, H-6, H-6′, H-6′), 3.89 (dt, 2H, H-5, H-5′), 3.70 (dd, 2H, H-3, H-3′), 2.19 (s, 6H, 2OAc), 2.15 (s, 6H, 2OAc), 2.18 (s, 6H, 2OAc).

### Example 11

### Synthesis of bis-(3-azido-3-deoxy-β-D-galactopyranosyl)-sulfane (Structure 11 in Scheme 1)

Compound 10 (160 mg, 0.00024 mol) was dissolved in dry MeOH (2.6 mL) and dry CH₂Cl₂ (1.6 mL), and NaOMe (IM, 24 μL, 24 μmol) was added. The reaction was monitored by TLC (heptane:EtOAc 1:1 and D:M 5:1). When the reaction was complete, the mixture was neutralized with Duolite C436 until pH 7, and was filtered and washed with MeOH. The filtered solution was concentrated under reduced pressure. The residue was purified by flash chromatography (Eluent: CH₂Cl₂:MeOH, 5:1) to give pure compound 11 (74.1 mg, 75%). 1H NMR in CDCl₃ δ 4.72 (d, 2H, J=9.7 Hz, H-1, H-1′), 3.95 (br s, 2H, H-4, H-4′), 3.84 (t, 2H, J=9.8 Hz, H-2, H-2′), 3.74 (dd, 2H, J=11.47 Hz, 7.23 Hz, H-6, H-6′), 3.64 (dd, 2H, J=11.48 Hz, 4.72 Hz, H-6, H-6′), 3.60-3.55 (ddd, 2H, 7.15 Hz, 4.67 Hz, 0.93 Hz, H-5, H-5′), 3.36 (dd, 2H, J=10 Hz, 3.05 Hz, H-3, H-3′).

### Example 12

### Synthesis of bis-{3-deoxy-3-[4-(3-fluorophenyl)-1H-1,2,3-triazol-1-yl]-β-D-galactopyranosyl}sulfane (TD139)

TD139 was synthesized at ambient temperature by Cu(I)-catalyzed cycloaddition between bis-(3-azido-3-deoxy-β-D-galactopyranosyl)-sulfane (compound 11) and 3-fluorophenylacetylene (3 eq.) with Cu(I) (0.2 eq), triethylamine (2 eq.) in N,N-dimethylformamide (DMF, 100 mL/mmol sulfane). The reaction was monitored with TLC until complete, and concentrated and purified first by flash chromatography (Eluent: CH₂Cl₂:MeOH, 8:1), then by preparative HPLC to yield TD139 in 76% yield as a white amorphous solid. ¹H-NMR (CD₃OD, 400 MHz) d 8.59 (s, 2H, triazole-H), 7.63 (br d, 2H, 7.6 Hz, Ar-H), 7.57 (br d, 2H, 8.4 Hz, Ar-H), 7.41 (dt, 2H, 6.0 and 8.0 Hz, Ar-H), 7.05 (br dt, 2H, 2.4 and 6.4 Hz, Ar-H), 4.93 (dd, 2H, 2.4 and 10.4 Hz, H3), 4.92 (d, 2H, 10.4 Hz, H1), 4.84 (2H, 10.4 Hz, H2), 4.18 (d, 2H, 2.4 Hz, H4), 3.92 (dd, 2H, 4.2 and 7.6 Hz, H5), 3.84 (dd, 2H, 7.6 and 11.4 Hz, H6), 3.73 (dd, 2H, 4.2 and 11.4 Hz, H6); FAB-HRMS m/z calcd for C₂₈H₃₀F₂N₆NaO₈S (M+Na⁺), 671.1712. found, 671.1705.

The structure of compound TD139 is shown below:

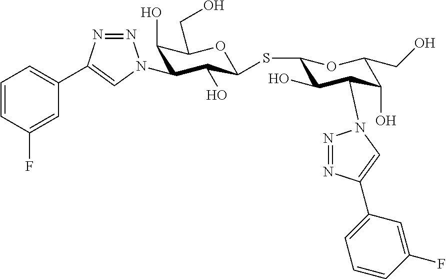

### Example 13

### Materials Used in Capillary Tubule Formation Assays

Growth Factor Reduced Matrigel (catalog number 354230) material was obtained from Becton Dickinson Company (Franklin Lakes, N.J.). Trans-well inserts having an eight micrometer (micron, μm) pore size were obtained from Corning Inc. (Corning, N.Y.; catalog number 3422). Endothelial basal medium was obtained from Lonza Inc. (Walkersville, Md.; catalog number CC-3156). Vascular endothelial growth factor (VEGF) was obtained from Cell Signal Inc. (Canton, Mass.; catalog number CRV001A).

### Example 14

### Effect of Inhibitor on Capillary Tubule Formation Assay

Matrigel was diluted to a concentration of 4.5 mg/mL, was added to an eight-chamber slide (200 μl/chamber) and was polymerized at 37° C. for 20 minutes. Human umbilical vein endothelial cells (HUVEC) were cultured in the containers and were detached by lifting from the surfaces of the container using 0.25% trypsin-ethylenediaminetetraacetic acid.

A solution was prepared containing 25 nanograms (ng)/mL VEGF in endothelial basal medium 2 (EBM-2) supplemented with 1% fetal bovine serum (FBS). HUVEC samples (20,000 cells/sample) were administered either: 25 ng/mL VEGF in EBM-2 and ten micromolar (μM) TD139 galectin-3 inhibitor (molecular weight of 600), or 25 ng/mL VEGF in EBM-2 and 50 μM of TD139.

Control HUVEC were treated with 25 ng/mL VEGF only, in EBM-2. Each of the different HUVEC samples was incubated at 37 C 5% CO₂ for six hours in the matrigel chambers.

Representative photographic images (four images per chamber) were obtained. Capillary tube formation data was obtained and was quantified by counting number of branch points. The data was normalized to control samples treated with EBM-2 medium supplemented with 1% FBS only (neither VEGF nor TD139 inhibitor added).

Confocal images were obtained using a Leica DM IRE confocal laser scanning microscope (Leica Lasertechnik, Heidelberg, Germany) equipped with a ×40/0.75 (f/1.25) objective (See Markowska et al. 2011 J of Bio Chem Vol. 286 (34): 29913-29921). Fluorescent images were acquired using an Eclipse E400 microscope (Nikon, Tokyo, Japan) with a CFI Plan Fluor ×4 (f/0.13) or CFI Plan Fluor ×20 (f/0.50) objective. The images of cell sprouts were photographed with a Nikon Eclipse TE200 phase contrast microscope with a CFI Plan Fluor ×4 and CFI Plan Fluor ×10 (f/0.30) objective. Images were acquired at room temperature with a SPOT RT color digital camera (Diagnostic Instruments, Sterling Heights, Mich.) and the SPOT Acquisition software version 4.0.6 (Diagnostic Instruments). The data were analyzed by Student's t-test (Table 1).

TABLE 1
Capillary tubule formation for human umbilical vein endothelial cells
administered VEGF and TD139 inhibitor, and control absent inhibitor

| | Fold change in tubule formation | | |
|---|---|---|---|
| | of treated cells compared to control | | |
| | cells not treated with VEGF (25 ng/mL) and inhibitor | | |
| Replicate | VEGF | VEGF + 10 μM inhibitor | VEGF + 50 μM inhibitor |
| 1 | 2.42 | 1.94 | 1.13 |
| 2 | 2.6 | 1.85 | 0.79 |
| 3 | 1.64 | 1.32 | 0.27 |
| mean | 2.22 | 1.70 | 0.73 |
| standard | 0.51 | 0.34 | 0.43 |
| deviation | | | |
| standard | 0.29 | 0.19 | 0.25 |
| error | | | |

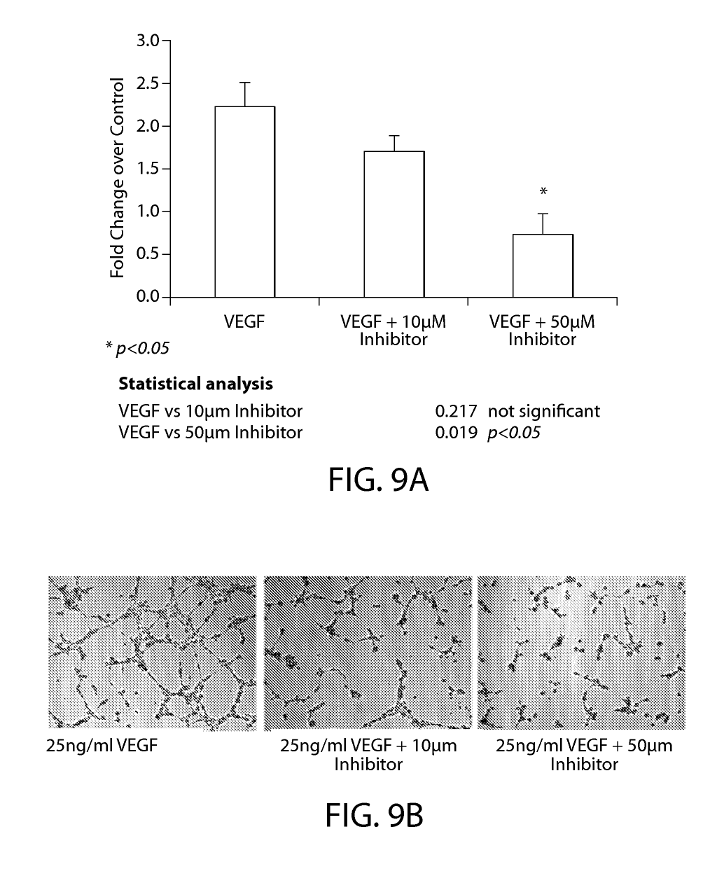

Enhanced capillary tube formation was observed in HUVEC (one- to three-fold) treated with 25 ng/mL of VEGF compared to HUVEC treated either with VEGF in EBM-2 and ten μM TD139 galectin-3 inhibitor, or with VEGF in EBM-2 and 50 μM of TD139 (Table 1, FIG. 9 A and FIG. 9B). Treatment of HUVEC with 10 μM of the TD139 inhibitor decreased of VEGF-mediated capillary tubule formation in the HUVEC, compared to HUVEC treated with VEGF only (Table 2 and FIG. 9B).

Most important, treatment of HUVEC with 50 μM TD139 inhibitor markedly reduced the extent of VEGF-mediated capillary tubule formation (n=3, p<0.05; FIG. 9B) compared to HUVEC treated with VEGF only. These data show that the galectin inhibitor substantially TD139 inhibited VEGF-induced capillary tube formation.

### Example 15

### Materials in Three Dimensional In Vitro Angiogenesis Assay/Sprouting Assay

Methyl cellulose (catalog number M0512) was obtained from Sigma Inc., (St. Louis, Mo.) Dulbecco's Modified Eagle's Medium (DMEM; catalog number 11965) was obtained from Invitrogen Inc. (Carlsbad, Calif.). Collagen type I (catalog number 354-4236) was obtained from Becton Dickinson Company. Concentrated (10×) medium 199 (catalog number 11825) was obtained from Invitrogen Inc. Concentrated (100×) penicillin-streptomycin solution (catalog number 15140) was obtained from Gibco Invitrogen Ltd. (Carlsbad, Calif.).

Polystyrene Suspension Culture Microplates (96 wells per plate; catalog number 650185) were obtained from Greiner Bio One (Frickenhausen, Germany). VEGF (catalog number 100-200) was obtained PeptoTech Inc. (Rocky Hill, N.H.). Endothelial growth medium-2 (EGM-2) (catalog number CC4176) and EBM-2 (catalog number CC3156) were obtained from Lonza Inc.

### Example 16

### Methyl Cellulose Preparation

Methyl cellulose powder (6 g) was autoclaved with stirring using a magnetic bar in a 500 mL bottle. The powder was dissolved in 250 mL of DMEM medium at 60° C. with stirring for 30 minutes, and then 250 mL DMEM at room temperature was added and the mixture was stirred for two hours at 4° C. The resulting mixture was then aliquoted into 50 mL cylindrical tubes and centrifuged (6170 revolutions per minute) for two hours at room temperature to remove debris interfering with spheroid formation. The clear supernatant was collected from the centrifuged tubes.

### Example 17

### Endothelial Cell-Spheroid Preparation

Endothelial cells (EC) were cultured in containers and were detached from the container surfaces with 0.25% trypsin. The pH of the trypsinized EC cells was neutralized with DMEM. Spheroid solution was prepared in tubes by combining 3.75 mL of methylcellulose, 1.75 mL FBS, 17.5 μl pen/strep, and 90,0000 endothelial cells, and adding DMEM to bring the total volume to 17.5 mL. The spheroid solution was mixed by inverting tubes, and 150 μl aliquots were added to each well of a 96 well plate using a multichannel pippettor and wide mouth tips. Spheroids were settled and were incubated for 24 hours at 37° C. in 5% CO₂.

### Example 18

### Collagen Preparation

Each 96 well plate yielded spheroids sufficient for four wells of a 24 well plate. The collagen solution was prepared using 2.3 mL (3.9 mg/mL) collagen type 1, 400 μl 10×M199 medium, 200 μl FBS, and 1.1 mL water. The collagen solution was neutralized with sodium hydroxide using a color pH indicator. The methyl cellulose solution (4.5 mL) was combined with 1.5 mL DMEM and warmed to 37° C. in a water bath. EC spheroids were collected using a wide mouth pipette and centrifuged (700 revolutions per minutes) for three minutes at room temperature.

The resulting supernatant was removed, and two mL of methyl cellulose solution were added to the pellet. Neutralized collagen solution (2 mL) was added to the mixture, and mixed using a pipette carefully to avoid bubble formation.

An aliquot (1 mL) of the mixed collagen solution was added to four wells of a 24 well plate, and the plate was incubated at 37 C 5% CO2 for 30 minutes to polymerize the collagen. Polymerized collagen was overlaid with 100 μl of EBM-2 and 25 ng/mL VEGF in EBM-2 supplemented either with 20 μM TD139 or 50 μM TD139. Control collagen samples were treated with 100 μl of EBM-2 and 25 ng/mL VEGF in EBM-2.

Sprouting and tubulogenesis were observed with an inverted phase-contrast microscope (Nikon) as a function of time (days), and were photographed using a SPOT RT color digital camera (Diagnostic Instruments, Sterling Heights, Mich.) and the SPOT Acquisition software version 4.0.6 (Diagnostic Instruments). See Meyer et al. 2003 J Biol Chem. 278(18): 16347-55. Representative images of the incubated samples were obtained and the cumulative length in arbitrary units of the endothelial cell sprouts was determined by ImageJ processing and analysis procedures. The data was analyzed by Student's t-test (Table 2).

Statistical analysis: control compared to presence of VEGF only is 0.0012; presence of VEGF only compared to presence of 10 μM inhibitor and VEGF is 0.119; and presence of VEGF only compared to presence of 50 μM inhibitor and VEGF is 0.079.

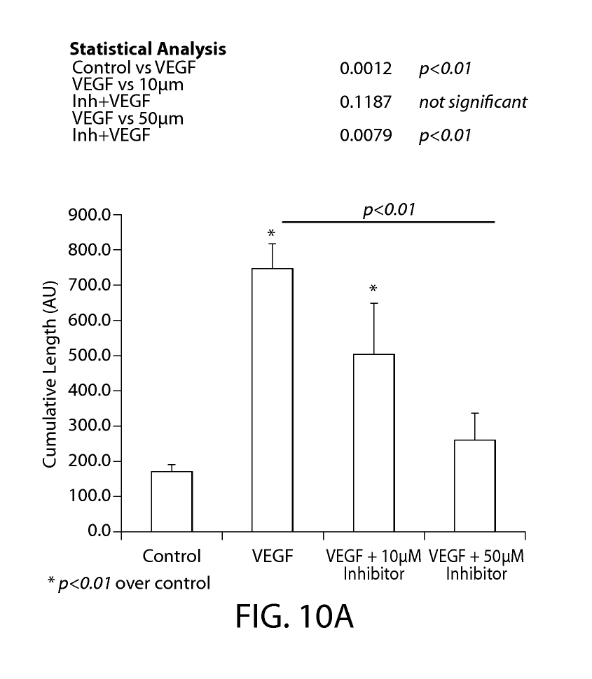

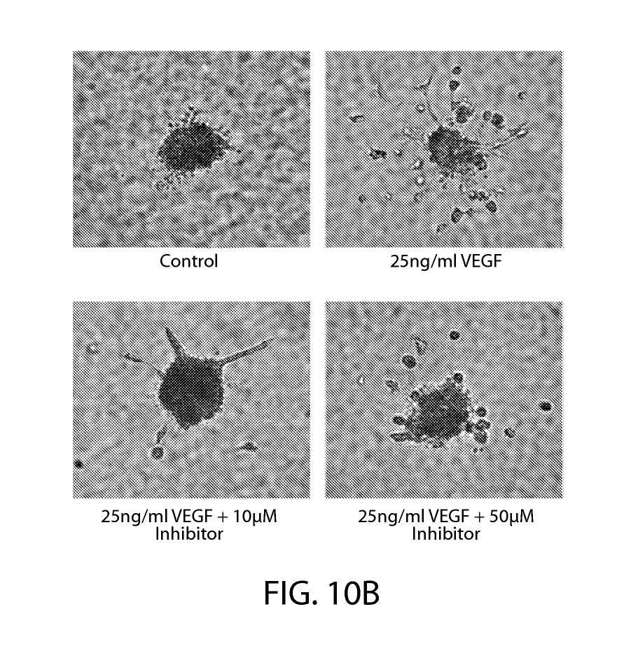

Data show that EC spheroids administered 25 ng/mL of VEGF only produced four-fold more formation of endothelial cell sprouts compared to control EC spheroid samples treated with medium only (FIG. 10A). It was observed that administering 10 μM of the TD139 inhibitor decreased sprouting by a factor of about one-third, the amount of VEGF-mediated EC sprouting compared to EC spheroids contacted with VEGF alone (Table 2 and FIG. 10B). Administering 50 μM of the TD139 inhibitor markedly further reduced the extent (about two-thirds) of VEGF-mediated sprouting (n=3, p<0.01) compared to EC spheroids treated with 10 μM of the TD139 inhibitor. Thus, a galectin inhibitor substantially inhibited an angiogenic process, viz., VEGF-induced sprouting of capillaries, and the inhibition was dose dependent with respect to the amount of inhibitor administered.

TABLE 2
In vitro angiogenesis in EC administered VEGF and TD139 inhibitor

| | Fold change in capillary sprouting of EC | | | |
|---|---|---|---|---|
| | over control cells not contracted | | | |
| | with VEGF and inhibitor | | | |
| | | | VEGF + 10 μM | VEGF + 50 μM |
| Replicate | Control | VEGF | inhibitor | inhibitor |
| 1 | 207 | 800 | 779 | — |
| 2 | N/A | 650 | 294 | 394 |
| 3 | 169 | 921 | 729 | 294 |
| 4 | 132 | 604 | 200 | 93 |
| mean | 169 | 743 | 500 | 260 |
| Standard | 38 | 145 | 296 | 153 |
| deviation | | | | |
| Standard error | 22 | 72 | 148 | 77 |

### Example 19

### Galectin Protein Inhibition Modulates VEGF Receptor Pathway In Vivo

Vascular endothelial growth factor (VEGF) signaling though VEGF receptor-2 (VEGFR2) is the primary angiogenic pathway, of which galectin-1 and galectin-3 proteins are important modulators. TD139 has a high-affinity for galectin-1 (equilibrium dissociation constant or K_{D} of 12 nM) and galectin-3 (K_{D}=14 nM). See Lepur A et al. 2012 Biochim Biophys Acta. 1820(7): 804-18, which is incorporated by reference herein in its entirety. Compound TD139 specifically binds each of galectin-1 and galectin-3.

Examples herein show that TD139 targeting both galectin-1 and galectin-3 affected VEGF-A/VEGFR2 signaling. Efficacy of TD139 to inhibit pathological angiogenesis is shown in Examples herein. Without being limited by any particular theory or mechanism of action, it is here envisioned that TD139 treatment interfered with VEGF-A/VEGFR2 signaling and abrogated VEGF-A-induced angiogenesis.

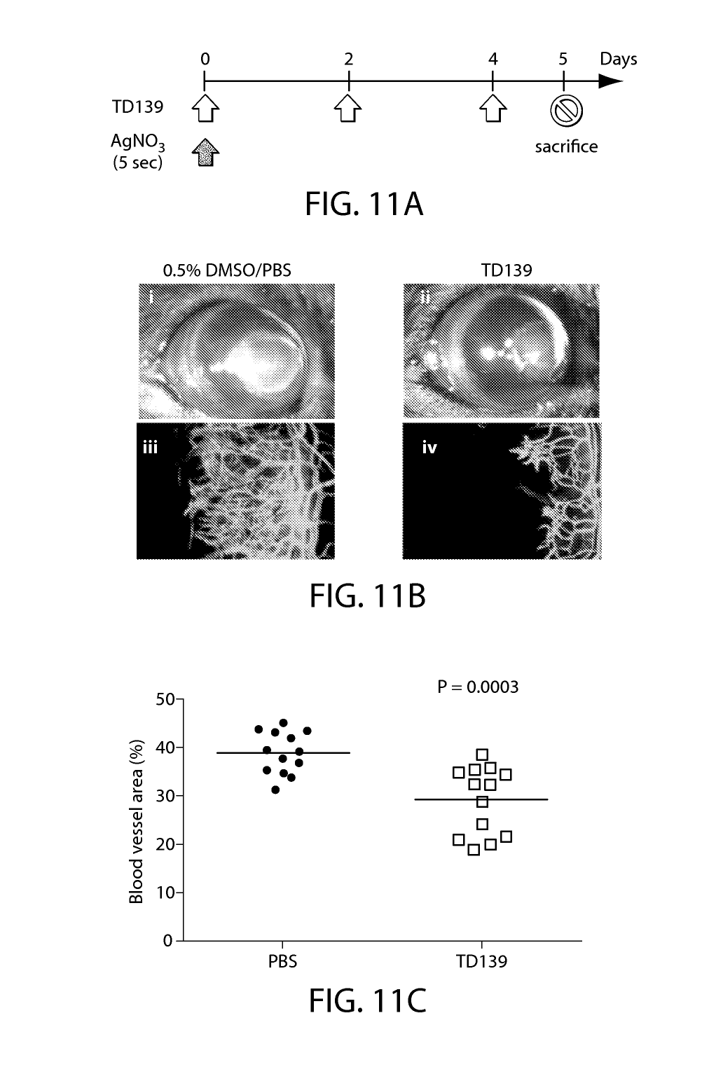

Neovascularization in the eye was induced in mouse corneas by cauterization using silver nitrate. FIG. 11A shows the regimen/administration schedule for the C57B/6L murine subjects. A group of subjects were sub-conjunctivally injected every other day with 10 μL of TD139 (325 ng) in PBS containing 0.5% DMSO. Control subjects were sub-conjunctivally injected with vehicle only (PBS containing 0.5% DMSO only). Another group of subjects were administered eye drops of either 10 μl of vehicle alone or 50 μM TD139 in vehicle once per day.

After five days of either sub-conjunctival injection treatment or eye drop treatment, subjects were sacrificed, and flat mounts of corneas were excised, photographed, and stained with anti-CD31 to visualize blood vessels (FIG. 11B, magnification is ×100). The density of blood vessels covering the whole cornea was quantified by ImageJ and was analyzed with Student's t test (FIG. 11C).

Representative photographs of eyes from the subjects five days after cauterization are shown in FIG. 11B top row. Eyes of control subjects treated with PBS show strong indicia of angiogenesis and formation of new blood vessels (FIG. 11B left column top row). Most important, analysis of photographs of TD139-treated eyes show reduced angiogenesis compared to the control subjects (FIG. 11B right column top row).

Representative segments from corneal flat mounts stained with anti-CD31 are shown in FIG. 11B bottom row. Eyes from control subjects treated with PBS alone show intense CD31 staining (FIG. 11B left column bottom row). Most important, corneas from subjects treated with TD139-treated cornea showed reduced CD31 staining compared to eyes from control subjects treated with PBS only.

Further, the density of blood vessels covering the cornea from control subjects treated with PBS only was about 40%. The density of blood vessels covering the cornea from subjects treated with TD139 was significantly reduced (28%). See FIG. 11C. It was observed that both subconjunctival injection and topical application of TD139 eye drops reduced cautery-induced corneal neovascularization in vivo. Thus TD139 was observed to be an effective in vivo inhibitor of corneal neovascularization, which is an important system of ocular angiogenesis.

### Example 20

### Galectin-1 and Galectin-3 Proteins Bind to and Interact with VEGFR-2

Lysates from HUVEC expressing VEGFR-2 were incubated with galectin-1 protein conjugated agarose beads or galectin-3 protein conjugated agarose beads in the presence of 0.1 molar (M) sucrose or 0.1 M lactose. Control HUVEC lysates were incubated with galectin-1 agarose beads or galectin-3 agarose beads only. The beads were rinsed to remove unbound material.

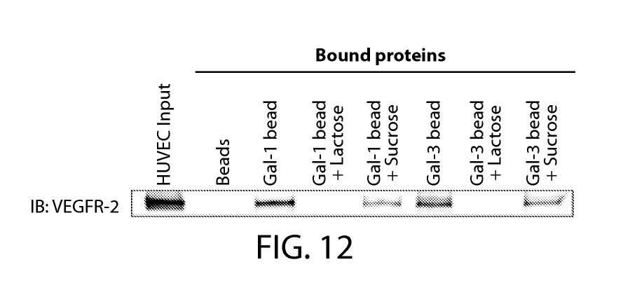

HUVEC lysate material bound to the beads was eluted with the Laemmli sample buffer and was analyzed using gel electrophoresis (4-20% SDS-PAGE gels). Following blocking, membrane blots were probed using anti-VEGFR-2 antibody (FIG. 12). Data show that the lysates from the HUVEC expressing VEGFR-2 bound specifically to each of galectin-1 protein conjugated agarose beads and to galectin-3 protein conjugated agarose beads. Binding of the HUVEC lysate material to each of the galectin-1 and galectin-3 protein beads was observed to be inhibited by treatment with lactose, a competing saccharide. Further, the VEGFR2 HUVEC lysate binding to the beads was not observed for beads treated with sucrose, a noncompeting saccharide.

Without being limited by any particular theory or mechanism of action, it is here envisioned that interaction between VEGFR-2 and galectin-1 protein and between VEGFR-2 and galectin-3 protein is carbohydrate-dependent. The carbohydrate binding domain of galectin-1 and galectin-3 specifically bound and interacted with VEGFR2.

### Example 21

### TD139 Inhibited VEGF-A Induced Endothelial Cell Sprouting

HUVEC spheroids were prepared as described in examples herein, and the spheroids were seeded into type I collagen gels. After six hours of incubations, the gels were treated with either: VEGF-A (100 ng/ml) only, VEGF-A (100 ng/ml) and TD139 (0.01 μM, 0.1 μM, 1 μM, 5 μM, or 10 μM), or TD139 (10 μM) only. Control spheroids were treated with neither VEGF-A nor TD139. After 24 hours of treatment, spheroids were stained with calcein AM, a cell-permeant dye that indicates cell viability in eukaryotic cells (Invitrogen Inc.), and the spheroids were photographed by a fluorescent microscope. Cumulative sprout lengths were quantified by ImageJ.

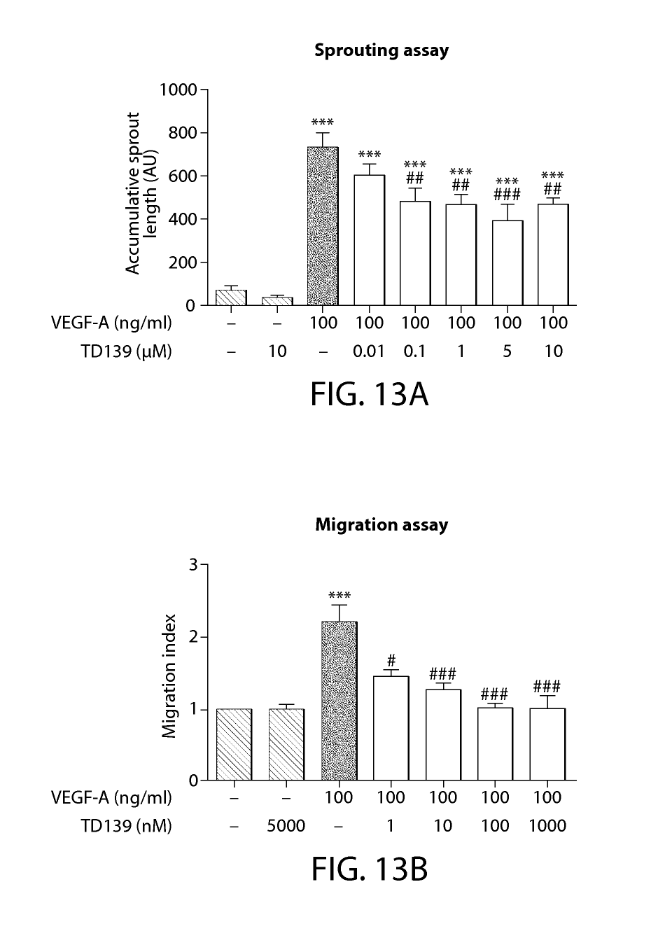

HUVEC spheroids treated with VEGF-A only were observed to have increased HUVEC sprouting compared to control cells not treated with VEGF (FIG. 13A). Most important, HUVEC spheroids treated with TD139 were observed to have reduced lengths of sprouting compared to HUVEC spheroids treated with VEGF-A only (FIG. 13A). Treatment with each of 0.1 μM, 1 μM, 5 μM, and 10 μM TD139 significantly inhibited VEGF-A-induced sprouting of HUVEC.

### Example 22

### TD139 Inhibited VEGF-A-Induced Endothelial Cell Migration

HUVEC were incubated without serum overnight, detached with Accutase, re-suspended in 1% FBS/M199 and added in an upper chamber separated from a bottom chamber by a membrane. The bottom chamber was filled with VEGF-A in the presence of different concentrations (1 nM, 10 nM, 100 nM, 1000 nm, or 5000 nM) of TD139 in 1% FBS/M199, or a control without TD139. Control HUVEC were treated with neither VEGF-A nor TD139.

After three hours of incubation, HUVEC were observed to have migrated to the lower side of the membrane and were counted using high-power field (HPF) analysis (FIG. 13B). Data were plotted as mean±SEM and analyzed with one-way ANOVA. *P<0.05 vs control; ***P<0.001 vs control; ^{###}P<0.001 vs VEGF-A.

VEGF-A was observed to have caused increased chemotaxis of HUVEC compared to control HUVEC treated with neither VEGF nor with TD139. TD139 inhibited VEGF-A induced chemotaxis in HUVEC compared to HUVEC treated with VEGF only. It was observed using a three-dimensional sprouting assay that TD139 significantly abrogated VEGF-A-induced chemotaxis and migration in a dose-dependent manner.

### Example 23

### TD139 is Nontoxic to HUVEC

HUVECs were incubated in 1% FBS/M199 overnight and then treated for three hours with either 0.1% DMSO, 0.1% Triton™ X-100 (positive control for killing), or TD139 (5 μM). Control HUVEC were treated with vehicle containing none of DMSO, Triton™ X-100, or TD139.

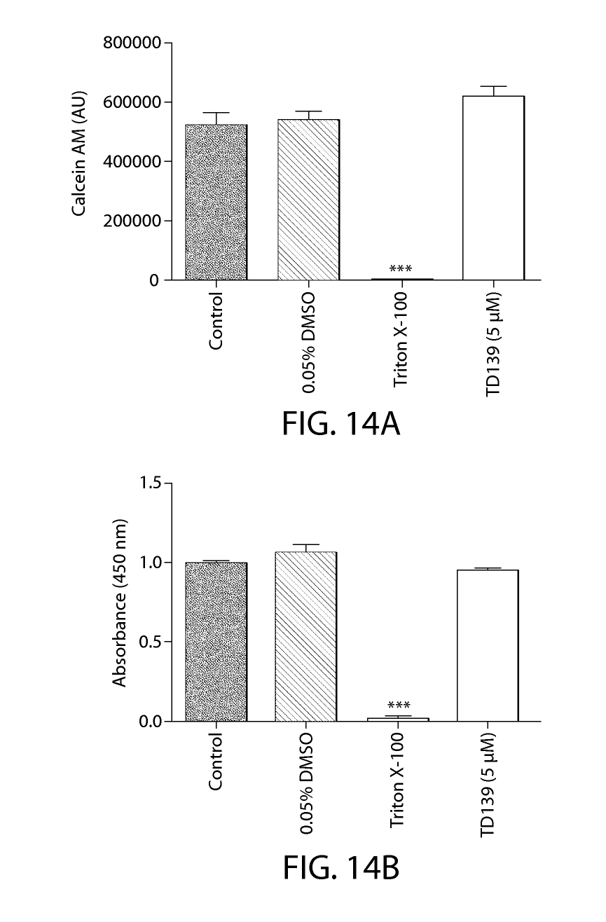

The cell viabilities of the treated HUVEC and control HUVEC were determined with each of calcein AM and WST-1, two systems used to determine cell viability. Calcein AM was added to a group of treated HUVEC, which were incubated for 30 minutes (FIG. 14A). Another group of treated HUVEC were incubated for two hours with WST-1, a tetrazolium salt used for determining cell proliferation and is an indicator or cell viability (FIG. 14B). Data are plotted with mean±SEM and analyzed with one-way ANOVA. ***P<0.001 vs control.

Fluorescence emission was detected by a spectrophotometer for each treated group contacted with either calcein AM or WST-1. Both calcein AM or WST-1 systems show similar results, specifically data show that cell viability was reduced for HUVEC treated with Triton™ X-100 compared to control untreated HUVEC. HUVEC viability was not reduced after either TD139 treatment or DMSO treatment compared to control HUVEC (FIG. 14B). Thus, the inhibitory effects of TD139 on VEGF-A-induced sprouting and migration were not a result of alteration of cell viability as assessed by two different reagents, Calcien AM and WST-1.

### Example 24

### VEGFR2 Expression is not Affected by TD139 Treatment

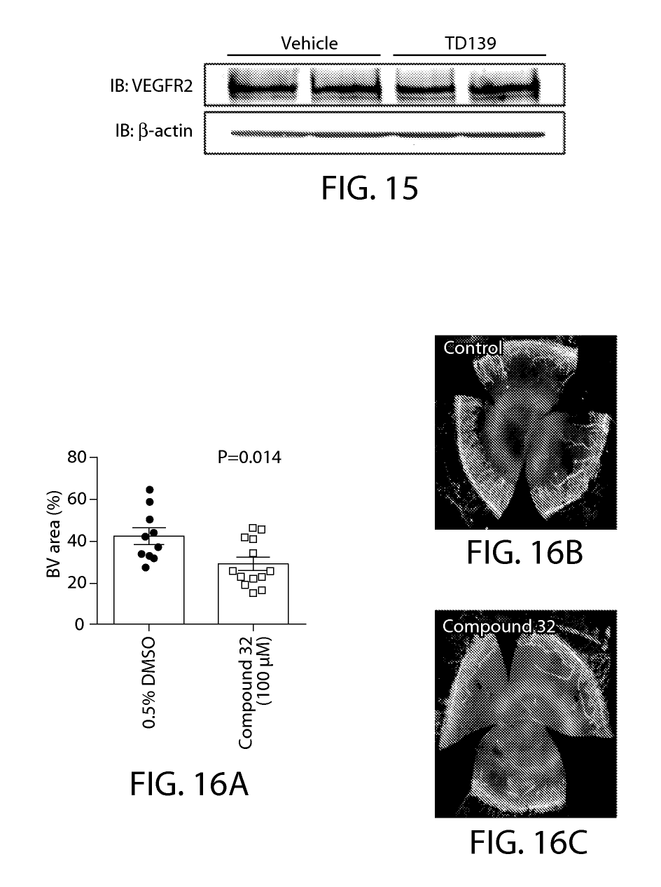

HUVEC were incubated in 1% FBS/M199 overnight and then treated with PBS containing 0.1% DMSO and 10 μM TD139 for six hours. Control HUVEC were not treated with TD139. Cell lysates were analyzed using gel electrophoresis (4-20% SDS-PAGE gels), and the membrane blots were probed with either anti-VEGFR-2 antibodies or β-actin antibodies. Data show that VEGFR2 expression levels and β-actin expression levels were similar in each of control HUVEC and TD139-treated HUVEC. It was observed that TD139 treatment of HUVEC had little or no effect on total protein expression of VEGFR2 (FIG. 15).

Examples herein show that TD139 targeted galectin-1 protein and galectin-3 protein, and that TD139 ameliorated pathological angiogenesis in cells in culture, and in tissues and in subjects in vivo. Inhibition of galectin-1 and galectin-3 using the galactoside compounds described herein was shown to be an effective treatment and preventive regimen for ocular angiogenesis and for a variety of disease conditions related to ocular angiogenesis including age-related macular degeneration and corneal neovascularization.

### Example 25

### Delivery of Galectin Inhibitors to Prevent Ocular Angiogenesis

Alternative galectin inhibitors and routes of administering the compounds are investigated for treating or preventing ocular angiogenesis.

The inhibitors specific for galectin proteins (galectin-1, galectin-3, galectin-7 and galectin-8) or nucleotide sequences that encode the galectin proteins are prepared. The inhibitors included antibodies, small galactoside compounds, siRNA, and vectors encoding a protein modulator of galectin activity. The subjects are adminstered the inhibitor by a route of administration selected from: intravenous, intramuscular, intraperitoneal, intradermal, intrapulmonary, intravaginal, oral, buccal, topical, sublingual, intranasal, topical, and intraocular. Control subjects are adminstered phosphate buffered saline or empty vectors not encoding the modulator. Animals are induced to have ocular angiogenesis and/or neovascularization using cauterization as described herein. Animals are sacrificed and tissue samples are removed and analyzed for indicia of angiogenesis including endothelial cell sprouting.

Administering the galectin inhibitors is observed to result in prevention of ocular angiogenesis in the subjects compared to results for control subjects. Tissues from the galectin inhibitor treated subjects showed reduced level of ocular angiogenesis compared to tissues from control subjects with each type of inhibitor used (i.e., antibody, galactoside, siRNA, and vector), and route of administration.

### Example 26

### Delivery of Galectin Inhibitors to Prevent Ocular Fibrosis

Ocular diseases of the eye cause catastrophic loss of vision as a result of abnormal angiogenesis, wound healing, tissue ischemia, or inflammation (Friedlander, M. 2007 J Clin Invest. 117(3): 576-586, which is incorporated by reference herein in its entirety). Fibrosis is often caused by physical wounds or metabolic malfunctions, including responses to inflammation, ischemia, and degenerative diseases. Ocular fibrosis involves infiltration into the eye and ocualar tissues (e.g., retina) of inflammatory cells, fibroblasts, or fibroblast-like cells, and also involves conditions such as neovascularization and altered vascular permeability.

Animal models and in vitro models for ocular fibrosis use a number of different systems including basic solutions (e.g., sodium hydroxide), proteins (e.g., dispase), and cells such as macrophage-rich peritoneal exudate cells. See Okada et al. 2001 American Journal of Pathology 178(6): 2654-2664; Tan et al. 2012 Molecular Vision 18: 887-900; and Zhang et al. 2012 International Journal of Ophthalmology 5(3): 307-311, each of which is incorporated by reference herein in its entirety.

A model of ocular fibrosis using a sodium hydroxide solution applied to eyes of subjects (Okada et al. 2001 American Journal of Pathology 178(6): 2654-2664) is used herein to test the ability of the pharmaceutical compositions described herein to inhibit, prevent, or modulate ocular fibrosis. The inhibitors specific for galectin proteins (galectin-1, galectin-3, galectin-7 and galectin-8) or nucleotide sequences that encode the galectin proteins are prepared. The inhibitors included antibodies, small galactoside compounds, siRNA, and vectors encoding a protein modulator of galectin activity. The subjects are adminstered the inhibitor by a route of administration selected from: intravenous, intramuscular, intraperitoneal, intradermal, intrapulmonary, intravaginal, oral, buccal, topical, sublingual, intranasal, topical, and intraocular. Control subjects are adminstered phosphate buffered saline or empty vectors not encoding the modulator. Animals are induced to have ocular fibrosis as described herein. Animals are sacrificed and tissue samples are removed and analyzed for indicia of ocular fibrosis including proliferation of fibroblasts.

Administering the galectin inhibitors is observed to result in prevention of ocular fibrosis in the subjects compared to results for control subjects. Tissues from the galectin inhibitor treated subjects showed reduced level of ocular fibrosis compared to tissues from control subjects with each type of inhibitor used (i.e., antibody, galactoside, siRNA, and vector), and route of administration.

### Example 27

### Galectin-3 Specific Inhibitor Reduced Corneal Neovascularization

Neovascularization in corneas which may occur from trauma, surgery, or disease causes pain, blurred vision, tearing, redness, and extreme sensitivity to light. To determine whether an inhibitor with high specific affinity for galectin-3 reduces neovascularization in eyes of subjects, an animal model of truma was used in which corneas of normal subjects, containing little or no blood vessels, were subjected to trauma to induce neovascularization in the cornea affecting the function of the eye.

Compounds were synthesized and tested whether the compounds bind specifically to galectin 1 and galectin-3. Methods for determining binding of molecules to galections are described below. See also Brevetto et al., U.S. patent publication number 2004/0147730 published Jul. 29, 2004. Microtiter plate wells were coated with recombinant galectin-1 or galectin-3 (10 μg/ml, 50 μl/well), and then washed with wash solutions containing PBS and detergent. The wells were blocked with BSA, and washed with wash solution/buffer. An amount of either Compound 32 (described further below), TD139 (describe above), or a non-specific lectin was prepared and added (100 μL/well) in duplicate to wells. Control wells included PBS only. An amount of galectin receptor-HRP conjugate (100 μL/well, 1 mg/mL) specific for either galectin-1 or galectin-3 was added to the respective wells. The wells were incubated for one hour and then washed with buffer at room temperature. The horse-radish peroxidase enzyme reaction was performed using a TMB-peroxidase substrate kit (BioRad Inc.). The reaction was stopped after one hour by addition of 1N sulfuric acid (100 μL/well) and the optical density was read at 450 nm. Data show that Compound 32 specifically bound to galectin-3 and not to galectin-1, and that TD139 specifically bound galectin-1 and galectin-3. The non-specific lectin did not bind to either galectin-1 or galectin-3.

Compound 32 is identified in, and was prepared by method described in Nilsson, et al., PCT application number PCT/EP2013/051339, international publication number WO 2013/110704 A1 published Aug. 1, 2013, the contents of which are hereby incorporated herein by reference.

The corneas of eight week-old C57BL/6 mice were cauterized with silver nitrate for five seconds. Test subjects were administered ten μl of Compound 32 (100 μM in 0.5% DMSO) subconjunctivally injected into corneas at days 0, 2, 4 and 6. The cauterized corneas of control subjects were subconjunctivally injected with DMSO vehicle only (0.5% DMSO in PBS), and the test subjects and control subjects respectively were administered daily eye drops (five μl) of either Compound 32 or control DMSO vehicle respectively. At day 7, corneas were excised and analyzed.

Subjects subconjunctivally injected with Compound 32 galectin-3 inhibitor showed significantly lower percent area of blood vessels compared to control subjects injected with the vehicle only (29% for Compound 32 injected subjects; 42% for control subjects). Combined data from two independent results are shown in FIG. 16A. Representative fluorescent imaging of corneas from cauterized eyes of subjects subconjunctivally injected with either control or Compound 32 are shown in FIGS. 16B and 16C. Significantly fewer blood vessels and reduced amount of neovascularization were observed in corneas from subjects subconjunctivally injected with Compound 32 compared to corneas from control subjects injected with the DMSO and PBS control vehicle. Data in FIG. 16A are plotted as Mean±SEM and analyzed using Student's t test.

### Example 28

### Model System for Inducing Corneal Fibrosis and Retinal Gliosis

A mouse model system was used to determine efficacy of galectin-3 inhibitor TD139 to reduce corneal fibrosis and retinal gliosis (see Paranthan, R. R. et al. 2011 Molecular Vision 17:1901-1908). C57BJ/6 mice were anesthetized by intraperiteoneal injection of ketamine and xylazine mixture. Corneas were topically anesthetized by application of proparacain eye drop. A drop (1.5 μl) of dilute 0.15M sodium hydroxide was applied for one minute to the central cornea of each mouse. The cornea was immediately washed extensively with PBS, and corneal and limbal epithelium was gently removed with an ALGEBRUSH™ optical device (Storz Ophthalmic Instruments, St. Louis, Mo.). An antibiotic ointment was applied to cover each cornea. An amount (10 μl) of TD139 (50 mM) or PBS vehicle was injected into the subconjunctival space each day for 14 consecutive days after alkali treatment/surgery.

Opacity of the cornea was scored on day 7 and 14 after alkali treatment using the following criteria: 0 indicates clear; 1 indicates opacity area/size is less than that of the pupil; 2 indicates that opacity area is bigger than pupil; 3 indicates that opacity area is larger than 2/3 area of the cornea; 4 indicates that whole cornea is opaque. Subjects were sacrificed on day fourteen after alkalisurgery using a mixture of ketamine and xylazine. Corneas and retinas were dissected and lysed in ice-cold lysis buffer containing 5 mM NaF, 1 mM PMSF, 1 mM DTT, 20 mM HEPES, 1 mM EDTA, 400 mM NaCl, 1 mM EGTA, 0.1% NP-40 and protease inhibitor cocktail (Roche). Aliquots of corneal protein (20 μg) and of retinal protein (30 μg) were loaded to each lane of a gel and transferred onto nitrocellulose membranes. Blots were probed overnight at 4° C. with rabbit antibody specific mouse α-SMA (1:10,000; Abcam) or rabbit antibody specific for GFAP (marker for intermediate filament found in retinal gliosis; 1:50,000; Abcam). The membranes were washed and incubated with goat anti rabbit IRDye 800CW antibody (1:10,000; Li-Cor Inc.), the membranes were scanned and quantified with Odessey imaging system (Li-Cor Inc.).

### Example 29

### TD139 Inhibited Corneal Fibrosis

SMA (smooth muscle actin) is a marker of corneal fibrosis. Eyes were treated with alkali as described in Example 28 to use in a model system to determine whether TD139 inhibits corneal fibrosis. Subject eyes were injected subconjunctively with 10 μl of 50 mM TD139 for 14 consecutive days after alkali treatment. Control subjects were injected subconjunctively with control PBS vehicle only. Opacity of the eye was scored on day 7 and day 14. Corneas were collected and lysed in lysis buffer. FIG. 17A is a set of representative images of eyes seven days after alkali treatment and subconjunctival injection TD139.

Quantification of corneal opacity of subjects treated with TD139 was performed seven days after alkali treatment using biomicroscopy and an opacity scale in which: 0 indicates clear eyes with no opacity; 1 indicates opacity area less than pupil region/area; 2 indicates opacity area larger than pupil region, 3 indicates opacity area larger than two-thirds of cornea region, and 4 indicates opaque over entire cornea region. (n=9 mice/group). P<0.05. FIG. 17B.

It was observed that injection of TD139 reduced corneal opacity in the eye compared to injection with control PBS vehicle. Western blot analysis of corneal cell lysates shows that α-SMA expression was reduced 30% by injection of TD139 compared to control subjects injected with PBS. FIG. 17C.

### Example 30

### TD139 Inhibits Retinal Gliosis

Glial fibrillary acidic protein (GFAP) is a marker for intermediate filaments indicative of retinal gliosis. Murine eyes were treated with alkali as described in Example 28 to determine whether TD139 would inhibit retinal gliosis. An amount (10 μl) of 50 mM TD139 was injected subconjunctively for 14 consecutive days after alkali treatment. Control subjects were injected subconjunctively with control PBS vehicle only. Subjects were sacrificed and retinas were collected and lysed in lysis buffer.

It was observed by western blots of retinal cell lysates that TD139 reduced expression of GFAP compared to control subjects administered PBS. FIG. 18. TD139 administration reduced retinal gliosis (FIG. 18) in eyes, and reduced corneal fibrosis (FIG. 17A-C).

As described above, compositions containing galectin inhibitors are shown herein to be effective for use in treating ocular disorders and diseases, including fibrosis and gliosis of the cornea and retina, and are effective to inhibit fibrosis as well as neovascularization and vessel proliferation in damaged or diseased tissue. Because galectins are widely distributed in different tissues, the use of specific pyranose-based inhibitors to selectively target and inhibit one or more specific galectins offers the prospect of minimizing undesired collateral interactions and providing a general ocular treatment, while the localized ocular environment will allow detailed histological observations of several angiogenic and tissue growth processes predictive of therapeutic treatments by modulation or inhibition of galectins applied to tumors and diseases in other body tissues.

1. A pharmaceutical composition for use in a method of treatment for at least one selected from: ocular angiogenesis, and ocular fibrosis, wherein the composition comprises a pharmaceutically suitable carrier or a diluent and an amount of the composition effective to inhibit or to modulate an activity of a galectin protein or portion thereof sufficient to inhibit the at least one from ocular angiogenesis or ocular fibrosis.

2. The composition according to claim 1, wherein the composition is further characterized by at least one of:
being selected from: a drug, a polymer, a protein, a peptide, a carbohydrate, a low molecular weight compound, an oligonucleotide, a polynucleotide, and a genetic material such as DNA or RNA; for example, the drug is a beta-galactoside, which is derivatized or functionalized; and
the galectin protein is selected from the group of galectin-1, galectin-3, galectin-7, and galectin-8; and
the composition is effective in a method to treat or prevent a disease or a condition associated with the ocular angiogenesis, for example the disease or the condition is associated with excessive neovascularization, for example, the disease or condition is selected from the group of: a surgery associated with scarring for example a glaucoma filtration surgery; neovascular glaucoma: a corneal injury; post-conjunctivitis scarring: pterygium, age related-macular degeneration (AMD) for example wet AMD or dry AMD; conversion from dry to wet AMD; proliferative diabetic retinopathy; diabetic macular adema; and corneal neovascularization (trachoma); and
the composition further comprises at least one agent selected from the group consisting of: anti-angiogenic, anti-tumor, antiviral, antibacterial, anti-mycobacterial, anti-fungal, anti-proliferative and anti-apoptotic.

3-6. (canceled)

7. The composition according to claim 1 having the following general formula: 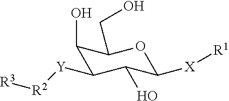
wherein the configuration of the pyranose ring is D-galacto;
X is selected from the group consisting of O, S, NH, CH.sub.2, and NR⁴, or is a bond;
Y is selected from the group consisting of NH, CH₂, and NR⁴, or is a bond;
R¹ is selected from the group consisting of: a saccharide; hydrogen, an alkyl group, an alkenyl group, an aryl group, a heteroaryl group, and a heterocycle;
R² is selected from the group consisting of CO, SO₂, SO, PO, and PO₂;
R³ is selected from the group consisting of: an alkyl group of at least 4 carbon atoms, an alkenyl group of at least 4 carbon atoms, an alkyl or alkenyl group of at least 4 carbon atoms substituted with a carboxy group, an alkyl group of at least 4 carbon atoms substituted with both a carboxy group and an amino group, and an alkyl group of at least 4 carbon atoms substituted with a halogen; a phenyl group, a phenyl group substituted with a carboxy group, a phenyl group substituted with at least one halogen, a phenyl group substituted with an alkoxy group, a phenyl group substituted with at least one halogen and at least one carboxy group, a phenyl group substituted with at least one halogen and at least one alkoxy group, a phenyl group substituted with a nitro group, a phenyl group substituted with a sulfo group, a phenyl group substituted with an amine group, a phenyl group substituted with a hydroxy group, a phenyl group substituted with a carbonyl group and a phenyl group substituted with a substituted carbonyl group; and a phenyl amino group;
R⁴ is selected from the group consisting of hydrogen, an alkyl group, an alkenyl group, an aryl group, a heteroaryl group, and a heterocycle.

8-12. (canceled)

13. The composition according to claim 7, wherein the composition is methyl 2-acetamido-2-deoxy-4-O-(3-[3-carboxypropanamido]-3-deoxy-β-D-galactopyranosyl)-β-D-glucopyranoside; methyl 2-acetamido-2-deoxy-4-O-(3-[{Z}-3-carboxypropenamido]-3-deoxy-β-D-galactopyranosyl)-β-D-glucopyranoside; methyl 2-acetamido-2-deoxy-4-O-(3-benzamido-3-deoxy-β-D-galactopyranosyl)-β-D-glucopyranoside; methyl 2-acetamido-2-deoxy-4-O-(3-[2-carboxy-benzamido]-3-deoxy-β-D-galactopyranosyl)-β-D-glucopyranoside; methyl 2-acetamido-2-deoxy-4-O-(3-[4-methoxy-2,3,5,6-tetrafluorbenz-amido]-3-deoxy-β-D-galactopyranosyl)-β-D-glucopyranoside; methyl 2-acetamido-2-deoxy-4-O-(3-[2-carboxy-3,4,5,6-tetrafluorbenzamido]-3-deoxy-β-D-galactopyranosyl)-β-D-glucopyranoside; methyl 2-acetamido-2-deoxy-4-O-(3-methanesulfonamido-3-deoxy-β-D-galactopyranosyl)-β-D-glucopyranoside; methyl 2-acetamido-2-deoxy-4-O-(3-[-4-nitrobenzenesulfonamido]-3-deoxy-β-D-galactopyranosyl)-β-D-glucopyranoside, methyl 2-acetamido-2-deoxy-4-O-(3-phenylaminocarbonylam-ino-3-deoxy-β-D-galactopyranosyl)-β-D-glucopyranoside; methyl 2-acetamido-2-deoxy-4-O-(3-aminoacetamido-3-deoxy-β-D-galactopyranosyl)-β-D-glucopyranoside; methyl 2-acetamido-2-deoxy-4-O-(-3-[{2S}-2-amino-3-carboxy-propanamido]-3-deoxy-β-D-galactopyranosyl)-β-D-glucopyranoside.

14. The composition according to claim 1 having the general formula: 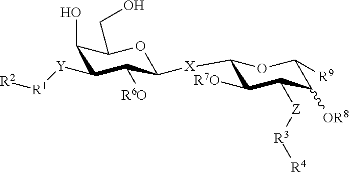
wherein the configuration of at least one of the pyranose rings is β-D-galacto;
X is selected from the group consisting of O, S, SO, SO₂, NH, CH₂, and NR⁵,
Y is selected from the group consisting of O, S, NH, CH₂, and NR⁵, or is a bond;
Z is selected from the group consisting of O, S, NH, CH₂, and NR⁵, or is a bond;
R¹ and R³ are independently selected from the group consisting of CO, SO₂, SO, PO₂, PO, and CH₂ or is a bond;
R² and R⁴ are independently selected from the group consisting of: an alkyl group of at least 4 carbons, an alkenyl group of at least 4 carbons, an alkyl group of at least 4 carbons substituted with a carboxy group, an alkenyl group of at least 4 carbons substituted with a carboxy group, an alkyl group of at least 4 carbons substituted with an amino group, an alkenyl group of at least 4 carbons substituted with an amino group, an alkyl group of at least 4 carbons substituted with both an amino and a carboxy group, an alkenyl group of at least 4 carbons substituted with both an amino and a carboxy group, and an alkyl group substituted with one or more halogens; a phenyl group substituted with at least one carboxy group, a phenyl group substituted with at least one halogen, a phenyl group substituted with at least one alkoxy group, a phenyl group substituted with at least one nitro group, a phenyl group substituted with at least one sulfo group, a phenyl group substituted with at least one amino group, a phenyl group substituted with at least one alkylamino group, a phenyl group substituted with at least one arylamino group, a phenyl group substituted with at least one dialkylamnino group, a phenyl group substituted with at least one hydroxy group, a phenyl group substituted with at least one carbonyl group and a phenyl group substituted with at least one substituted carbonyl group; or a naphthyl group, a naphthyl group substituted with at least one carboxy group, a naphthyl group substituted with at least one halogen, a naphthyl group substituted with at least one alkoxy group, a naphthyl group substituted with at least one nitro group, a naphthyl group substituted with at least one sulfo group, a naphthyl group substituted with at least one amino group, a naphthyl group substituted with at least one alkylamino group, a naphthyl group substituted with at least one arylamino group, a naphthyl group substituted with at least one dialkylamnino group, a naphthyl group substituted with at least one hydroxy group, a naphthyl group substituted with at least one carbonyl group and a naphthyl group substituted with at least one substituted carbonyl group; a heteroaryl group, a heteroaryl group substituted with at least one carboxy group, a heteroaryl group substituted with at least one halogen, a heteroaryl group substituted with at least one alkoxy group, a heteroaryl group substituted with at least one nitro group, a heteroaryl group substituted with at least one sulfo group, a heteroaryl group substituted with at least one amino group, a heteroaryl group substituted with at least one alkylamino group, a heteroaryl group substituted with at least one dialkylamino group, a heteroaryl group substituted with at least one arylamino group, a heteroaryl group substituted with at least one hydroxy group, a heteroaryl group substituted with at least one carbonyl group and a heteroaryl group substituted with at least one substituted carbonyl group; R⁶ and R⁸ are independently selected from the group consisting of a hydrogen, an acyl group, an alkyl group, a benzyl group, and a saccharide; R⁷ is selected from the group consisting of a hydrogen, an acyl group, an alkyl group, and a benzyl group; R⁹ is selected from the group consisting of a hydrogen, a methyl group. hydroxymethyl group, an acyloxymethyl group, an alkoxymethyl group, and a benzyloxymethyl group.

15-20. (canceled)

21. The composition according to claim 14, wherein the composition is bis-(3-deoxy-3-benzamido-β-D-galactopyranosyl)sulfane (17), bis-(3-deoxy-3-(3-methoxybenzamido)-β-D-galactopyranosyl)sulfane; bis-(3-deoxy-(3,5-dimethoxybenzamido)-β-D-galactopyranosyl)sulfane; bis-(3-deoxy-3-(4-nitrobenzamido)-β-D-galactopyranosyl)sulfane; bis(3-deoxy-3-(2-naphthamido)-β-D-galactopyranosyl)sulfane; bis-(3-deoxy-3-(4-methoxybenzamido)-β-D-galactopyranosyl)sulfane; bis-(3-deoxy-3-(3-nitrobenzamido)-β-D-galactopyranosyl)sulfane; bis-(3-deoxy-3-[4-(dimethylamino)-benzamido]-β-D-galactopyranosyl)sulfane; bis-(3-deoxy-3-(4-methylbenzamido)-β-D-galactopyranosyl)sulfane; bis-(3-deoxy-3-(4-chlorobenzamido)-β-D-galactopyranosyl)sulfane; bis-(3-deoxy-3-(4-tert-butylbenzamido)-β-D-galactopyranosyl)sulfane; bis-(3-deoxy-3-(4-acetylbenzamido)-β-D-galactopyranosyl)sulfane; bis-(3-deoxy-3-[2-(3-carboxy)-naphthamido]-β-D-galactopyranosyl)sulfane; bis-[3-deoxy-3-(3,4-methylenedioxyl)benzamido]-β-D-galactopyranosyl)sulfane, bis-(3-deoxy-3-(4-methoxycarbonylbenzamido)-β-D-galactopyranosyl)sulfane; bis-(3-deoxy-3-(3-carboxybenzamido)-β-D-galactopyranosyl)sulfane; bis-(3-deoxy-3-(3-benzyloxy-5-hydroxy-benzamido)-β-D-galactopyranosyl)sulfane; bis-(3-deoxy-3-(3,5-dibenzyloxybenzamido)-β-D-galactopyranosyl)sulfane; bis-(3-deoxy-3-(3-benzyloxy-5-methoxy-benzamido)-β-D-galactopyranosyl)sulfane; bis-(3-deoxy-3-(3-benzyloxy-5-nonyloxy-benzamido)-β-D-galactopyranosyl)sulfane; bis-(3-deoxy-3-(3-hydroxy-5-methoxy-benzamido)-β-D-galactopyranosyl)-sulfane; bis-(3-deoxy-3-(3-hydroxy-5-nonyloxy-benzamido)-β-D-galactopyranosyl)sulfane, bis-(3-deoxy-3-[3-benzyloxy-5-(4-fluoro-benzyloxy)-benzamido]-β-D-galactopyranosyl)sulfane; bis-(3-deoxy-3-[3-methoxy-5-(4-methyl-benzyloxy)-benzamido]-β-D-galactopyranosyl)sulfane; or bis-(3-deoxy-3-(3-allyloxy-5-benzyloxy-benzamido)-β-D-galactopyranosyl)sulfane.

22. The composition according to claim 1, wherein the composition comprises a 3-triazolyl-galactoside, for example the composition comprises a general formula shown below: 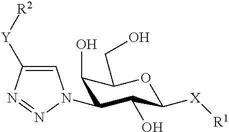
wherein the configuration of the pyranose ring is D-galacto;
X is selected from the group consisting of O, S, NH, CH₂, and NR⁴, or is a bond;
Y is selected from the group consisting of CH₂, CO, SO₂, SO, PO₂ and PO, phenyl, or is a bond;
R₁ is selected from the group consisting of: a saccharide; a substituted saccharide; D-galactose; substituted D-galactose; C3-[1,2,3]-triazol-1-yl-substituted D-galactose; hydrogen, an alkyl group, an alkenyl group, an aryl group, a heteroaryl group, and a heterocycle and derivatives thereof; and an amino group, a substituted amino group, an imino group, or a substituted imino group; and,
R² is selected from the group consisting of; hydrogen, an amino group, a substituted amino group, an alkyl group, a substituted alkyl group, an alkenyl group, a substituted alkenyl group, an alkynyl group, a substituted alkynyl group, an alkoxy group, a substituted alkoxy group, an alkylamino group, a substituted alkylamino group, an arylamino group, a substituted arylamino group, an aryloxy group, a substituted aryloxy group, an aryl group, a substituted aryl group, a heteroaryl group, a substituted heteroaryl group, and a heterocycle, a substituted heterocycle.

23. The composition according to claim 22, wherein the saccharide is selected from the group consisting of glucose, mannose, galactose, N-acetylglucosamine, N-acetylgalactosamine, fucose, fructose, xylose, sialic acid, glucuronic acid, iduronic acid, galacturonic acid, a disaccharide or an oligosaccharide comprising at least two of the above saccharides, and derivatives thereof; and wherein X is O or S; wherein Y is CO, SO₂, or a bond: wherein wherein R¹ is galactose, glucose or N-acetylglucosamine: wherein wherein R¹ is a substituted galactose, a substituted glucose, or a substituted N-acetylglucosamine; wherein the substituted galactose is a C3-[1,2,3]-triazol-1-yl-substituted galactose: wherein R² is an amine or an aryl group, or R² is a substituted phenyl group wherein the substituent is one or more selected from the group consisting of halogen, alkoxy, alkyl, nitro, sulfo, amino, hydroxy or carbonyl group.

24-30. (canceled)

31. The composition according to claim 22, wherein the composition is methyl 3-deoxy-3-(1H-[1,2,3]-triazol-1-yl)-1-thio-β-D-galactopyranoside; methyl 3-deoxy-3-(4-propyl-1H-[1,2,3]-triazol-1-yl)-1-thio-β-D-galactopyranoside; methyl 3-(4-methoxycarbonyl-1H-[1,2,3]-triazol-1-yl)-3-deoxy-1-thio-β-D-galactopyranoside; methyl 3-deoxy-3-(4-(1-hydroxy-1-cyclohexyl)-1H-[1,2,3]-triazol-1-yl)-1-thio-β-D-galactopyranoside; methyl 3-deoxy-3-(4-phenyl-1H-[1,2,3]-triazol-1-yl)-1-thio-β-D-galactopyranoside; methyl 3-deoxy-3-(4-p-tolylsulfonyl-1H-[1,2,3]-triazol-1-yl)-1-thio-β-galactopyranoside; methyl 3-(4-methylaminocarbonyl-1H-[1,2,3]-triazol-1-yl)-3-deoxy-1-thio-β-D-galactopyranoside; methyl 3-(4-butylaminocarbonyl-1H-[1,2,3]-triazol-1-yl)-3-deoxy-1-thio-β-D-galactopyranoside; methyl 3-(4-benzylaminocarbonyl-1H-[1,2,3]-triazol-1-yl)-3-deoxy-1-thio-β-D-galactopyranoside; methyl 3-{4-(3-hydroxyprop-1-ylaminocarbonyl)-1H-[1,2,3]-triazol-1-yl}-3-deoxy-1-thio-β-D-galactopyranoside; methyl 3-{4-[2-(N-morpholino)-ethylaminocarbonyl]-1H-[1,2,3]-triazol-1-yl}-3-deoxy-1-thio-β-D-galactopyranoside; methyl 3-(4-methylaminocarbonyl-1H-[1,2,3]-triazol-1-yl)-3-deoxy-β-D-galactopyranosyl-(1→4)-2-acetamido-2-deoxy-β-D-glucopyranoside, bis-(3-deoxy-3-(4-(methylaminocarbonyl)-1H-[1,2,3]-triazol-1-yl)-β-D-galactopyranosyl)sulfane, methyl 3-deoxy-3-{4-(2-fluorophenyl)-1H-[1,2,3]-triazol-1-yl}-1-thio-β-D-galactopyranoside; methyl 3-deoxy-3-{4-(2-methoxyphenyl)-1H-[1,2,3]-triazol-1-yl}-1-thio-β-D-galactopyranoside; methyl 3-deoxy-3-{4-(3-methoxyphenyl)-1H-[1,2,3]-triazol-1-yl}-1-thio-β-D-galactopyranoside; methyl 3 deoxy-3-{4-(4-methoxyphenyl)-H-[1,2,3]-triazol-1-yl}-1-thio-β-D-galactopyranoside; methyl 3-deoxy-3-{4-(3,5-dimethoxyphenyl)-1H-[1,2,3]-triazol-1-yl}-1-thio-β-D-galactopyranoside; methyl 3-deoxy-3-{4-(1-naphthyl)-1H-[1,2,3]-triazol-1-yl}-1-thio-β-D-galactopyranoside; methyl 3-deoxy-3-{4-(2-naphthyl)-1H-[1,2,3]-triazol-1-yl}-1-thio-β-D-galactopyranoside; methyl 3-deoxy-3-{4-(2-pyridyl)-1H-[1,2,3]-triazol-1-yl}-1-thio-β-D-galactopyranoside; methyl 3-deoxy-3-{4-(3-pyridyl)-1H-[1,2,3]-triazol-1-yl}-1-thio-β-D-galactopyranoside; methyl 3-deoxy-3-{4-(4-pyridyl)-1H-[1,2,3]-triazol-1-yl}-1-thio-β-D-galactopyranoside; O-{3-deoxy-3-[4-phenyl-[1H-[1,2,3]-triazol-1-yl]-β-D-galactopyranosy-l}-3-indol-carbaldoxim; O-{3-deoxy-3-[4-(methylaminocarbonyl)-1H-[1,2,3]-triazol-1-yl]-β-D-galactopyranosyl}-3-indol-carbaldoxim; O-{3-deoxy-3-[4-phenyl-[1H-[1,2,3]-triazol-1-yl]-β-D-galactopyranosy-1}-(2-hydroxy-5-nitro-phenyl)-carbaldoxim; O-{3-deoxy-3-[4-(methylaminocarbonyl)-1H-[1,2,3]-triazol-1-yl]-β-D-galactopyranosyl}-(2-hydroxy-5-nitro-phenyl)-carbaldoxim; O-{3-deoxy-3-[4-phenyl-[1H-[1,2,3]-triazol-1-yl]-β-D-galactopyranosy-1}-(2,5-dihydroxyphenyl)-carbaldoxim; O-{3-deoxy-3-[4-(methylaminocarbonyl)-1H-[1,2,3]-triazol-1-yl]-β-galactopyranosyl}-(2,5-dihydroxyphenyl)-carbaldoxim; O-{3-deoxy-3-[4-phenyl-[1H-[1,2,3]-triazol-1-yl]-β-galactopyranosy-l}-1-naphthyl-carbaldoxim; or O-{3-deoxy-3-[4-(methylaminocarbonyl)-1H-[1,2,3]-triazol-1-yl]-β-D-galactopyranosyl}-1-naphthyl-carbaldoxim.

32. The composition according to claim 1, wherein the composition comprises a general formula shown below: 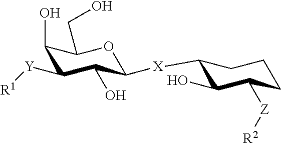
wherein the configuration of the pyranose ring is D-galacto;
X is selected from the group consisting of O, S, and SO;
Y and Z are independently selected from: CONH or a 1H-1,2,3-triazole ring; R¹ and R² are independently selected from the group consisting of: an alkyl group of at least 4 carbons, an alkenyl group of at least 4 carbons, an alkynyl group of at least 4 carbons; a carbamoyl group, a carbamoyl group substituted with an alkyl group, a carbamoyl group substituted with an alkenyl group, a carbamoyl group substituted with an alkynyl group, a carbamoyl group substituted with an aryl group, a carbamoyl group substituted with an substituted alkyl group, and a carbamoyl group substituted with an substituted aryl group; a phenyl group substituted with at least one carboxy group, a phenyl group substituted with at least one halogen, a phenyl group substituted with at least one alkyl group, a phenyl group substituted with at least one alkoxy group, a phenyl group substituted with at least one trifluoromethyl group; a phenyl group substituted with at least one trifluoromethoxy group, a phenyl group substituted with at least one sulfo group, a phenyl group substituted with at least one hydroxy group, a phenyl group substituted with at least one carbonyl group, and a phenyl group substituted with at least one substituted carbonyl group; a naphthyl group, a naphthyl group substituted with at least one carboxy group, a naphthyl group substituted with at least one halogen, a naphthyl group substituted with at least one alkyl group, a naphthyl group substituted with at least one alkoxy group, a naphthyl group substituted with at least one sulfo group, a naphthyl group substituted with at least one hydroxy group, a naphthyl group substituted with at least one carbonyl group, and a naphthyl group substituted with at least one substituted carbonyl group; a heteroaryl group, a heteroaryl group substituted with at least one carboxy group, a heteroaryl group substituted with at least one halogen, a heteroaryl group substituted with at least one alkoxy group, a heteroaryl group substituted with at least one sulfo group, a heteroaryl group substituted with at least one arylamino group, a heteroaryl group substituted with at least one hydroxy group, a heteroaryl group substituted with at least one halogen, a heteroaryl group substituted with at least one carbonyl group, and a heteroaryl group substituted with at least one substituted carbonyl group; and a thienyl group, a thienyl group substituted with at least one carboxy group, a thienyl group substituted with at least one halogen, a thienyl thienyl group substituted with at least one alkoxy group, a thienyl group substituted with at least one sulfo group, a thienyl group substituted with at least one arylamino group, a thienyl group substituted with at least one hydroxy group, a thienyl group substituted with at least one halogen, a thienyl group substituted with at least one carbonyl group, and a thienyl group substituted with at least one substituted carbonyl group.

33. The composition according to claim 32, further optionally characterized by at least one of the following:
X is O or S;
Y is CONH which is optionally linked via the N atom to the pyranose ring;
Z is CONH which is optionally linked via the N atom to the cyclohexane;
Y is a 1H-1,2,3-triazole ring which is optionally linked via the N1 atom to the pyranose ring;
R¹ is linked to the C4 atom of the 1H-1,2,3-triazole ring:
Z is a 1H-1,2,3-triazole ring which is optionally linked via the N1 atom to the cyclohexane;
R² is linked to the C4 atom of the 1H-1,2,3-triazole ring; and
R¹ and R² are independently selected from the group consisting of a carbamoyl group, an alkylated carbamoyl group, an alkenylated carbamoyl group, an arylated carbamoyl group, a phenyl group, a substituted phenyl group, a halogenated phenyl group, a fluorinated phenyl group, a chlorinated phenyl group, a brominated phenyl group, an alkylated phenyl group, an alkenylated phenyl group, a trifluoromethylated phenyl group, a methoxylated phenyl group, a trifluoromethoxylated phenyl group, a naphthyl group, a substituted naphthyl group, a heteroaryl group, a substituted heteroaryl group, a thienyl group, and a substituted thienyl group;
R¹ is an alkylated carbamoyl group, a fluorinated phenyl group, or a thienyl group;
R² is an alkylated carbamoyl group, a fluorinated phenyl group, or a thienyl group.

34-46. (canceled)

47. The composition according to claim 32, wherein the composition is selected from the group consisting of: ((1R,2R,3S)-2-hydroxy-3-(4-(N-(1-propyl)-carbamoyl)-1H-1,2,3-triazol-1-yl)cyclohexyl) 3-deoxy-(3-(4-(N-(1-propyl)-carbamoyl)-1H-1,2,3-triazol-1-yl))-β-D-galactopyranoside; ((1R,2R,3S)-2-hydroxy-3-(4-(2-fluorophenyl)-1H-1,2,3-triazol-1-yl)-cyclohexyl) 3-deoxy-3-(4-(2-fluorophenyl)-1H-1,2,3-triazol-1-yl)-1-thio-β-D-galactopyranoside; ((1R,2R,3S)-2-hydroxy-3-(4-(3-fluorophenyl)-1H-1,2,3-triazol-1-yl)-cyclohexyl) 3-deoxy-3-(4-(3-fluorophenyl)-1H-1,2,3-triazol-1-yl)-1-thio-β-D-galactopyranoside; ((1R,2R,3S)-2-hydroxy-3-(4-(4-fluorophenyl)-1H-1,2,3-triazol-1-yl)-cyclohexyl) 3-deoxy-3-(4-(4-fluorophenyl)-1H-1,2,3-triazol-1-yl)-1-thio-β-D-galactopyranoside; (1R,2R,3S)-2-hydroxy-3-(4-(3-thienyl)-1H-1,2,3-triazol-1-yl)-cyclohexyl) 3-deoxy-3-(4-(3-thienyl)-1H-1,2,3-triazol-1-yl)-1-thio-β-D-galactopyranoside; (1R,2R,3S)-2-hydroxy-3-(4-(N-(1-propyl)-carbamoyl)-1H-1,2,3-triazol-1-yl)-cyclohexyl) 3-deoxy-3-(4-(N-(1-propyl)-carbamoyl)-1H-1,2,3-triazol-1-yl)-1-thio-β-D-galactopyranoside, and (1R,2R,3S)-2-hydroxy-3-(4-chlorobenzamido)-cyclohexyl) 3-deoxy-3-(4-chlorobenzamido)-1-thio-β-D-galactopyranoside.

48. The composition according to claim 1, wherein the composition comprises a thiodigalactoside, for example the composition comprises a general formula (13) 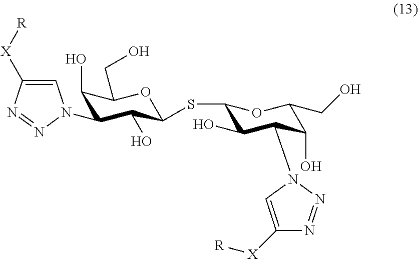
wherein the configuration of at least one of the pyranose rings is D-galacto; X is a bond; R is a phenyl group, which is substituted in any position with one or more substituents selected from the group consisting of methyl, ethyl, isopropyl, tert-butyl, fluoro, chloro, bromo, and trifluoromethyl or R is a thienyl group.

49-56. (canceled)

57. A method for treating or preventing an ocular angiogenesis in a subject, the method comprising administering a therapeutically effective amount of at least one composition to the subject, wherein the composition comprises an inhibitor of a galectin protein or a portion thereof, wherein the administering comprises contacting the subject with any of the pharmaceutical compositions according to claim 1.

58. A method for treating or preventing an ocular fibrosis in a subject, the method comprising administering a therapeutically effective amount of at least one composition to the subject, wherein the composition comprises an inhibitor of a galectin protein or a portion thereof, wherein the administering comprises contacting the subject with any of the pharmaceutical compositions according to claim 1.

59. The method according to claim 58, wherein administering comprises contacting the subject or tissue of the subject with a dose of at least: about 0.01 nanograms (ng) 1 to about 1 ng, about 1 ng to about 10 ng, about 10 ng to about 20 ng, about 20 ng to about 30 ng, about 30 ng to about 40 ng, about 40 ng to about 50 ng, about 50 ng to about 100 ng, 100 ng to about 200 ng, 200 ng to about 300 ng, about 300 ng to about 400 ng, about 400 ng to about 600 ng, about 600 ng to about 800 ng, about 1 microgram (μg) to about 5 μg, about 5 μg to about 20 μg, about 20 μg to about 40 μg, about 40 μg to about 60 μg, about 60 μg to about 80 μg, about 80 μg to about 100 μg, about 100 μg to about 200 μg, about 200 μg to about 300 μg, and about 300 μg to about 400 μg.

60-61. (canceled)

62. A kit for treating or preventing ocular angiogenesis in a subject or cells from the subject, the kit comprising:
a pharmaceutical composition that inhibits a galectin protein or portion, wherein the composition binds to the galectin protein and modulates a VEGF/VEGF receptor-2 pathway or modulates a TGF-β pathway or the composition modulates activity of the galectin protein;
instructions for use; and
a container,
wherein the pharmaceutical composition is described in claim 1.

63-72. (canceled)

73. The composition according to claim 1 comprises one or more of Bis-{3-0-[(5,6-difluoro-2/-/-1-benzopyran-2-on-3-yl)-methyl]-β-D-galactopyranosyl}sulfane, TD139, Compound 32, a compound having the following structure: 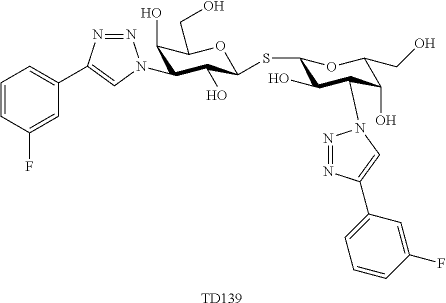
, or a portion or a homolog thereof wherein the composition inhibits angiogenesis.

74-135. (canceled)

136. The kit according to claim 62, wherein the pharmaceutical composition is further optionally characterized as follows:
the galectin protein is selected from the group of: galectin-1, galectin-3, galectin-7, and galectin-8;
the composition is selected from at least one of: a drug, a polymer, a protein, a peptide, a carbohydrate, a low molecular weight compound, an oligonucleotide, a polynucleotide, and a genetic material such as DNA or RNA, and the composition is formulated with a pharmaceutically suitable carrier or a diluent;
the kit further comprises an applicator or device for administering the composition, for example the applicator or the device is a syringe, a needle, a sprayer, a sponge, a gel, a strip, a tape, a bandage, a tray, a string, or a nanostructure; and
the kit further comprises a control for example Avastin™; (Bevacizumab™), Lucentis™ (Ranibizumab™), FOVISTA™ or Eylea™ (Aflibercept™), such that the control inhibits the ocular angiogenesis.

137. The method according to claim 58 further comprising prior to administering:
associating the ocular fibrosis in the subject with a disease or a condition for example selected from the group of: a surgery associated with scarring for example a glaucoma filtration surgery; neovascular glaucoma; a corneal injury; post-conjunctivitis scarring; pterygium, age related-macular degeneration (AMD) for example wet AMD or dry AMD; conversion from dry to wet AMD; proliferative diabetic retinopathy; diabetic macular adema; and corneal neovascularization (trachoma);
selecting the galectin protein from the group of galectin-1, galectin-3, galectin-7, and galectin-8;
selecting the composition from at least one of: a drug, a polymer, a protein, a peptide, a carbohydrate, a low molecular weight compound, an oligonucleotide, a polynucleotide, and a genetic material such as DNA or RNA;
engineering the composition and assaying the composition for binding to the galectin protein and modulating a VEGF/VEGF receptor-2 pathway, a TGF-β receptor pathway or the modulating expression of the galectin protein; and
observing after administering to the subject a reduction in a parameter or an indicator of the disease or the condition, for example the parameter or the indicator is a marker.
